(19) United States
(12) Patent Application Publication
(10) Pub. No.: US 2010/0203060 A1
(43) Pub. Date: Aug. 12, 2010

(54) INHIBITORS FOR GROWTH HORMONE AND RELATED HORMONES, AND METHODS OF USE THEREOF

(76) Inventor: Peter E. Lobie, Glendowie (NZ)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/600,335

(22) PCT Filed: May 30, 2008
(86) PCT No.: PCT/IB08/02585

§ 371 Date: Apr. 23, 2010

Related U.S. Application Data
(60) Provisional application No. 60/940,939, filed on May 30, 2007.

(51) Int. Cl. A61K 48/00; C07K 14/61; A61K 31/713; C07K 14/435; A61K 39/395; C07K 16/22; C07H 21/02; G01N 33/53; A61P 35/00

(52) U.S. Cl. 424/158.1; 514/44 R; 530/399; 514/44 A; 530/395; 530/389.2; 536/24.5; 435/7.1

(57) ABSTRACT

The present invention encompasses novel inhibitors for growth hormone and related hormones, including prolactin, and placental lactogen, and other hormones. The invention specifically encompasses antibodies, antibody fragments, and modifications thereof, as well as polynucleotides, such antisense polynucleotides, interfering RNAs, and small interfering RNAs, and uses thereof, for inhibition of one or more of such hormones. In particular aspects, the invention encompasses methods of producing such inhibitors, compositions comprising one or more of these inhibitors, and methods of inhibiting a cell, e.g., inhibiting cell proliferation, cell survival, or cell motility, especially for a cancer cell, using one or more of the inhibitors. The invention also encompasses methods of diagnosis and monitoring, and methods of treatment, especially for cancer, using one or more of the disclosed compositions or inhibitors.

### REFERENCE TO RELATED APPLICATIONS

This non-provisional application claims priority to U.S. Ser. No. 60/940,939, filed May 30, 2007, which is herein incorporated by reference in its entirety.

### FIELD OF THE INVENTION

The present invention relates to novel inhibitors for growth hormone and related hormones, particularly prolactin, and placental lactogen, and other hormones described herein. The invention relates specifically to antibodies, antibody fragments, and modifications thereof, as well as polynucleotides, such antisense polynucleotides, interfering RNAs, and small interfering RNAs, and uses thereof, for inhibition of growth hormone and/or related hormones. In particular aspects, the invention relates to methods of producing such inhibitors, compositions comprising one or more of these inhibitors, and methods of inhibiting a cell, e.g., inhibiting cell proliferation, cell survival, or cell motility, especially for a cancer cell, using one or more of the inhibitors. The invention also relates, in particular, to methods of diagnosis and monitoring, and methods of treatment, especially for cancer, using one or more of the disclosed compositions or inhibitors.

### BACKGROUND OF THE INVENTION

Human growth hormone (hGH) plays a central role in growth, development and sexual maturation and has been extensively studied since its isolation in 1944 (C. H. Li, H. M. Evans, Isolation of pituitary growth hormone, Science 99 (1944) 183-184). Endocrine hGH is secreted from the anterior pituitary, and exerts a direct effect on somatic growth regulation. This effect is mediated through interaction with the membrane bound growth hormone receptor (GHR) (reviewed in T. Zhu, E. L. Goh, R. Graichen, L. Ling, P. E. Lobie, Signal transduction via the growth hormone receptor, Cell Signal 13 (2001) 599-616; D. Le Roith, C. Bondy, S. Yakar, J. L. Liu, A. Butler, The somatomedin hypothesis: 2001, Endocr. Rev. 22 (2001) 53-74). As a primary mechanism, hGH promotes growth through stimulation of hepatic IGF-1 secretion (reviewed in D. Le Roith, C. Bondy, S. Yakar, J. L. Liu, A. Butler, The somatomedin hypothesis: 2001, Endocr. Rev. 22 (2001) 53-74). Yet, hGH also exhibits many IGF-1-independent effects on growth.

hGH secretion from the anterior pituitary is triggered by growth hormone releasing hormone (GHRH) and ghrelin, while it is negatively regulated by somatostatin (SS). However, synthesis of hGH also occurs at a number of extrapituitary sites within the body. Thus, hGH acts at the endocrine level, but also has important autocrine and paracrine activities (see, e.g., D. Le Roith, C. Bondy, S. Yakar, J. L. Liu, A. Butler, The somatomedin hypothesis: 2001, Endocr. Rev. 22 (2001) 53-74; N. Liu, H. C. Mertani, G. Norstedt, J. Tornell, P. E. Lobie, Mode of the autocrine/paracrine mechanism of growth hormone action, Exp. Cell. Res. 237 (1997) 196-206; S. Harvey, K. L. Hull, Growth hormone. A paracrine growth factor? Endocrine 7 (1997) 267-279). Extrapituitary secretion of hGH has been localised to numerous sites including discrete neuronal populations within the central nervous system, epithelial cells of the mammary gland, endothelial cells of blood vessel fibroblasts, thymic epithelial cells, and cells of the immune system including macrophages, B-cells, T-cells, and natural killer cells (see, e.g., N. Liu, H. C. Mertani, G. Norstedt, J. Tornell, P. E. Lobie, Mode of the autocrine/paracrine mechanism of growth hormone action, Exp. Cell. Res. 237 (1997) 196-206; S. Harvey, K. L. Hull, Growth hormone. A paracrine growth factor? Endocrine 7 (1997) 267-279, and references therein).

GH belongs to a family of hormones that includes prolactin (PRL) and placental lactogens (PL) along with lesser-known members, proliferin and proliferin-related protein, which can be locally produced by endothelial cells or neighbouring cells (reviewed by A M Corbacho et al., Journal of Endocrinology, 2002, 173, 219-238). The classical members of this family of peptide hormones, GH, PRL, and PL, are homologous proteins thought to have arisen from a common ancestral gene. PRL and GH are mainly secreted by the anterior pituitary of all vertebrates, while PL is present only in mammals and is secreted by the placenta. These three hormones share many structural and biological features. Similarity at the mRNA and protein levels between GH, PL, and PRL has been extensively characterised (Nicoll C S, Mayer G L & Russell S M 1986 Structural features of prolactins and growth hormones that can be related to their biological properties. Endocrine Reviews 7 169-203, Goffin V, Shiverick K T, Kelly P A & Martial J A 1996b Sequence-function relationships within the expanding family of prolactin, growth hormone, placental lactogen and related proteins in mammals. Endocrine Reviews 17 385-410; Kelly P A 1990 Growth hormone and prolactin. In Hormones from Molecules to Disease, pp 190-217. Eds E-E Baulieu & PA Kelly. Paris: Herman, Publishers in Arts and Science).

Targeting of specific pathways to stop cancer growth has emerged as an alternative strategy for new drug development because of less toxicity and high tolerability. For example, an increasing number of compounds directed against the EGFR and HER2 receptors have entered clinical development and are currently in clinical trials. As one particular example, Herceptin® has been developed as a humanised monoclonal antibody for targeting and blocking the function of HER2. In addition, Tykerb® has been developed as a dual tyrosine kinase inhibitor for epidermal growth factor receptor (EGFR) and human epidermal growth factor receptor 2 (HER-2). Still, there is a continuing need for new anti-cancer agents. In particular, there is a need to identify specific genes and proteins that can be targeted to inhibit cancer cell growth. The present invention meets this need and other needs.

### SUMMARY OF INVENTION

The invention encompasses inhibitory agents for the genes and the respective gene products of growth hormone (GH) and related hormones, such as one or more members of the human growth hormone gene cluster, a prolactin (PRL) gene, and/or a proliferin gene, and their respective gene (i.e., peptide) products. Members of the human growth hormone gene cluster include human growth hormone 1 (hGH1) gene, growth hormone 2 (hGH2) gene, human chorionic somatomammotropin hormone 1 (CSH1) gene; also known as human placental lactogen (PL, e.g., hPL-1, hPL-2, hPL-3)), human chorionic somatomammotropin hormone 2 (CSH2) gene, human chorionic somatomammotropin-like hormone (CSL) gene, human chorionic somatomammotropin-like 2 hormone (CSL-2) gene, humnan chorionic somatomammotropin-like 3 hormone (CSL-3) gene, human chorionic somatomammotropin-like 4 hormone (CSL-4) gene, or any variants thereof. Examples of genes encoding members of the human growth hormone gene cluster include but are not limited to the amino acid sequences described in GenBank Accession Nos. AAA72260, AAK69708, NP_{—}001308, NP_{—}002050, AAA98621, AAA39404, NP 851350, NP_{—}072171, NP_{—}066271, NP_{—}072170, NP_{—}001308, NP_{—}072167, and NP_{—}072166. Genes for prolactins include but are not limited to prolactin gene, prolactin-related protein gene, or any variants thereof. Examples of genes encoding a prolactin include but are not limited to the amino acid sequences described in GenBank Accession Nos. CAA38264, NP_{—}000939, CAA25214, CAA25108, AAH88370, CAA23829, and CAA38265. Genes for proliferins include but are not limited to proliferin gene, proliferin-related protein gene, or any variants thereof. Examples of genes encoding a proliferin include but are not limited to the amino acid sequence described in GenBank Accession No. 548671.

Gene products of the human growth hormone gene cluster include human growth hormone 1 (hGH1), growth hormone 2 (hGH2), human chorionic somatomammotropin hormone 1 (CSH1); also known as human placental lactogen (PL, e.g., hPL-1, hPL-2, hPL-3)), human chorionic somatomammotropin hormone 2 (CSH2), human chorionic somatomammotropin-like hormone (CSL), human chorionic somatomammotropin-like 2 hormone (CSL-2), human chorionic somatomammotropin-like 3 hormone (CSL-3), human chorionic somatomammotropin-like 4 hormone (CSL-4), or any variants thereof. Gene products of prolactin genes include but are not limited to prolactin and prolactin-related protein. Gene products for proliferin include but are not limited to proliferin and proliferin-related protein.

The inhibitory agents for growth hormone in accordance with the invention embrace inhibitors such as antibodies, and polynucleotides, including antisense polynucleotides, interfering RNAs (iRNAs), and small interfering RNAs (siRNAs).

In one aspect, the present invention encompasses antibodies directed to GH and/or a related hormone, or variants thereof, and antibody fragments, and modifications thereof. The antibodies of the invention are characterized by their ability to specifically bind to an hGH, prolactin, or a prolifern polypeptide, and/or variant a thereof, such as for example, hGH1, hGH2, hPRL or hPL. The hGH, hPRL, and/or hPL polypeptides to which the antibodies of the invention specifically bind include one or more conformational and/or sequential epitopes on said hGH, hPRL, and/or hPL polypeptide. Conformational and sequential epitopes on an hGH polypeptide, or variant thereof, include but are not limited to the conformational epitopes shown in Tables 2, and the sequential epitopes shown in Table 3, respectively, as described herein. Conformational epitopes on an hPRL polypeptide include but are not limited to the conformational epitopes shown in Table 5, as described herein. The antibodies of the invention may comprise an antigenic determinant of a growth hormone polypeptide, such as hGH, hPRL and/or hPL, such as an antigenic determinant described herein in Tables 1 and 4.

In a particular aspect, the invention encompasses a polyclonal or monoclonal antibody directed to GH, and/or a related hormone, particularly hGH and/or variants thereof, hPRL, or hPL, and antibody fragments, and modifications thereof. In certain aspects, the antibodies are directed to a polypeptide, e.g., at least one of SEQ ID NOs: 1-26, preferably one of SEQ ID Nos: 10-26, or a modified sequence thereof. In various aspects, the antibodies are directed to the native polypeptide, any peptides derived from this polypeptide, any modifications of these polypeptides or peptides (e.g., where primary structure is based on the sequence of the hormone), or any polypeptides or peptides which mimic the 3-D conformation of the hormone.

In another particular aspect, the invention encompasses a polyclonal antibody, antibody fragment, or modification thereof.

In a further particular aspect, the invention encompasses a monoclonal antibody, antibody fragment, or modification thereof.

In another aspect, the present invention encompasses inhibitory polynucleotides for GH and/or a related hormone, particularly hGH and/or variants thereof, hPRL, or hPL, including, for example, antisense polynucleotides, iRNAs, and siRNAs.

In particular aspects, the inhibitory agent is a polynucleotide adapted to inhibit GH and/or a related hormone in use. In specific aspects, the polynucleotide comprises at least one of the nucleotide sequences of SEQ ID NO: 27-96, or a modified sequence thereof. More particularly, the agent is selected from the group consisting of: an antisense nucleic acid directed to a GH and/or a related hormone transcript; a nucleic acid adapted to express such antisense in use; iRNA directed to a GH and/or a related hormone transcript; and a nucleic acid adapted to express such iRNA in use.

In other particular aspects, the present invention encompasses an isolated iRNA (e.g., siRNA) directed to the GH and/or a related hormone transcript, or a nucleic acid adapted in use to express an iRNA directed to the GH and/or a related hormone transcript. In specific aspects, an isolated iRNA comprises any one of the nucleotide sequences of SEQ ID NOs: 33-60 or 97-98, or a modified sequence thereof. In some aspects, the isolated iRNA may comprise a sense strand and an antisense strand which form a duplex. Such antisense polynucleotides and iRNAs, in particular, siRNAs, can inhibit expression of a polynucleotide and/or peptide product for GH and/or a related hormone, e.g., SEQ ID NO: 27-32, or modified sequences thereof.

In other aspects, the invention encompasses expression vectors, as well as host cells, for producing these antisense polynucleotides or iRNAs. The invention also features a host cell, for example, a microbial host cell, comprising at least one expression vector.

In another aspect, the invention encompasses a method of inhibiting genes and the respective gene (i.e. peptide) products of GH and/or related hormones, particularly hGH and/or variants thereof, hPRL, or hPL, the method comprising: contacting GH and/or the related hormone with an antibody, or antibody fragment, or modification thereof in accordance with the invention. In particular aspects, the method is used to decrease activity or expression levels of GH and/or a related hormone.

In a further aspect, the invention encompasses a method of inhibiting genes and the respective gene (i.e. peptide) products of GH and/or a related hormone, particularly hGH and/or variants thereof, hPRL, or hPL, the method comprising: contacting a polynucleotide for GH and/or a related hormone with a polynucleotide inhibitor, such as antisense, iRNA, or siRNA, in accordance with the invention. In particular aspects, the method is used to decrease activity or expression levels of GH and/or a related hormone.

In a still further aspect, the invention encompasses a method of inhibiting the interaction of GH and/or a related hormone, particularly hGH and/or variants thereof, hPRL, or hPL, the method comprising: contacting said receptor with an antibody, or antibody fragment, or modification thereof in accordance with the invention.

In an additional aspect, the invention encompasses a method of inhibiting proliferation, survival and/or motility of a cell, in particular a tumor cell (e.g., an epithelial tumor cell such as one derived from breast cancer, colon cancer, lung cancer, prostate cancer, endometrial cancer, and/or endometriosis). Specifically, the method can comprise use of at least one inhibitory agent of GH and/or a related hormone, particularly hGH and/or variants thereof, hPRL, or hPL, as described herein, comprising contacting the cell with the inhibitor. In particular aspects, the inhibitory agent is an antibody, or antibody fragment, or modification thereof, as describe herein. In other particular aspects, the inhibitory agent is a polynucleotide inhibitor, or fragment, or modification thereof, such as such as antisense, iRNA, or siRNA, as describe herein.

In a related aspect, the present invention encompasses a method of treating or preventing a disorder such as cancer (including but not limited to breast cancer, colon cancer, lung cancer, prostate cancer, endometrial cancer), a cell proliferation disorder (including but not limited to endometriosis) and/or a cell survival disorder, in a subject in need thereof., the method comprising: contacting said cell with an inhibitory agent of human growth hormone and/or related hormone, or variants thereof. The hGH inhibitory agent may be administered alone or in combination with a second therapeutic compound (simultaneously or in succession), such as a chemotherapeutic or anti-neoplastic agent. For example, contact of said cell with said inhibitory agent may be achieved by administering to said subject an effective amount of one or more of the inhibitory agents described herein, including but not limited to the antibodies, antibody fragments, or modifications thereof as described herein.

In another related aspect, the present invention encompasses a method of treating or preventing a disorder such as cancer (including but not limited to breast cancer, colon cancer, lung cancer, prostate cancer, endometrial cancer), a cell proliferation disorder (including but not limited to endometriosis) and/or a cell survival disorder, in a subject in need thereof., the method comprising: contacting said cell with an inhibitory agent of hGH and/or related hormone, or variants thereof. The hGH inhibitory agent may be administered alone or in combination with a second therapeutic compound (simultaneously or in succession), such as a chemotherapeutic or anti-neoplastic agent. For example, contact of said cell with said inhibitory agent may be achieved by administering to said subject an effective amount of one or more of the inhibitory agents described herein, including but not limited to the polynucleotide inhibitors, such as antisense, iRNA, or siRNA, in accordance with the invention.

In another aspect, the invention encompasses a method of detecting the presence or levels of GH and/or a related hormone, particularly hGH and/or variants thereof, hPRL, or hPL, in a sample, the method comprising: contacting said sample with one or more of the antibodies, antibody fragments, or modifications thereof, as herein described. In particular aspects, the sample is a biological fluid or tissue.

In yet another aspect, the invention encompasses a method of detecting the presence or levels of GH and/or a related hormone, particularly hGH and/or variants thereof, hPRL, or hPL, in a sample, the method comprising: contacting said sample with one or more of the polynucleotides, fragments, or modifications thereof, as herein described. In particular aspects, the sample is a biological fluid or tissue.

In an additional aspect, the invention encompasses a method of diagnosing or monitoring a disorder in a subject (e.g., a human subject), such as cancer (including but not limited to an epithelial cancer such as breast cancer, colon cancer, lung cancer, prostate cancer, and/or endometrial cancer), a cell proliferation disorder (including but not limited to endometriosis), and/or a cell survival disorder, the method comprising: contacting a sample from said subject with an inhibitory agent for the genes and respective peptide products of growth hormone (GH) and related hormones, particularly human growth hormone (hGH) and variants thereof, as described herein, and determining the level of antibody binding in said sample compared to the level of binding in a control sample. An increase in the level of antibody binding in said sample as compared to a control sample indicates the presence of a cancer, cell proliferation disorder and/or cell survival disorder. In particular aspects, the sample from the subject is a biological fluid or tissue.

In a particular aspect, the invention encompasses a method of diagnosing or monitoring a disorder in a subject (e.g., a human subject), such as cancer (including but not limited to an epithelial cancer such as breast cancer, colon cancer, lung cancer, prostate cancer, and/or endometrial cancer), a cell proliferation disorder (including but not limited to endometriosis), and/or a cell survival disorder, the method comprising: contacting at least one antibody as described herein with a sample from the subject; and, determining the level of antibody binding in said sample compared to the level of binding in a control sample, whereby an increase in the level of antibody binding in said sample as compared to a control sample indicates the presence of a cancer, cell proliferation disorder and/or cell survival disorder.

In another particular aspect, the invention encompasses a method of diagnosing or monitoring a disorder in a subject (e.g., a human subject), such as cancer (including but not limited to an epithelial cancer such as breast cancer, colon cancer, lung cancer, prostate cancer, and/or endometrial cancer), a cell proliferation disorder (including but not limited to endometriosis), and/or a cell survival disorder, the method comprising: contacting at least one antibody as described herein with a sample from the subject; and, determining the level of antibody binding in said sample compared to the level of binding in a control sample, whereby an increase in the level of antibody binding in said sample as compared to a control sample indicates the presence of a cancer, cell proliferation disorder and/or cell survival disorder.

In a specific aspect, the method of diagnosis or monitoring comprises comparing the level of GH and/or a related hormone, particularly hGH and/or variants thereof, hPRL, or hPL, in said test sample with a standard or base level. In particular aspects, this method of the invention employs ELISA. Alternatively, or additionally, the method employs one or more of RIA, immunoprecipitation, Western blotting, immunohistochemical staining, affinity chromatography, competitive binding assays, and agglutination assays.

In another aspect, the invention encompasses a composition, for example, a pharmaceutical composition, comprising at least one of the antibodies, or antibody fragments, or modifications thereof, as herein described. In specific aspects, the composition can include a combination with one or more pharmaceutically acceptable diluents, carriers, and/or excipients.

In still another aspect, the invention encompasses a composition, for example, a pharmaceutical composition, comprising at least one of the polynucleotides, or fragments, or modifications thereof, as herein described. In specific aspects, the composition can include a combination with one or more pharmaceutically acceptable diluents, carriers, and/or excipients.

In a further aspect, the invention encompasses the use of one or more of the antibodies, or antibody fragments, or modifications thereof, as herein described, in the manufacture of a medicament for the treatment of a disorder, especially cancer, and particularly breast cancer, colon cancer, lung cancer, prostate cancer, endometrial cancer, or endometriosis in a subject.

In a still further aspect, the invention encompasses the use of one or more of the polynucleotides, or fragments, or modifications thereof, as herein described, in the manufacture of a medicament for the treatment of a disorder, especially cancer, and particularly breast cancer, colon cancer, lung cancer, prostate cancer, endometrial cancer, or endometriosis, in a subject.

In another aspect, the invention encompasses the use of an antibody, or antibody fragment, or modification thereof, as herein described, in a method for the isolation or purification of GH and/or a related hormone, particularly hGH and/or variants thereof, hPRL, or hPL.

In yet another aspect, the invention encompasses the use of a polynucleotide, or fragment, or modification thereof, as herein described, in a method for the identification of a polynucleotide (e.g., gene or transcript) GH and/or a related hormone, particularly hGH and/or variants thereof, hPRL, or hPL.

In a further aspect, the invention encompasses a kit for diagnosing, monitoring, or treating a disorder especially cancer, and particularly breast cancer, colon cancer, lung cancer, prostate cancer, endometrial cancer, or endometriosis, in a subject, the kit comprising at least one of the antibodies, or antibody fragments, or modifications thereof, as herein described.

In still a further aspect, the invention encompasses a kit for diagnosing, monitoring, or treating a disorder especially cancer, and particularly breast cancer, colon cancer, lung cancer, prostate cancer, endometrial cancer, or endometriosis, in a subject, the kit comprising at least one of the polynucleotides, or fragments, or modifications thereof, as herein described.

In particular aspects, the kits can comprise: a) at least one inhibitory agent (e.g., polynucleotide or antibody) for GH and/or a related hormone, particularly hGH and/or variants thereof, hPRL, or hPL as set out herein; and b) optionally, instructions for use.

In other particular aspects, a kit according to the invention may further comprise one or more control samples comprising a known level of GH and/or a related hormone, particularly hGH or variants thereof, hPRL, or hPL, or a fragment, or modification thereof. In another aspect, the kit may further comprise soluble hormone isolated from a human subject.

In other particular aspects, a kit comprises a composition of the invention for monitoring, diagnosis, or treatment, especially for cancer, and particularly breast cancer, colon cancer, lung cancer, prostate cancer, endometrial cancer, or endometriosis, in accordance with the disclosed methods.

In a related aspect, the invention also provides the use of GH and/or a related hormone, particularly hGH or variants thereof, hPRL, or hPL, or a fragment, or modification thereof, in the manufacture of a kit for diagnosing, monitoring, or treating a disorder, especially cancer, and particularly breast cancer, colon cancer, lung cancer, prostate cancer, endometrial cancer, or endometriosis.

In various aspects, the methods of the invention utilize in vivo or in vitro expression systems. In other aspects, the methods employ polynucleotides or antibodies produced by recombinant, synthetic, or semi-synthetic means, or polynucleotides or antibodies produced by endogenous means (e.g., naturally occurring components).

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

Other aspects and embodiments of the invention are described herein below.

### BRIEF DESCRIPTION OF THE DRAWINGS

These and other aspects of the present invention, which should be considered in all its aspects, will become apparent from the following description, which is given by way of example only, with reference to the accompanying figures:

FIGS. 1A-1D: Demonstration of forced expression of autocrine-hGH in RL95-2 endometrial carcinoma cell line and its effect on cell viability. RL95-2 cells were stably transfected with hGH cDNA using pcDNA3 vector as vehicle. (FIG. 1A) The Level of hGH mRNA and hGH receptor was determined by RT-PCR. (FIG. 1B) Measurement of hGH secretion to media by vector and hGH cDNA transfected RL95-2 cells by ELISA. Effect of forced expression of hGH on RL95-2 cells viability was evaluated by MIT assay, FIG. 1C represents 10% FBS medium and FIG. 1D represents serum-deficit medium (0.2%) conditions. *p-value<0.001, **p-value<0.05.

FIGS. 2A-2D: Autocrine-hGH production by RL95-2 human endometrial carcinoma cells enhances cell proliferation and cell survival. (FIG. 2A) Growth curve of RL95-2-Vector and RL95-2-hGH cells in 10% FBS medium. (FIG. 2B) Effect of autocrine-hGH production on cell cycle progression indicated by nuclear incorporation of BrdU and (FIG. 2C) effect on apoptosis by TUNEL assay, apoptosis induced by serum withdrawal for 48 hours. (FIG. 2D) Effect of autocrine-hGH expression on expression of various cell cycle regulator, anti-apoptotic, pro-apoptotic, and oncogene markers by real-time PCR. *p-value<0.001, ** value<0.05.

FIGS. 3A-3D: Autocrine-hGH production by human endometrial carcinoma cells results in enhanced anchorage-independent growth, foci formation, filling of luminal space, and disturbance of acinar morphology. (FIG. 3A) Growth curve of RL95-2-Vector and RL95-2-hGH-stable cells in suspension culture, visualization of suspension culture colonies in 10% FBS medium. (FIG. 3B) Soft agar colony formation of RL95-2-Vector and RL95-2-hGH-stable cells in 10% FBS medium, visualization of colonies under 150× magnification. (FIG. 3C) Matrigel colonies of RL95-2-Vector and RL95-2-hGH-stable cells, graph represent total numbers of colonies with disturbed acinar morphology. Images of colonies were captured under 400× magnification. (FIG. 3D) Foci formation of RL95-2-Vector and RL95-2-hGH-stable cells in 10% FBS medium. *p-value<0.001, **p-value<0.05.

FIGS. 4A-4D: Autocrine-hGH production by human endometrial carcinoma cells stimulates a mesenchymal phenotype and results in increased cell motility and acquisition of an invasive phenotype. (FIG. 4A) The morphology of RL95-2-Vector and RL95-2-hGH-stable cells cultured in 10% FBS medium on plastic was examined by bright-field microscopy under 400× magnification. (FIG. 4B) Wound-healing assay, wounded areas were examined under 100× magnification. The motility (FIG. 4C) and invasion (FIG. 4D) of RL95-2-Vector and RL95-2-hGH-stable cells were determined by Transwell chamber assay. *p-value<0.001, **p-value<0.05.

FIGS. 5A-5C: Demonstration of forced expression of autocrine-hGH in AN3 cell line and its functional characteristics. (FIG. 5A) The level of hGH mRNA was determined by RT-PCR. (FIG. 5B) Growth curve AN3-Vector and AN3-hGH-stable cells in 10% FBS medium. (FIG. 5C) Soft agar colony formation of AN3-Vector and AN3-hGH-stable cells in 10% FBS medium, visualization of colonies under 150× magnification. *p-value<0.001, **p-value<0.05.

FIGS. 6A-6D: Effect of rabbit hGH-antisera on RL95-2 cells. Effect on RL95-2 cell viability by treatment with hGH-antisera was evaluated by MIT assay. (FIG. 6A) 10% FBS medium; and (FIG. 6B) serum-deficit (0.2%) medium; NRC=normal rabbit serum; Cells were treated twice with antibodies at a 48 hr interval and the assay terminated at 4 days. (FIG. 6C) Expression pattern of cell cycle and apoptosis gene markers after treatment with hGH-antisera relative to non-specific rabbit IgG. (FIG. 6D) Effect of increasing concentrations of antibodies to hGH on 3/7 caspase activity assessed by CASPASE-GLO™ 3/7 ASSAY kit (Promega) relative to normal rabbit serum.

FIG. 7: hGH gene expression pattern in various cell lines. Expression profile of hGH gene in various cell lines were evaluated by real-time PCR. Bars represent relative expression of hGH gene against MCF7 (ATCC) cell line. Key: r2=0.984; SF=serum−free; HP=High passage number; vec=vector.

FIGS. 8A-8D: Sequence information for GH and related hormones. (FIG. 8A) CLUSTALW amino acid sequence alignment for hPRL, GH1, GH2, CSH1, CSH2, hCSL, hCSL-2, hCSL-3, and hCSL-4. Signal peptide sequences are shown with underlining. (FIG. 8B) Amino acid sequences for hPRL, GH1, GH2, CSH1, CSH2, hCSL, hCSL-2, hCSL-3, and hCSL-4. Signal peptide sequences are shown with underlining. (FIG. 8C) CLUSTALW nucleotide sequence alignment for GH1, GH2, CSHL, CSH2, CSH1, and hPRL. (FIG. 8D) Nucleotide sequences for GH1, GH2, CSHL, CSH2, CSH1, and hPRL.

FIGS. 9A-9C: Depletion of hGH mRNA by siRNA in endometrial carcinoma cells enhances apoptosis: Depletion of hGH mRNA by two siRNA constructs (sihGH5 and sihGH6 (SEQ ID NO: 97 and SEQ ID NO: 98)) increased apopototic activity in RL95-2 cells (apoptosis assessed by caspase 3/7 activity) (FIGS. 9A and 9B); Real-time PCR quantification analysis demonstrated a depletion of hGH gene expression in RL95-2 cells using sihGH5 and sihGH6 (FIG. 9C).

### DETAILED DESCRIPTION

The following is a description of the present invention, including preferred embodiments thereof, given in general terms. The invention is further elucidated from the disclosure given under the section “Examples” which provides experimental data supporting the invention and specific examples thereof.

### DEFINITIONS

The term “antibody” (e.g., “GH antibody” or like term) should be understood in the broadest possible sense and is intended to include intact monoclonal antibodies and polyclonal antibodies. It is also intended to cover fragments and other modifications so long as they exhibit the desired biological activity. Antibodies encompass immunoglobulin molecules and immunologically active portions of immunoglobulin (Ig) molecules, i.e., molecules that contain an antigen binding site that specifically binds (immunoreacts with) an antigen. These include, but are not limited to, polyclonal, monoclonal, chimeric, single chain, Fc, Fab, Fab′, and Fab₂ fragments, and a Fab expression library. Antibody molecules relate to any of the classes IgG, IgM, IgA, IgE, and IgD, which differ from one another by the nature of heavy chain present in the molecule. These include subclasses as well, such as IgG₁, IgG₂, and others. The light chain may be a kappa chain or a lambda chain. Reference herein to antibodies includes a reference to all classes, subclasses, and types. Also included are chimeric antibodies, for example, monoclonal antibodies or fragments thereof that are specific to more than one source, e.g., a mouse or human sequence. Further included are camelid antibodies or nanobodies. It will be understood that each reference to “antibodies” or any like term, herein includes intact antibodies, as well as any fragments, and any modifications thereof.

The term “antigen binding site” or “antigen binding portion” refers to the part of the immunoglobulin molecule, or fragment, or modification thereof, that participates in antigen interaction. The antigen binding site is generally formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions”. Thus, the term “framework region” or “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen binding surface. The antigen binding surface is generally complementary to the three-dimensional surface of a bound antigen, and the three hypervariable regions of each of the heavy and light chains are referred to as “complementarity-determining regions,” or “CDRs.” The assignment of amino acids to each domain is in accordance with accepted definitions (see, e.g., Kabat Sequences of Proteins of Immunological Interest, National Institutes of Health, Bethesda, Md., 1987 and 1991; and Chothia &Lesk J. Mol. Biol. 196:901-917, 1987, Chothia et al. Nature 342:878-883, 1989).

“Altered” polynucleotides, as used herein, include those with deletions, insertions, or substitutions of different nucleotides resulting in polynucleotides that encode the same or functionally equivalent. The encoded polypeptides and antibodies may also be “altered” and contain deletions, insertions, or substitutions of amino acid residues which produce a silent change and result in functionally equivalent sequences. Deliberate amino acid substitutions may be made on the basis of similarity in polarity, charge, solubility, hydrophobicity, hydrophilicity, and/or the amphipathic nature of the residues as long as at least one biological activity (e.g., stimulation of cell proliferation, cell survival, or cell motility) or immunogenic or immunological activity is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine. Guidance in making substitutions and/or deletions or additions can be obtained, for example, by alignments to related sequences, as shown in the figures, herein.

The terms “cancer” and “cancerous” refer to a physiological condition in mammals that is typically characterized by abnormal or unregulated cell proliferation, cell survival, and/or cell motility. Cancer and cancer pathology can be associated, for example, with metastasis, interference with the normal functioning of neighbouring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc. Specifically included are breast cancers, which can include epithelial tumours, nonepithelial tumours, carcinomas, for example, carcinomas in situ, as well as invasive breast cancers. Also included are colon cancers, lung cancers, prostate cancers, endometrial cancers, and endometriosis, among other conditions.

The terms “complementary” or “complementarity,” as used herein, refer to the natural binding of polynucleotides under permissive salt and temperature conditions by base-pairing. For the sequence A-G-T, the complementary sequence is T-C-A, the reverse complement is A-C-T, and the reverse sequence is T-G-A. Complementarity between two single stranded molecules may be partial, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands and in the design and use of iRNAs and PNAs.

The term “derivative,” as used herein, refers to the chemical modification of a polynucleotide, or a polynucleotide complementary thereto. Such modifications include, for example, replacement of hydrogen by an alkyl, acyl, or amino group. In preferred aspects, a polynucleotide derivative encodes a polypeptide or antibody which retains the biological or immunological function of the natural molecule. A derivative polypeptide or antibody is one which is modified by glycosylation, pegylation, or any similar process which retains one or more biological functions (e.g., effect on cell proliferation, cell survival, or cell motility) or immunogenic function of the sequence from which it was derived. In reference to antibodies, the term “derivatives” includes, for example, hybrid and recombinant antibodies. Hybrid and recombinant versions of an antibody include, for example, humanised antibodies, diabodies, triabodies, and single chain antibodies.

As used herein, the term “epitope” includes any polypeptide or peptide determinant capable of specific binding to an immunoglobulin, or a related molecule, e.g., an scFv, or a T cell receptor. Epitopic determinants generally include chemically active surface groupings of molecules such as amino acids and/or sugar side chains, and usually have specific three dimensional structural characteristics, as well as specific charge characteristics. Epitopes include two main types, namely, sequential epitopes (SEs), where the antibody binds to a contiguous stretch of amino acid residues that are linked by peptide bond, and conformational epitopes (CEs), where the antibody binds to non-contiguous residues, brought together by folding of polypeptide chain. Conformational epitopes are also referred to herein as native epitopes. It is known from the analyses of the crystal structures of antigen-antibody complexes that, to be recognized by an antibody, the residues must be generally accessible for interactions, and thus presented near the surface of antigen. A commercially available algorithm was used to predict SE and CE An antibody is said to specifically bind an antigen when the dissociation constant is <1 μM; preferably <100 nM and most preferably <10 nM.

The term “expression” includes production of polynucleotides and polypeptides, in particular, the production of RNA (e.g., mRNA) from a gene or portion of a gene, and includes the production of a an amino acid sequence encoded by an RNA or gene or portion of a gene, and the appearance of a detectable material associated with expression. For example, the formation of a complex, from a polypeptide-polypeptide interaction, polypeptide-nucleotide interaction, or the like, is included within the scope of the term “expression”. Another example is the binding of a binding ligand, such as a hybridization probe or antibody, to a gene or other polynucleotide or oligonucleotide, a polypeptide or a protein fragment, and the visualization of the binding ligand. Thus, increased intensity of a spot on a microarray, on a hybridization blot such as a Northern blot, or on an immunoblot such as a Western blot, or on a bead array, or by PCR analysis, is included within the term “expression”.

“Growth hormone and related hormones” as used herein refers to hormones with similar sequences and/or functions, including, but not limited to growth hormone (GH); prolactin (PRL) and prolactin-related protein; the human growth hormone cluster of genes, including growth hormone 1 (GH1), growth hormone 2 (GH2), chorionic somatomammotropin hormone 1 (CSH1; also called placental lactogen (PL, PL-1, PL-2, and PL-3)), chorionic somatomammotropin hormone 2 (CSH2), chorionic somatomammotropin-like hormone (CSL), chorionic somatomammotropin-like 2 hormone (CSL-2), chorionic somatomammotropin-like 3 hormone (CSL-3), andchorionic somatomammotropin-like 4 hormone (CSL-4); and also proliferin and proliferin-related protein. Human hormones are specifically included in this definition. The hormones of the invention include multiple sequence designators, for example, GH1 is also designated as GH, GHN, and GH-N (growth hormone normal); GH2 is also designated GHL, GHV and GH-V (growth hormone variant); CSL is also designated as CSHL1, CS-5, CSHP1, and hCS-L; CSH1 is also designated as PL, hCS-A, and CSMT; GH2 is also designated as GHL, GHV, GH-V (growth hormone variant); and CSH2 is also designated as CSB, CS-2, and hCS-B. As used herein, the terms chorionic somatomammotropin and placental lactogen, and their respective sequence designators, may be used interchangeably. It will be understood that each reference to a hormone (e.g., hGH, hPRL, or hPL), or like term, herein, will include the full length sequences as well as any fragments, or modifications (including variants) thereof.

An “inhibitory agent”, and its “inhibition” or “inhibiting” of GH and/or a related hormone, particularly hGH and/or variants thereof, hPRL, or hPL, is intended to refer to aspects of blocking or reducing biological activity and/or expression levels of the genes and respective gene products of GH and/or related hormones. While it may be desirable to completely inhibit the activity of the hormone, this need not be essential. “Inhibition” of may occur at the level of expression and production of the hormone (e.g., transcriptional or translational levels) or by targeting hormone function, for example. The terms “inhibit” or “inhibition” of GH and/or a related hormone, particularly hGH and/or variants thereof, hPRL, or hPL, as used herein, refer to a decrease, for example, in DNA levels (e.g., decreased DNA synthesis, increased turnover, and/or decreased stability), RNA levels (e.g., decreased transcription, increased turnover, and/or decreased stability), or polypeptide levels (e.g., decreased translation, increased turnover, and/or decreased stability) or activity, or post-translational modification. An inhibitory agent can also decrease or block the activities or expression levels of downstream or upstream agents in the hormone pathway. In certain aspects, an inhibitory agent can specifically bind with or react with a hormone as described herein. For an antibody, “specifically bind” or “specifically immunoreacts with” is meant that the antibody reacts with one or more antigenic determinants of the desired antigen and does not react (i.e., bind) with other polypeptides or binds at much lower affinity (e.g., K_{d}>10⁻⁶) with other polypeptides.

In particular aspects, the inhibitory agents of the invention are useful for inhibiting cell proliferation, cell survival, and/or cell motility, especially for cancer cells, as described herein. In particular, the agents may be useful for one or more of: decreasing cell proliferation (e.g., by decreasing cell division), increasing cell death (e.g., by increasing apoptosis or necrosis), or decreasing cellular invasion and/or metastasis (e.g., by decreasing cytoskeletal activity, cell motors). While the invention is generally directed to inhibitors of GH and/or a related hormone, particularly hGH and/or variants thereof, hPRL, or hPL, in some circumstances, there may be beneficial aspects in maintaining or increasing hormone levels. The disclosed polynucleotides and polypeptides can also be used for such purposes, in accordance with well-known methods.

The term “modified” refers to sequence alterations and to sequence fragments, variants, and derivatives, as described herein. The term includes polypeptides, polynucleotides, antibodies, and like agents described herein.

The term “monoclonal antibody,” “MAb,” or “monoclonal antibody composition,” as used herein, refers to a population of antibody molecules that contain a molecular species of antibody molecule including a unique light chain gene product and a unique heavy chain gene product. In particular, the complementarity determining regions (CDRs) of the monoclonal antibody are unique. MAbs include an antigen binding site capable of immunoreacting with a particular epitope of the antigen characterized by a unique binding affinity for it.

The term “oligonucleotide” refers to a polynucleotide, typically a probe or primer, including, without limitation, single stranded DNAs, single or double stranded RNAs, RNA:DNA hybrids, and double stranded DNAs. Oligonucleotides, such as single stranded DNA probe oligonucleotides, are often synthesized by chemical methods, for example using automated oligonucleotide synthesizers that are commercially available, or by a variety of other methods, including in vitro expression systems, recombinant techniques, and expression in cells and organisms.

By “purified antibody” is meant antibody which is at least 60%, by weight, free from proteins and naturally occurring organic molecules with which it is naturally associated. Preferably, the preparation is at least 75%, more preferably 90%, and most preferably at least 99%, by weight, antibody, e.g., a GPR30 specific antibody. A purified antibody may be obtained, for example, by affinity chromatography using recombinantly-produced protein or conserved motif peptides and standard techniques.

By “purified” or “isolated” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

An isolated nucleic acid is one that is free of the genes or sequences which, in the naturally occurring genome of the organism from which the given nucleic acid is derived, flank the sequence. For example, “isolated DNA or RNA” encompasses, for example, cDNA, cloned genomic DNA, synthetic DNA, and siRNA constructs.

By “an effective amount” is meant an amount of a composition, alone or in a combination, required to produce a clinically desirable result. For example, for a cancer therapeutic, an effective amount of the inhibitors described herein reduce or prevent the growth or invasiveness of a tumors in a mammal. The effective amount of active compound(s) varies depending upon the route of administration, age, body weight, and general health of the subject. Ultimately, the attending physician or veterinarian will decide the appropriate amount and dosage regimen.

The term “patient” or “subject” includes human and non-human animals. Non-human animals include, but are not limited to, birds and mammals, in particular, mice, rabbits, cats, dogs, pigs, sheep, goats, cows, and horses.

As used herein, the phrase “pharmaceutically acceptable diluents, carriers, and/or excipients” is intended to include substances that are useful in preparing a pharmaceutical composition, and may be co-administered with an agent in accordance with the invention while allowing same to perform its intended function. These are generally safe, non-toxic, and neither biologically nor otherwise undesirable. Examples of pharmaceutically acceptable diluents, carriers, and/or excipients include solutions, solvents, dispersion media, delay agents, emulsions, and the like. Diluents, carriers, and/or excipients may contain minor amounts of additives such as substances that enhance isotonicity and chemical stability.

“Polynucleotide” (e.g., “GH”, “hGH”, “hPRL”, or “hPL”, which can be used to discuss a polynucleotide) when used in the singular or plural, generally refers to any nucleic acid sequence, e.g., any polyribonucleotide or polydeoxyribonucleotide, which may be unmodified RNA or DNA or modified RNA or DNA. This includes, without limitation, single and double stranded DNA, DNA including single and double-stranded regions, single and double stranded RNA, and RNA including single and double stranded regions, hybrid molecules comprising DNA and RNA that may be single stranded or, more typically, double stranded or include single and double stranded regions. Also included are triple stranded regions comprising RNA or DNA or both RNA and DNA. Specifically included are mRNAs, cDNAs, and genomic DNAs, and any fragments, and modifications thereof. The term includes DNAs and RNAs that contain one or more modified bases, such as tritiated bases, or unusual bases, such as inosine. The polynucleotides of the invention can encompass coding or non-coding sequences, or sense or antisense sequences, or iRNAs such as siRNAs. It will be understood that each reference to a “polynucleotide” or like term, herein, will include the full length sequences as well as any fragments, or modifications thereof.

“Polypeptide” (e.g., “GH”, “hGH”, “hPRL”, or “hPL”, which can be used to discuss a polypeptide) as used herein, refers to an oligopeptide, peptide, or protein, or fragment thereof, and to naturally occurring, recombinant, synthetic, or semi-synthetic molecules. Where “polypeptide” is recited herein to refer to an amino acid sequence of a naturally occurring protein molecule, “polypeptide” and like terms, are not meant to limit the amino acid sequence to the complete, native amino acid sequence for the full length molecule. It will be understood that each reference to a “polypeptide” or like term, herein, will include the full length sequence, as well as any fragments, or modifications thereof.

“SEQ ID NO:” as referred to herein, can indicate each sequence identifier individually, or any combination thereof, or all such sequence identifiers.

The terms “substantially purified” or “isolated” as used herein, refer to nucleic or amino acid sequences that are removed from their cellular, recombinant, synthetic, or semi-synthetic environment, and are at least 60% free, preferably 75% free, and most preferably at least 90% free or at least 99% free from other components with which they are associated in their environment. “Isolated” polynucleotides and antibodies have been identified and separated from at least one contaminant molecule with which they are associated in their natural state. Accordingly, it will be understood that isolated polynucleotides and antibodies are in a form which differs from the form or setting in which they are found in nature. It will further be appreciated that “isolated” does not necessarily reflect the exact extent (e.g., a specific percentage) to which the sequence has been purified.

“Treatment” and like terms refer to methods and compositions to prevent, cure, or ameliorate a medical disorder (e.g., medical disease, condition, or syndrome), or reduce at least a symptom of such disorder. In particular, this includes methods and compositions to prevent or delay onset of a medical disorder; to cure, correct, reduce, slow, or ameliorate the physical or developmental effects of a disorder; and/or to prevent, end, reduce, or ameliorate the pain or suffering caused the disorder. The term “treatment” to be considered in its broadest context. The term does not necessarily imply that the subject is treated until total recovery. Accordingly, “treatment” as used herein broadly includes inhibiting, reducing or preventing cell proliferation, cell survival, and/or cell motility; ameliorating the symptoms or severity of cell proliferation, cell survival, and/or cell motility; or preventing or otherwise reducing the risk of developing cell proliferation, cell survival, and/or cell motility, for example cancer, and in particular, breast cancer, colon cancer, lung cancer, prostate cancer, endometrial cancer, and endometriosis, among other conditions.

A “variant” of polypeptide, as used herein, refers to an amino acid sequence that is altered by one or more amino acids. Similarly, a variant antibody is altered by one or more amino acids. A variant polynucleotide is altered by one or more nucleotides. A variant may result in “conservative” changes, wherein a substituted amino acid has similar structural or chemical properties, e.g., replacement of leucine with isoleucine. More rarely, a variant may result in “nonconservative” changes, e.g., replacement of a glycine with a tryptophan. Analogous minor variations may also include amino acid deletions or insertions, or both. Guidance in determining which amino acid residues may be substituted, inserted, or deleted without abolishing biological or immunogenic activity may be found using computer programs well known in the art, for example, LASERGENE software (DNASTAR).

The invention also encompasses variants which retain at least one biological activity (e.g., effect on cell proliferation, cell survival, or cell motility) or immunogenic function. A preferred variant is one having at least 80%, and more preferably at least 90%, sequence identity to a disclosed sequence. A most preferred variant is one having at least 95%, at least 97%, at least 98%, or at least 99% sequence identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100. A useful alignment program is AlignX (Vector NTI).

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, Molecular Cloning: A Laboratory Manual, 2nd edition, Sambrook et al., 1989; also, Molecular Cloning: A Laboratory Manual, 3rd Edition, Sambrook et al., 2000; Oligonucleotide Synthesis, M Y Gait, ed., 1984; Animal Cell Culture, R. I. Freshney, ed., 1987; Methods in Enzymology, Academic Press, Inc.; Handbook of Experimental Immunology, 4th edition, D. M. Weir & C C. Blackwell, eds., Blackwell Science Inc., 1987; Gene Transfer Vectors for Mammalian Cells, J A M. Miller & M A P. Calos, eds., 1987; Current Protocols in Molecular Biology, FEM. Ausubel et al., eds., 1987; and PCR: The Polymerase Chain Reaction, Mullis et al., eds., 1994.

### Growth Hormone and Related Hormones

In humans, the hGH gene is part of a gene cluster composed of five structurally and functionally related genes. The pituitary specific hGH-N gene and four structurally and functionally related genes (hGH-V, hCS-A, hCS-B, and hCS-L that are expressed only in the placenta. They are arranged 5′ to 3′: hGH-N; Normal (also called hGH1), hCS-L; L for like (also known as hPL-1), hCS-A (also known as hPL-2), hGH-V; V=variant (also called hGH2) and hCS-B (also known as hPL-3). The five hGH-hCS genes are clustered in a DNA segment of approximately 50 kb located on the long arm of human chromosome 17 at bands q22-24. The genes are separated by intergenic regions of 6-13 kb which contain 29 interspersed middle repeat sequence elements of the Alu type. The cluster evolved from an ancestral gene and the process began over 3.5×10⁸ years ago. Generation of the present form of the locus involved duplications, insertion of putative control elements, and at least one gene conversion event. In humans, the five genes possess greater than 92% nucleotide sequence identity in their coding and flanking regions. Their molecular architecture is also identical; four small introns split the transcriptional units at identical positions and perfect codon colinearity exists for all of the open reading frames.

Messenger RNAs for two isoforms (designated 22 kDa and 20 kDa hGH) of hGH-N are generated by differential splicing of the primary transcription product. The 22 kDa hGH-N is the predominant form comprising 90% of pituitary gland mRNA for hGH-N. The 20 kDa hGH-N is identical to 22 kDa hGH except for deletion of amino acid residues 32-46. A 22 kDa hGH-V isoform is also generated from the placentally expressed hGH-V gene. No 20 kDa isoform of hGH-V has been detected, but an mRNA species retaining the in frame fourth intron and extending 20 codons into the fifth exon that can be translated into a 26 kDa membrane bound hGH-V isoform has been described. The nucleotide and amino acid sequence of GH from multiple other species has been delineated. Human GH shares greater sequence identity with hPL (87%) than porcine GH (73%), bovine GH, ovine GH, equine GH and rat GH (each approximately 65%) or the PRL family (approximately 27%). Only primate GHs bind to the hGH receptor. Under certain conditions hGH will also bind and activate the PRL receptor.

hGH is synthesized as a prohormone with a signal peptide at the amino terminus that is removed during secretion from the cell. The mature 22 kDa isoform of hGH-N is a 191 amino acid polypeptide and is the most abundant isoform of hGH in plasma. The half life of hGH in the human is approximately 25 minutes. The three dimensional structure of mammalian GH has also been determined. It contains four alpha helices, each of 21-30 amino acids in length. The helices are arranged in a left handed bundle orientation with an unusual up-up-down-down topology. For orientation in this unique topology GH contains long connective loop structures between the two sets of parallel helices and a shorter loop region between helix 2 and helix 3. hGH contains two disulphide bridges at Cys35-Cys165 and Cys182-Cys189. The Cys residues are component in the loop structures. The central core of the GH molecule comprises approximately 20 hydrophobic amino acids and smaller hydrophobic clusters of amino acids keep the four helix bundle stabilized.

The proximal 0.5 kb promoter of the hGH-N gene contains many of the regulatory DNA elements responsible for transcription and presumed tissue specificity of hGH-N expression. In addition to the TATA box, the proximal hGH-N gene promoter contains two binding sites for the presumed pituitary-specific transcription factor, Pit-1 (also known as GHF-1) required for expression of hGH. However, sequences as remote as 15 kb upstream of the hGH-N transcription initiation site are required for efficient expression of the hGH-N gene. The proximal hGH-N gene promoter also contains cis-acting elements modulating transcription in response to hormonal signals.

The GH receptor (GHR) consists of two domains (amino acids 1-123 and amino acids 128-238) that are linked by four residues (124-127) of the polypeptide chain. Each domain contains seven beta-strands arranged to form a sandwich of two antiparallel beta-sheets. The GH receptor also contains three disulphide bonds between Cys38-Cys48, Cys83-Cys94, and Cys108-Cys122. Hydrogen bonding between Arg43 and Glu 169 and a salt bridge between Arg39 and Asp123 also aid to stabilize the receptor. The expected molecular mass of the GH receptor from the amino acid sequence is 70 kDa. Posttranslational modifications such as glycosylation and ubiquitination result in a receptor mass of 100-130 kDa. The GH receptor contains five N-linked glycosylation sites with each adding approximately 10 kDa to the receptor. The GH receptor possesses 19 potential ubiquitination sites and is polyubiquitinated on multiple lysine residues. Ubiquitination of the receptor is increased by ligand binding and is required for GH receptor internalization.

GH receptor expression has been demonstrated in most organ systems including the gastrointestinal tract, male and female reproductive systems, musculoskeletal system, cardiorespiratory system, hematopoietic and immune systems, central nervous system, the integument, renal and urinary systems, and the endocrine system. Within these systems the GH receptor is expressed on both differentiated and non-differentiated cell types. The GH receptor is also expressed in cells derived from the ectoderm, mesoderm, and endoderm of the developing fetus. GHBP in the human is generated by limited proteolysis of the membrane bound GH receptor by a member of the metalloprotease family. Up to 60% of circulating GH is bound to GHBP. GHBP increases the half-life of GH in the serum by decreasing the rate of clearance and subsequent degradation. In rats, mice, and monkeys, GHBP is generated by alternative splicing of the GH receptor precursor mRNA. Some short but membrane-anchored isoforms of the GH receptor have been described in humans.

One hGH molecule interacts with the extracellular domains of two receptors leading to receptor homodimerization. hGH possesses two receptor binding sites; a high affinity site “site 1” and a lower affinity site “site 2” that sequentially interact with binding ‘pockets’ in two discrete receptor molecules. Contact with hGH at site 2 and a dimerization interface of approximately 500 Å between the two extracellular domains of the receptor stabilize the binding of the second receptor to the complex. In the GH receptor, the two-ligand binding sites are largely composed of the same amino acid residues and possess similar overall shape and structure. The amino acid residues that form sites one and two in the GHBP are located in six clusters between residues 43 and 218. The resulting receptor dimerization presumably provides the signal to generate the biological response. hGH mutated at site 2 has formed the basis for the development of effective GH receptor antagonists.

PRL is a 23-kDa protein comprising 199 amino acids in four anti-parallel alpha helices with three disulfide loops. The location of the loops is conserved but the primary sequence varies among species. Posttranslational modifications, such as glycosylation, phosphorylation, cleavage and polymerization, generate molecular heterogeneity (Sinha, Y. N. (1995) Structural variants of prolactin: occurrence and physiological significance. Endocr. Rev. 16, 354-369). Human PRL (hPRL) is N-glycosylated on Asp31, with both glycosylated and non glycosylated forms circulating at variable ratios. Glycosylated PRL has a lower binding affinity to the PRL receptor and a reduced activity in some bioassays, whereas phosphorylated PRL binds well to the receptor but might act as an antagonist (Xu, X. et al. (2001) A molecular mimic of phosphorylated prolactin markedly reduced tumor incidence and size when DU145 human prostate cancer cells were grown in nude mice. Cancer Res. 61, 6098-6104).

A cleaved form of PRL (16K PRL) has anti-angiogenic properties (Struman, I. et al. (1999) Opposing actions of intact and N-terminal fragments of the human prolactin/growth hormone family members on angiogenesis: an efficient mechanism for the regulation of angiogenesis. Proc. Natl. Acad. Sci. U.S.A. 96, 1246-1251). Polymerization and conjugation to IgG can form large molecular species; ‘big’ PRL (50-60 kDa) and macro-PRL (150-170 kDa) are present in serum of patients with hyperprolactinemia. hPRL, but not PRL or growth hormone (GH) from other species, binds to heparin (Khurana, S. et al. (1999) Heparin-binding property of human prolactin: a novel aspect of prolactin biology. Endocrinology 140, 1026-1029).

PRL is synthesized in multiple extrapituitary sites, including the decidua, myometrium, breast, prostate, brain and immune cells (Ben-Jonathan, N. et al. (1996) Extrapituitary prolactin: distribution, regulation, functions and clinical aspects. Endocr. Rev. 17, 639-669). Uptake and retention from the circulation is another distinct feature of PRL. Uptake can be used for transporting PRL into fluid compartments such as cerebrospinal fluid and milk (Ollivier-Bousquet, M. et al. (1993) Prolactin transit through mammary epithelial cells and appearance in milk. Endocr. Regul. 27, 115-124), whereas PRL retention by the extracellular matrix can increase its concentration in the vicinity of responsive cells. PRL is also internalized within target cells (Rycyzyn, M. A. et al. (2000) Role of cyclophilin B in prolactin signal transduction and nuclear retrotranslocation. Mol. Endocrinol. 14, 1175-1186), although the exact function(s) of intracellular PRL or the potential for its recycling and exocytosis are unclear.

As to structural homology, there is 85% sequence identity shared by the peptide sequences of hGH and hPL, while hPRL shares approximately 25% similarity with the other two hormones (reviewed by A M Corbacho et al., Journal of Endocrinology, 2002, 173, 219-238). Although, hPL has show relatively low affinity for GHR (Lowman et al. 1991), all three human hormones bind with high affinity to the PRL receptor (Nicoll et al. 1986, supra, Goffin et al. 1996b, supra). In addition, all three hormones include between 190-200 amino acids and the mature proteins have a molecular mass of ˜22-23 kDa. Their tertiary structure is stabilized by intra-chain disulfide bonds and is basically composed of four anti-parallel alpha-helices (for reviews see Goffin et al. 1996b, supra, Bole-Feysot C, Goffin V, Edery M, Binart N & Kelly P A 1998 Prolactin (PRL) and its receptor: actions, signal transduction pathways and phenotypes observed in PRL receptor knockout mice. Endocrine Reviews 19 225-268). Moreover, PRL and GH receptors are structurally and functionally related to members of the class 1 superfamily of cytokine receptors (Bazan F 1989 A novel family of growth factor receptors: a common binding domain in the growth hormone, prolactin, erythropoietin and IL-6 receptors, and the p75 IL-2 receptor beta-chain. Biochemical and Biophysical Research Communications 164 788-795, Kelly P A, Djiane J, Postel-Vinay M C & Edery M 1991 The prolactin/growth hormone receptor family. Endocrine Reviews 12 235-251, Cosman D 1993 The hematopoietin receptor superfamily. Cytokine 5 95-106).

These hormone receptors are transmembrane proteins that share highly conserved sequences in their extracellular and intracellular domains (reviewed by A M Corbacho et al., Journal of Endocrinology, 2002, 173, 219-238; see, also, Murakami M, Narazaki M, Hibi M, Yawata H, Yasukawa K, Hamaguchi M, Taga T & Kishimoto T 1991 Critical cytoplasmic region of the interleukin 6 signal transducer gp130 is conserved in the cytokine receptor family. PNAS 88 11349-11353 1991, Cosman 1993, supra, O'Neal K D & Yu-Lee L-Y 1993 The proline-rich motif (PRM): a novel feature of the cytokine/hematopoietin receptor superfamily. Lymphokine and Cytokine Research 12 309-312, Bole-Feysot et al. 1998, supra, Waters M J, Shang C A, Behncken S N, Tam S-P, Li H, Shen B & Lobie P E 1999 Growth hormone as a cytokine. Clinical and Experimental Pharmacology and Physiology 26 760-764), and they all can activate the JAK/STAT (Janus kinases/signal transducers and activators of transcription) signal transduction pathway as a consequence of ligand binding-induced homodimerization of the receptors (Ihle J N & Kerr I M 1995 Jaks and Stats in signaling by the cytokine receptor superfamily. Trends in Genetics 11 69-74, Yu-Lee L-Y 1997 Molecular actions of prolactin in the immune system. Proceedings of the Society for Experimental Biology and Medicine 215 35-52., Bole-Feysot et al. 1998, supra, Waters et al. 1999, supra).

### Growth Hormones and Cancer

Recently, a number of publications have described the association of growth hormone with cancer both in humans and animal models. Although transgenic mouse models clearly support a role for GH in cancer, corresponding clinical evidence in human neoplasia has harder to come by and more contentious. Increased circulating hGH is known to increase expression of IGF-1 which has been linked to risk of malignancy in numerous studies (C. Laban, S. A. Bustin, P. J. Jenkins, The GH-IGF-I axis and breast cancer, Trends Endocrinol. Metab. 14 (2003) 28-34; H. M. Khandwala, I. E. McCutcheon, A. Flyvbjerg, K. E. Friend, 892 The effects of insulin-like growth factors on tumourigenesis and neoplastic growth, Endocr. Rev. 21 (2000) 215-244. 894). However, no change in hGH serum levels was seen in a study of premenopausal women with breast cancer (R. R. Love, D. R. Rose, T. S. Surawicz, P. A. Newcomb, Prolactin and growth hormone levels in premenopausal women with breast cancer and healthy women with a strong family history of breast cancer, Cancer 68 (1991) 1401-1405).

Moreover, in a recent study, serum concentrations of hGH were found to inversely correlate with risk of prostate cancer (B. Fuhrman, M. Barba, H. J. Schunemann, T. Hurd, T. Quattrin, R. Cartagena, et al., Basal growth hormone concentrations in blood and the risk for prostate cancer: a case-control study, Prostate 64 (2005) 109-115). It has been suggested that this inverse association may be due to a negative feedback loops on hGH secretion generated by IGF-1 produced by the tumour (S. Yakar, D. Leroith, P. Brodt, The role of the growth hormone/insulin-like growth factor axis in tumor growth and progression: lessons from animal models, Cytokine Growth Factor Rev. 16 (2005) 407-420; R. R. Love, D. R. Rose, T. S. Surawicz, P. A. Newcomb, Prolactin and growth hormone levels in premenopausal women with breast cancer and healthy women with a strong family history of breast cancer, Cancer 68 (1991) 1401-1405).

Neither the full spectrum of PRL functions in humans nor its involvement in carcinogenesis is well understood. Regarding breast cancer, PRL administration increases the incidence, size and number of spontaneous and virus-induced mammary tumours, and sustains carcinogen-induced tumor growth in rodents (Nandi, S. et al. (1995) Hormones and mammary carcinogenesis in mice, rats, and humans: a unifying hypothesis. Proc. Natl. Acad. Sci. U.S.A. 92, 3650-3657). Moreover, transgenic mice overexpressing the gene encoding PRL develop mammary tumours (Wennbo, H. et al. (1997) Activation of the prolactin receptor but not the growth hormone receptor is important for induction of mammary tumours in transgenic mice. J. Clin. Invest. 100, 2744-2751). Yet, an association between the circulating levels of PRL and breast cancer in humans is unclear.

The connection between PRL and endometrial neoplasms is also unclear. Serum PRL is elevated in a subpopulation of women with endometriosis, but is unchanged in patients with endometrial cancer (Gregoriou, G. et al. (1999) Evaluation of serum prolactin levels in patients with endometriosis and infertility. Gynecol. Obstet. Invest. 48, 48-51). However, an immortalized human endometrial stromal cell line, N5, expresses PRL and responds to estrogen and progesterone (Brar, A. K. et al. (1999) N5 endometrial stromal cell line: a model system to study decidual prolactin gene expression. In Vitro Cell. Dev. Biol. Anim. 35, 150-154).

Regarding prostate cancer, the PRL receptor is expressed in fetal, prepubertal, and adult human prostate epithelial cells (Leav, I. et al. (1999) Prolactin receptor expression in the developing human prostate and in hyperplastic, dysplastic, and neoplastic lesions. Am. J. Pathol. 154, 863-870). It is also detected in benign prostatic hyperplasia and its expression is higher in dysplasia, but lower in higher-grade carcinomas. Among cancer cell lines, the PRL receptor is expressed by LNCaP cells and PC-3 cells (Peirce, S. K. et al. (2001) Quantification of prolactin receptor mRNA in multiple human tissues and cancer cell lines by real time RT PCR. J. Endocrinol. 171, R1-R443 Leav, I. et al. (1999) Prolactin receptor expression in the developing human prostate and in hyperplastic, dysplastic, and neoplastic lesions. Am. J. Pathol. 154, 863-870). Yet, PRL stimulates proliferation of DU145 and PC3 cells but has no effects on LNCaP cells (Janssen, T. et al. (1996) In vitro characterization of prolactin-induced effects on proliferation in the neoplastic LNCaP, DU145, and PC3 models of the human prostate. Cancer 77, 144-149). Thus, there is an ongoing need for data and reagents pertaining to growth hormone and its role in human cancers.

Consistent with the findings disclosed herein, hGH is highly or moderately expressed in several cancer cell lines, including cell lines for endometrial cancer (see, Examples). Moreover, endometrial cancer cells overexpressing hGH grow significant faster than control cells stably transfected by empty vector, as shown by total cell number. BrdU incorporation assays show that the increase in total cell number is the result of increased cell proliferation. Measurement of apoptosis shows that autocrine hGH also acts to increase total cell number by increasing cell survival. Further, the increase in cell proliferation and survival can be effectively reversed by targeting hGH using anti-hGH antibodies. Such antibodies inhibit the function of autocrine hGH, resulting in a reduction in growth rate and inhibition of cell proliferation and cell survival (see, Examples).

Traditional therapies used to treat cancer patients include surgery, radiation, chemotherapy, and sex hormone therapy. However, there are always a large number of cancer patients whose condition does not respond to these traditional therapies. Because uncontrolled cell proliferation, cell survival, and/or cell motility are the hallmarks of cancer, for many decades, the most direct and effective strategy in the field of cancer therapy has been to attempt to stop cancer growth by blocking cell division. Traditional chemotherapy agents are therefore toxic to healthy cells as well as cancer cells. Moreover, the inherent genetic instability of neoplastic cells eventually results in the selection of drug-resistant clones after prolonged exposure to chemotherapy.

The inhibitory agents of the present invention provide for novel compositions and methods for treating, preventing, and/or inhibiting cancer growth by targeting specific genes involved in cell growth and proliferation. In particular, the inhibitory agents of the present invention provide of novel compostions and methods for treating, preventing and/or inhibiting cancer growth by targeting human growth hormone, and related hormones. The antibodies of the invention are predicted to avoid the problems which affect other known inhibitory agents, including reduced specificity, lack of long-term efficacy, and deleterious immune responses. The antibodies are also predicted to provide superior enhancement for chemotherapeutic agents. In particular, the antibodies would have increased stability compared, for example, to peptide reagents. In addition, the hGH antibodies bind to hGH, and not the receptor, and therefore cannot provide a stimulatory signal (i.e., agonist or partial agonist activity). The inventors have observed the effectiveness of antibody inhibition of autocrine hGH, as opposed to endocrine hGH. As such, the disclosed inhibitors (e.g., antibodies and polynucleotides) are superior for treatment of tumours which secrete their own hGH. Such inhibitors are preferred for treatment of solid tumours as they would not necessarily need to penetrate to the interior of the tumour and or to bind to the receptor on the cell surface, but could work by binding soluble hGH in the tumour environment. The inhibitors can thereby be used in other cancers which produce or are responsive to GH and related hormones, for example, for breast cancer, colon cancer, lung cancer, prostate cancer, endometrial cancer, and endometriosis. Similarly, siRNA can be used to reduce hormone expression in these cancers, and thereby provide treatment.

Thus, the data shown herein demonstrate that hGH promotes the proliferation and survival of endometrial carcinoma cells, and that this proliferation and survival can be effectively inhibited by inhibiting the function of autocrine hGH via antibody binding. As such, GH and related hormones, particularly hGH and variants thereof, hPRL, and hPL, represent ideal novel targets for cancer treatment and diagnosis, especially for endometrial cancer or endometriosis, or breast cancer, colon cancer, lung cancer, or prostate cancer. Any reagents that inhibit the biological activity (e.g., autocrine activity) of these hormones can be used to inhibit the proliferation, survival, and/or motility of cancer cells. These reagents can include, for example, chemical compounds (e.g., small molecules), antagonists, antibodies, and iRNAs. Similarly, diagnostic agents (e.g., polynucleotides and antibodies) can be used to determine the presence or levels of GH and/or related hormones, particularly hGH and/or variants thereof, hPRL, or hPL, and detect a cancerous condition, e.g., cancer onset, progression, or recurrence.

Isolated GH and related hormones, particularly hGH or variants thereof, hPRL, or hPL, may find various applications. For example, the hormones may be used as controls in diagnostic assays or in kits as described herein. Alternatively, the hormones may be used to generate further antibodies of therapeutic or diagnostic use. The hormones may also be used as a substrate to study the processes of cell proliferation, cell survival, and cell motility, and other cellular mechanisms.

### Polynucleotides and Polypeptides

The invention encompasses the use of polypeptides of GH and/or a related hormone, particularly hGH or variants thereof, hPRL, or hPL, including those comprising at least one of SEQ ID NO: 1-26, and fragments, and modifications thereof. The invention also encompasses the use of these polypeptides in the diagnosis of cancer, especially endometrial cancer or endometriosis, or breast cancer, colon cancer, lung cancer, or prostate cancer. The invention further encompasses the use of the polypeptides for preparing antibodies to inhibit the cell proliferation, cell survival, or cell motility of such cells.

The polypeptides as used herein comprise at least one sequence selected from the group consisting of: (a) polypeptides comprising at least one amino acid sequence selected from the group consisting of SEQ ID NO: 1-26, or fragments, or modifications thereof; (b) polypeptides comprising a functional domain of at least one amino acid sequence selected from the group consisting of SEQ ID NO: 1-26, and fragments and modifications thereof; and (c) polypeptides comprising at least a specified number of contiguous residues of at least one amino acid sequence selected from the group consisting of SEQ ID NO: 1-26, or modifications thereof. In one particular embodiment, the invention encompasses an isolated polypeptide of GH or a related hormone, particularly hGH or variants thereof, hPRL, or hPL, comprising the amino acid sequence of at least one of SEQ ID NO: 1-26. All of these sequences are collectively included to herein as polypeptides for use with the invention.

The polypeptides may be expressed and used in various assays to detect or measure the activity and/or levels of the polypeptides. The polypeptides may be used for large-scale synthesis and isolation protocols, for example, for commercial production. Such polypeptides may be used to raise antibodies, to isolate corresponding amino acid sequences, and to quantitatively determine levels of the amino acid sequences.

The invention encompasses the use of polynucleotides of GH and/or a related hormone, particularly hGH or variants thereof, hPRL, or hPL, including those of SEQ ID NO: 27-96, and fragments, and modifications thereof. The invention also encompasses the use of these polynucleotides in the diagnosis of cancer, especially breast cancer, colon cancer, lung cancer, prostate cancer, endometrial cancer, or endometriosis. The invention further encompasses the use of these polynucleotides for the inhibition of cell proliferation, cell survival, or cell motility of such cells. Accordingly, the invention encompasses the use of these polynucleotides for preparing expression vectors and host cells, and for preparing antisense polynucleotides and iRNAs.

The polynucleotides as used herein comprise at least one sequence selected from the group consisting of: (a) sequences comprising a coding sequence for at least one amino acid sequence selected from the group consisting of SEQ ID NO: 1-26, or fragments, or modifications thereof; (b) complements, reverse sequences, and reverse complements of a coding sequence for at least one amino acid sequence selected from the group consisting of SEQ ID NO: 1-26, or fragments, or modifications thereof; (c) open reading frames contained in the coding sequence for at least one amino acid sequence selected from the group consisting of SEQ ID NO: 1-26, or their fragments, or modifications (d) functional domains of a coding sequence for at least one amino acid sequence selected from the group consisting of SEQ ID NO: 1-26, or fragments, or modifications thereof; (e) sequences comprising at least a specified number of contiguous residues of a coding sequence for at least one amino acid sequence selected from the group consisting of SEQ ID NO: 1-26, or modifications thereof; and (f) sequences comprising at least a specified number of contiguous nucleotides of SEQ ID NO: 27-96, or modifications thereof. In one particular embodiment, the invention encompasses an isolated polynucleotide comprising a coding sequence for at least one amino acid sequence selected from the group consisting of SEQ ID NO: 1-26. In another particular embodiment, the invention uses an isolated polynucleotide comprising a sequence selected from the group consisting of SEQ ID NO: 27-96. Oligonucleotide probes and primers and their modifications can also be used. All of these polynucleotides and oligonucleotide probes and primers are collectively included, as polynucleotides for use with the invention.

The isolated polynucleotides can also be used in genome mapping, in physical mapping, and in cloning of genes of more or less related species. Probes designed using the polynucleotides may be used to detect the presence and examine the expression patterns of genes in any organism having sufficiently homologous DNA and RNA sequences in their cells, employing techniques that are well known in the art, such as slot blot techniques or microarray analysis. Primers designed using the polynucleotides may be employed for sequencing and PCR amplifications. The polynucleotides may also be used as compositions, for example, pharmaceutical compositions. The polynucleotides can also be used to provide health benefits. For such benefits, the polynucleotides can be presented as expression vectors or host cells comprising expression vectors.

It will be appreciated by those skilled in the art that as a result of the degeneracy of the genetic code, a multitude of nucleotide sequences encoding the polypeptides of the invention, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to naturally occurring amino acid sequences, and all such variations are to be considered as being specifically disclosed.

Nucleotide sequences which encode the polypeptides, or their fragments, or modifications, are preferably capable of hybridizing to the nucleotide sequence of the naturally occurring sequence under appropriately selected conditions of stringency. However, it may be advantageous to produce nucleotide sequences encoding a polypeptide, or its fragment, or modification, possessing a substantially different codon usage. Codons may be selected to increase the rate at which expression of the polypeptide occurs in a particular prokaryotic or eukaryotic host in accordance with the frequency with which particular codons are utilized by the host. Other reasons for substantially altering the nucleotide sequence without altering the encoded amino acid sequences include the production of RNA transcripts having more desirable properties, such as a greater half-life, than transcripts produced from the naturally occurring sequence.

The invention also encompasses production of polynucleotides, or fragments, or modifications thereof, entirely by synthetic chemistry. After production, the synthetic sequence may be inserted into any of the many available expression vectors and cell systems using reagents that are well known in the art. Moreover, synthetic chemistry may be used to introduce mutations into a sequence encoding a polypeptide, or any fragments, or modifications thereof. Also encompassed by the invention are polynucleotide sequences that are capable of hybridizing to the claimed nucleotide sequences, and in particular, those shown in SEQ ID NO: 27-96, under various conditions of stringency as taught in Wahl, G. M. and S. L. Berger (1987; Methods Enzymol. 152:399-407) and Kimmel, A. R. (1987; Methods Enzymol. 152:507-511).

Methods for DNA sequencing which are well known and generally available in the art may be used to practice any of the embodiments of the invention. The methods may employ such enzymes as the Klenow fragment of DNA polymerase I, SEQUENASE (U.S. Biochemical Corp, Cleveland, Ohio), Taq polymerase (Perkin Elmer), thermostable T7 polymerase (Amersham Pharmacia Biotech, Piscataway, N.J.), or combinations of polymerases and proofreading exonucleases such as those found in the ELONGASE Amplification System (Life Technologies, Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) the ABI Catalyst and 373 and 377 DNA Sequencers (Perkin Elmer), or the Genome Sequencer 20™ (Roche Diagnostics).

The nucleic acid sequences encoding the polypeptides may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Capillary electrophoresis systems which are commercially available may be used to analyze the size or confirm the nucleotide sequence of sequencing or PCR products. In particular, capillary sequencing may employ flowable polymers for electrophoretic separation, four different fluorescent dyes (one for each nucleotide), which are laser activated, and detection of the emitted wavelengths by a charge coupled device camera. Output/light intensity may be converted to electrical signal using appropriate software (e.g., GENOTYPER and Sequence NAVIGATOR, Perkin Elmer) and the entire process from loading of samples to computer analysis and electronic data display may be computer controlled. Capillary electrophoresis is especially preferable for the sequencing of small pieces of DNA which might be present in limited amounts in a particular sample.

In another embodiment, polynucleotides or fragments thereof which encode polypeptides of the invention may be used in recombinant DNA molecules to direct expression of the polypeptides, or fragments, or modifications thereof, in appropriate host cells. Due to the inherent degeneracy of the genetic code, other DNA sequences which encode substantially the same or a functionally equivalent amino acid sequence may be produced, and these sequences may be used to clone and express the polypeptides. The nucleotide sequences of the present invention can be engineered using methods generally known in the art in order to alter amino acid encoding sequences for a variety of reasons, including but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, introduce mutations, and so forth.

In another embodiment of the invention, a natural, modified, or recombinant nucleic acid sequence encoding a polypeptide may be ligated to a heterologous sequence to encode a fusion protein. For example, it may be useful to encode a chimeric sequence that can be recognized by a commercially available antibody. A fusion protein may also be engineered to contain a cleavage site located between the polypeptide of the invention and the heterologous protein sequence, so that the polypeptide may be cleaved and purified away from the heterologous moiety.

In another embodiment, sequences encoding polypeptides may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the polypeptide itself may be produced using chemical methods to synthesize the amino acid sequence, or a fragment thereof. For example, polypeptide synthesis can be performed using various solid-phase techniques (Roberge, J. Y. et al. (1995) Science 269:202-204; Merrifield J. (1963) J. Am. Chem. Soc. 85:2149-2154) and automated synthesis may be achieved, for example, using the ABI 431A Peptide Synthesizer (Perkin Elmer). Various fragments of polypeptides may be chemically synthesized separately and combined using chemical methods to produce the full length molecule.

The newly synthesized polypeptide may be isolated by preparative high performance liquid chromatography (e.g., Creighton, T. (1983) Proteins Structures and Molecular Principles, WH Freeman and Co., New York, N.Y.). The composition of the synthetic polypeptides may be confirmed by amino acid analysis or sequencing (e.g., the Edman degradation procedure; Creighton, supra). Additionally, the amino acid sequence of the polypeptide, or any part thereof, may be altered during direct synthesis and/or combined using chemical methods with sequences from other proteins, or any part thereof, to produce a modified molecule.

In order to express a biologically active polypeptides, the nucleotide sequences encoding the polypeptide or functional equivalents, may be inserted into appropriate expression vector, e.g., a vector which contains the necessary elements for the transcription and translation of the inserted coding sequence. Methods which are well known to those skilled in the art may be used to construct expression vectors containing sequences encoding the polypeptide and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Laboratory Press, Plainview, N.Y.; also, Sambrook, J. et al. (2000) Molecular Cloning, A Laboratory Manual, Cold Spring Harbor Laboratory Press, Plainview, N.Y.; and Ausubel, F. M. et al. (1989) Current Protocols in Molecular Biology, John Wiley & Sons, New York, N.Y.

A variety of expression vector/host systems may be utilized to contain and express sequences encoding the polypeptides of the invention. These include, but are not limited to, microorganisms such as bacteria transformed with recombinant phage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems. For bacteria, useful plasmids include pET, pRSET, pTrcHis2, and pBAD plasmids from Invitrogen, pET and pCDF plasmids from Novagen, and Director™ plasmids from Sigma-Aldrich. In particular, E. coli can be used with the expression vector pET. The invention is not limited by the expression vector or host cell employed.

The “control elements” or “regulatory sequences” are those non-translated regions (e.g., enhancers, promoters, 5′ and 3′ untranslated regions) which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector.

In bacterial systems, a number of expression vectors may be selected depending upon the use intended for the polypeptide. For example, when large quantities of polypeptide are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding a polypeptide may be ligated into the vector in frame with sequences for the amino-terminal Met and the subsequent 7 residues of β-galactosidase so that a hybrid protein is produced; pIN vectors (Van Heeke, G. and S. M. Schuster (1989) J. Biol. Chem. 264:5503-5509); and the like.

pGEX vectors (Promega, Madison, Wis.) may also be used to express the polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor Xa protease cleavage sites so that the cloned polypeptide of interest can be released from the GST moiety at will. In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

Specific initiation signals may also be used to achieve more efficient translation of sequences encoding the polypeptides of the invention. Such signals include the ATG initiation codon and adjacent sequences. In cases where sequences encoding a polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell may be chosen for its ability to modulate the expression of the inserted sequences or to process the expressed polypeptide in the desired fashion. Such modifications of the sequence include, but are not limited to, acetylation, carboxylation, glycosylation, phosphorylation, lipidation, and acylation. Post-translational processing which cleaves a “prepro” form of the polypeptide may also be used to facilitate correct insertion, folding, and/or function. Different host cells which have specific cellular machinery and characteristic mechanisms for post-translational activities are available from the American Type Culture Collection (ATCC; Bethesda, Md.) and may be chosen to ensure the correct modification and processing of the sequence. Specific host cells include, but are not limited to, Rhodotorula, Aureobasidium, Saccharomyces, Sporobolomyces, Pseudomonas, Erwinia and Flavobacterium; or such other organisms as Escherichia, Lactobacillus, Bacillus, Streptomyces, and the like. Particular host cells include Escherichia coli, which is particularly suited for use with the present invention, Saccharomyces cerevisiae, Bacillus thuringiensis, Bacillus subtilis, Streptomyces lividans, and the like.

There are several techniques for introducing nucleic acids into eukaryotic cells cultured in vitro. These include chemical methods (Felgner et al., Proc. Natl. Acad. Sci., USA, 84:7413 7417 (1987); Bothwell et al., Methods for Cloning and Analysis of Eukaryotic Genes, Eds., Jones and Bartlett Publishers Inc., Boston, Mass. (1990), Ausubel et al., Short Protocols in Molecular Biology, John Wiley and Sons, New York, N.Y. (1992); and Farhood, Annal. NY Acad. Sci., 716:23 34 (1994)), use of protoplasts (Bothwell, supra) or electrical pulses (Vatteroni et al., Muth. Res., 291:163 169 (1993); Sabelnikov, Prog. Biophys. Mol. Biol., 62: 119 152 (1994); Bothwell et al., supra; and Ausubel et al., supra), use of attenuated viruses (Davis et al., J. Virol. 1996, 70(6), 3781 3787; Brinster et al. J. Gen. Virol. 2002, 83(Pt 2), 369 381; Moss, Dev. Biol. Stan., 82:55 63 (1994); and Bothwell et al., supra), as well as physical methods (Fynan et al., supra; Johnston et al., Meth. Cell Biol., 43(Pt A):353 365 (1994); Bothwell et al., supra; and Ausubel et al., supra).

Successful delivery of nucleic acids to animal tissue can be achieved by cationic liposomes (Watanabe et al., Mol. Reprod. Dev., 38:268 274 (1994)), direct injection of naked DNA or RNA into animal muscle tissue (Robinson et al., Vacc., 11:957 960 (1993); Hoffman et al., Vacc. 12:1529 1533; (1994); Xiang et al., Virol., 199:132 140 (1994); Webster et al., Vacc., 12:1495 1498 (1994); Davis et al., Vacc., 12:1503 1509 (1994); Davis et al., Hum. Molec. Gen., 2:1847 1851 (1993); Dalemans et al. Ann NY Acad. Sci. 1995, 772, 255 256. Conry, et al. Cancer Res. 1995, 55(7), 1397-1400), and embryos (Naito et al., Mol. Reprod. Dev., 39:153 161 (1994); and Burdon et al., Mol. Reprod. Dev., 33:436 442 (1992)), intramuscular injection of self replicating RNA vaccines (Davis et al., J. Virol. 1996, 70(6), 3781 3787; Balasuriya et al. Vaccine 2002, 20(11 12), 1609 1617) or intradermal injection of DNA using “gene gun” technology. (Johnston et al., supra).

A variety of protocols for detecting and measuring the expression of the polypeptides of the invention, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay can be used with monoclonal antibodies reactive to two non-interfering epitopes on the polypeptide, but a competitive binding assay can also be used. These and other assays are described, among other places, in Hampton, R. et al. (1990; Serological Methods, a laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences encoding the polypeptides, or any fragments, or modifications thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits Amersham Pharmacia Biotech, Promega, and US Biochemical. Suitable reporter molecules or labels, which may be used for ease of detection, include radionuclides, enzymes, fluorescent, chemiluminescent, or chromogenic agents as well as substrates, cofactors, inhibitors, magnetic particles, and the like.

Expression vectors or host cells transformed with expression vectors may be cultured under conditions suitable for the expression and recovery of the polypeptide from culture. The culture can comprise components for in vitro or in vivo expression. In vitro expression components include those for rabbit reticulocyte lysates, E. coli lysates, and wheat germ extracts, for example, Expressway™ or RiPs systems from Invitrogen, Genelator™ systems from iNtRON Biotechnology, EcoPro™ or STP3™ systems from Novagen, TNT Quick Coupled systems from Promega, and EasyXpress systems from QIAGEN. The polypeptide produced from culture may be secreted or contained intracellularly depending on the sequence and/or the vector used. In particular aspects, expression vectors which encode a phage polypeptide can be designed to contain signal sequences which direct secretion of the polypeptide through a prokaryotic or eukaryotic cell membrane.

Other constructions may include an amino acid domain which will facilitate purification of the polypeptide. Such domains include, but are not limited to, metal chelating peptides such as histidine-tryptophan (e.g., 6×-HIS) modules that allow purification on immobilized metals, protein A domains that allow purification on immobilized immunoglobulin, and the domain utilized in the FLAG® extension/affinity purification system (Immunex Corp., Seattle, Wash.). Useful epitope tags include 3X-FLAG®, HA, VSV-G, V5, HSV, GST, GFP, MBP, GAL4, and β-galactosidase. Useful plasmids include those comprising a biotin tag (e.g., PinPoint™ plasmids from Promega), calmodulin binding protein (e.g., pCAL plasmids from Stratagene), streptavidin binding peptide (e.g., InterPlay™ plasmids from Stratagene), a c-myc or FLAG® tag (e.g., Immunoprecipitation plasmids from Sigma-Aldrich), or a histidine tag (e.g., QIAExpress plasmids from QIAGEN).

To facilitate purification, expression vectors can include a cleavable linker sequences such as those specific for Factor Xa or enterokinase (Invitrogen, San Diego, Calif.). For example, the vector can include one or more linkers between the purification domain and the polypeptide. One such expression vector provides for expression of a fusion protein comprising a polypeptide of the invention and a nucleic acid encoding 6 histidine residues preceding a thioredoxin or an enterokinase cleavage site. The histidine residues facilitate purification on IMAC (immobilized metal ion affinity chromatography as described in Porath, J. et al. (1992) Prot. Exp. Purif. 3: 263-281) while the enterokinase cleavage site provides a means for purifying the polypeptide from the fusion protein. A discussion of vectors which contain fusion proteins is provided in Kroll, D. J. et al. (1993; DNA Cell Biol. 12:441-453).

Polynucleotides for Inhibition of GH and/or Related Hormones

As mentioned herein, in one embodiment, polynucleotides may be utilised to inhibit of GH and/or a related hormone, particularly hGH and/or variants thereof, hPRL, or hPL, in accordance with the invention. Such polynucleotides may be DNA, RNA, single stranded, or double stranded. Polynucleotides for use with the invention may be referred to herein as “isolated” polynucleotides. Isolated polynucleotides may be obtained using a number of techniques known in the art. For example, recombinant DNA technology may be used as described for example in Sambrook, J. et al. (1989) Molecular Cloning: A Laboratory Manual (2nd edition), Cold Spring Harbor Laboratory Press, Plainview, N.Y. and Sambrook and Russell (2000) Molecular Cloning: A Laboratory Manual (3rd Edition), Cold Spring Harbor Laboratory Press, Plainview, N.Y. Similarly, chemical synthesis, for example, using phosphoramidite and solid phase chemistry, may be used. Polynucleotides may be designed on the basis of the disclosed nucleic acid sequence data, the known relative interactions between nucleotide bases, known sequence homology, and the particular nucleic acid technology to be employed, as may be exemplified herein after.

In one embodiment, interfering RNAs (iRNAs or siRNAs) may be utilised to inhibit expression of GH and/or a related hormone, particularly hGH and/or variants thereof, hPRL, or hPL. Polynucleotides of use in iRNA techniques will typically have 100% complementarity to their target. However, it should be appreciated that this need not be the case, provided the iRNA retains specificity for its target and the ability to block translation. Exemplary iRNA molecules may be in the form of ˜18 to 21 by double stranded RNAs with 3′ dinucleotide overhangs, although shorter or longer molecules may be appropriate. In cases where the iRNA is produced in vivo by an appropriate nucleic acid vector, it will typically take the form of an RNA molecule having a stem-loop structure, for example, an approximately 19 nucleotide stem and a 9 nucleotide loop with 2-3 Us at the 3′ end. Algorithms of use in designing siRNA are available from Cenix (Dresden, Germany, via Ambion, Tex.).

Exemplary siRNA molecules can include the following sequences:

In one aspect, the iRNA comprises a nucleotide sequence selected from the group consisting of:

| | |
|---|---|
| 5′-GAGGGCAGTGCCTTCCCAA-3′ | (SEQ ID NO: 33) |
| 5′-GCCTATATCCCAAAGGAAC-3′ | (SEQ ID NO: 34) |
| 5′-CCTGGTGTACGGCGCCTCT-3′ | (SEQ ID NO: 35) |
| 5′-CTGACAGCAACGTCTATGA-3′ | (SEQ ID NO: 36) |
| 5′-GTATTCATTCCTGCAGAAC-3′ | (SEQ ID NO: 37) |
| 5′-CAGCCTGGTGTACGGCGCC-3′ | (SEQ ID NO: 38) |
| 5′-CGATGACGCACTACTCAAG-3′ | (SEQ ID NO: 39) |

In one preferred aspect, the iRNA comprises a nucleotide sequence selected from the group consisting of:

| | |
|---|---|
| 5′-GAGGGCAGTGCCTTCCCAATTCAAGAGATTGGGAAGGCACTGCCCTC-3′ | (SEQ ID NO: 40) |
| \|---Sense Strand---\|--Loop--\|-Antisense Strand-\| | |
| 5′-GCCTATATCCCAAAGGAACTTCAAGAGAGTTCCTTTGGGATATAGGC-3′ | (SEQ ID NO: 41) |
| \|---Sense Strand---\|--Loop--\|-Antisense Strand-\| | |
| 5′-CCTGGTGTACGGCGCCTCTTTCAAGAGAAGAGGCGCCGTACACCAC-3′ | (SEQ ID NO: 42) |
| \|---Sense Strand---\|--Loop--\|-Antisense Strand-\| | |
| 5′-CTGACAGCAACGTCTATGATTCAAGAGATCATAGACGTTGCTGTCAG-3′ | (SEQ ID NO: 43) |
| \|---Sense Strand---\|--Loop--\|-Antisense Strand-\| | |
| 5′-GTATTCATTCCTGCAGAACTTCAAGAGAGTTCTGCAGGAATGAATAC-3′ | (SEQ ID NO: 44) |
| \|---Sense Strand---\|--Loop--\|-Antisense Strand-\| | |
| 5′-CAGCCTGGTGTACGGCGCCTTCAAGAGAGGCGCCGTACACCAGGCTG-3′ | (SEQ ID NO: 45) |
| \|---Sense Strand---\|--Loop--\|-Antisense Strand-\| | |
| 5′-CGATGACGCACTACTCAAGTTCAAGAGACTTGAGTAGTGCGTCATCG-3′ | (SEQ ID NO: 46) |
| \|---Sense Strand---\|--Loop--\|-Antisense Strand-\| | |

In another preferred aspect, the iRNA comprises a nucleotide sequence selected from the group consisting of:

| | |
|---|---|
| 5′-XXXXGAGGGCAGTGCCTTCCCAATTCAAGAGATTGGGAAGGCACTGCCCTCXXXX-3′ | (SEQ ID NO: 47) |
| \|---Sense Strand---\|--Loop--\|-Antisense Strand-\| | |
| 5′-XXXXGCCTATATCCCAAAGGAACTTCAAGAGAGTTCCTTTGGGATATAGGCXXXX-3′ | (SEQ ID NO: 48) |
| \|---Sense Strand---\|--Loop--\|-Antisense Strand-\| | |
| 5′-XXXXGCCTGGTGTACGGCGCCTCTTTCAAGAGAAGAGGCGCCGTACACCAGXXXX-3′ | (SEQ ID NO: 49) |
| \|---Sense Strand---\|--Loop--\|-Antisense Strand-\| | |
| 5′-XXXXGCTGACAGCAACGTCTATGATTCAAGAGATCATAGACGTTGCTCTCAGXXXX-3′ | (SEQ ID NO: 50) |
| \|---Sense Strand---\|--Loop--\|-Antisense Strand-\| | |
| 5′-XXXXGTATTCATTCCTGCAGAACTTCAAGAGAGTTCTGCAGGAATGAATACXXXX-3′ | (SEQ ID NO: 51) |
| \|---Sense Strand---\|--Loop--\|-Antisense Strand-\| | |
| 5′-XXXXGCAGCCTGGTGTACGGCGCCTTCAAGAGAGGCGCCGTACACCAGCCTGXXXX-3′ | (SEQ ID NO: 52) |
| \|---Sense Strand---\|--Loop--\|-Antisense Strand-\| | |
| 5′-XXXXGCGATGACGCACTACTCAAGTTCAAGAGACTTGAGTAGTGCGTCATCGXXXX-3′ | (SEQ ID NO: 53) |
| \|---Sense Strand---\|--Loop--\|-Antisense Strand-\| | |

“X” indicates any number of additional nucleotides which may be present; for example termination signals and restriction sites which may be of use in cloning and expressing the iRNA.

By way of non-limiting examples, the following nucleic acids may be used to clone and express, in desired vectors, the iRNAs of use in the invention:

| | |
|---|---|
| BamHI Hind III | |
| 5′-ggatccGAGGGCAGTGCCTTCCCAATTCAAGAGATTGGGAAGGCACTGCCCTCTTTTTTGGAAaagtcc-3′ | (SEQ ID NO: 54) |
| \| Sense Strand \| Loop \| Antisense Strand \|Terminator | |
| BamHI Hind III | |
| 5′-ggatccGCCTATATCCCAAAGGAACTTCAAGAGAGTTCCTTTGGGATATAGGCTTTTTTGGAAaagtcc-3′ | (SEQ ID NO: 55) |
| \| Sense Strand \| Loop \| Antisense Strand \|Terminator | |
| BamHI Hind III | |
| 5′-ggatccGCCTGGTGTACGGCGCCTCTTTCAAGAGAAGAGGCGCCGTACACCAGGCTTTTTTGGAAaagtcc-3′ | (SEQ ID NO: 56) |
| \| Sense Strand \| Loop \| Antisense Strand \|Terminator | |
| BamHI Hind III | |
| 5′-ggatccGCTGACAGCAACGTCTATGATTCAAGAGTCATAGACGTTGCTGTCAGAGTTTTTTGGAAaagtcc-3′ | (SEQ ID NO: 57) |
| \| Sense Strand \| Loop \| Antisense Strand \|Terminator | |
| BamHI Hind III | |
| 5′-ggatccGTATTCATTCCTGCAGAACTTCAAGAGAGTTCTGCAGGAATGAATACTTTTTTGGAAaagtcc-3′ | (SEQ ID NO: 58) |
| \| Sense Strand \| Loop \| Antisense Strand \|Terminator | |
| BamHI Hind III | |
| 5′-ggatccGCAGCCTGGTGTACGGCGCCTTCAAGAGAGGCGCCGTACACCAGGCTGTTTTTTGGAAaagtcc-3′ | (SEQ ID NO: 59) |
| \| Sense Strand \| Loop \| Antisense Strand \|Terminator | |
| BamHI Hind III | |
| 5′-ggatccGCGATGACGCACTACTCAAGTTCAAGAGACTTGAGTAGTGCGTCATCGTTTTTTGGAAaagtcc-3′ | (SEQ ID NO: 60) |
| \| Sense Strand \| Loop \| Antisense Strand \|Terminator | |

iRNA molecules can be produced in accordance with techniques described within the section entitled “Examples” herein. Further information regarding how to produce and design such molecules can be gained, for example, from: McManus M T and Sharp P A (2002) Gene silencing in mammals by small interfering RNAs. Nature Rev. Genet. 3: 737747; Dillin A (2003) The specifics of small interfering RNA specificity. Proc. Natl. Acad. Sci. USA 100(11): 6289-6291; and Tuschl T (2002) Expanding small RNA interference. Nature Biotechnol. 20: 446-448.

It will be understood by one of skill in the art that siRNA refers to short/small interfering RNA, which comprises double-stranded RNA, typically including 21 to 23 base pairs, which can be chemically synthesized. By comparison, shRNA refers to short hairpin RNA, also called vector based siRNA, which comprises single strand RNA, for example, transcribed in vitro or in vivo. Typically, shRNA includes a sequence homologous to the target mRNA (sense sequence), a “loop” region and a sequence complementary to the target sequence (anti-sense sequence). The shRNA forms a hair-pin secondary structure, and an enzyme dicer cleaves the structure, removes the hairpin, and converts it into siRNA. Thus, the sequences disclosed herein can be used to produce shRNAs, and then converted to siRNAs, as desired.

In another embodiment of the invention, an antisense molecule is used. As used herein, the term “antisense” should be taken broadly. It is intended to mean any nucleic acid (preferably RNA, but including single stranded DNA) capable of binding to a hormone transcript. Typically, antisense molecules or oligonucleotides comprise about 15 to 25 nucleotides which are completely complementary to their target mRNA. However, it should be appreciated that larger antisense oligonucleotides can be used including full length sequences. Also, it should be appreciated that antisense molecules which are not completely complementary to their targets may be utilised provided they retain specificity for their target and the ability to inhibit expression.

Persons of ordinary skill in the art to which the invention relates will appreciate antisense molecules of use in the invention having regard to the description provided herein, and available sequence data. Further information regarding antisense technology can be gained, for example, from: Kandimalla E R, Manning A, Lathan C, Byrn R A, Agrawal S. Design, biochemical, biophysical and biological properties of cooperative antisense oligonucleotides; Nucleic Acids Res. 1995 Sep. 11; 23(17):3578-84; Tseng B Y, Brown K D. Antisense oligonucleotide technology in the development of cancer therapeutics; Cancer Gene Ther. 1994 March; 1(1):65-71; Brysch W, Schlingensiepen K H. Design and application of antisense oligonucleotides in cell culture, in vivo, and as therapeutic agents; Cell Mol. Neurobiol. 1994 October; 14(5):557-68; Han J, Zhu Z, Hsu C, Finley W H. Selection of antisense oligonucleotides on the basis of genomic frequency of the target sequence; Antisense Res Dev. 1994 Spring; 4(1):53-65.

It should be appreciated that DNAzymes, single stranded DNA, ribozymes, and triple helix DNA may also be of use in inhibiting a hormone in accordance with the invention. Ribozymes, DNAzymes, triple helix, and single stranded DNA may be readily appreciated by persons of general skill in the art to which the invention relates having regard to the description provided herein, available sequence data and current methodologies. However, by way of example methodology associated with these technologies is described in Joseph Sambrook and David W. Russell. Molecular Cloning: A Laboratory Manual (Third Edition), Cold Spring Harbor Laboratory Press, NY.

Polynucleotides of use in the invention, including antisense, iRNA, ribozymes and DNAzymes may be chemically modified to increase stability or prevent degradation or otherwise. For example, the nucleic acid molecules may include analogs with unnatural bases, modified sugars, especially at the 2′ position of the ribose, or altered phosphate backbones. Polynucleotides of use in the invention may also include sequences which allow for targeted degradation of any transcript to which they bind. For example, a sequence specific for RNase H, may be included. Another example is the use of External Guide Sequences (EGSs), which may recruit a ribozyme (RNase P) to digest the transcript to which an antisense molecule is bound.

One can help ensure specificity of the likes of antisense oligonucleotides, iRNAs, ribozymes, DNAzymes, and cDNAs by screening candidate sequences for homology with other sequences in the transcriptome, the full complement of activated genes, mRNAs, or transcripts in a particular cell. Also, skilled persons may appreciate appropriate algorithms of use in designing and ensuring specificity of such polynucleotides.

Polynucleotides of use in the invention may be used in the form of nucleic acid molecules produced in vitro, for example, single stranded DNA, iRNA, antisense RNA, or DNAzymes. Alternatively, where appropriate, they may be used in the form of a vector adapted to produce appropriate nucleic acids, for example, antisense molecules, iRNA, or ribozymes. The inventors contemplate the use of any vectors as may be known in the art. For example, naked plasmids that employ CMV promoters may be used. Viral vectors may also be suitable, such as adeno-associated virus (AAV) and lentiviruses. Other examples of suitable promoters and viral vectors are provided herein after. One advantage of using such viral vectors is that they may allow for systemic administration, as opposed to localised administration to a tissue or tumour.

The vectors or constructs of use in the invention may include appropriate genetic elements, such as promoters, enhancers, origins of replication as are known in the art, including inducible, constitutive, or tissue-specific promoters. In a specific embodiment, a vector comprises an inducible promoter operably linked to the region coding a nucleic acid of the invention (for example, antisense or suitable siRNA), such that expression of the nucleic acid is controllable by controlling the presence or absence of the appropriate inducer of transcription. In another embodiment, nucleic acid molecules of the invention are flanked by regions that promote homologous recombination at a desired site in the genome, thus providing for intrachromosomal integration of the desired nucleic acids (Koller and Smithies, 1989, Proc. Natl. Acad. Sci. USA 86:8932-8935; Zijlstra et al., 1989, Nature 342:435-438). Of course, the vectors may remain extrachromosomal.

In another embodiment of the invention, PNAs are used. PNAs are peptide-nucleic acid hybrids in which the phosphate backbone has been replaced by an achiral and neutral backbone made from N-(2-aminoethyl)-glycine units (see, e.g., Eurekah Bioscience Collection. PNA and Oligonucleotide Inhibitors of Human Telomerase. G. Gavory and S. Balasubramanian, Landes Bioscience, 2003). The bases A, G, T, C are attached to the amino nitrogen on the backbone via methylenecarbonyl linkages (P. E. Nielsen et al., Science 1991. 254: 1497-1500; M. Egholm et al., Nature 1993. 365: 566-568). PNAs bind complementary sequences with high specificity, and higher affinity relative to analogous DNA or RNA (M. Egholm et al., supra). PNA/DNA or PNA/RNA hybrids also exhibit higher thermal stability compared to the corresponding DNA/DNA or DNA/RNA duplexes (M. Egholm et al., supra). PNAs also possess high chemical and biological stability, due to the unnatural amide backbone that is not recognized by nucleases or proteases (V. Demidov et al., Biochem Pharmacol 1994. 48: 1310-1313). Typically, PNAs are at least 5 bases in length, and include a terminal lysine. PNAs may be pegylated to further extend their lifespan (Nielsen, P. E. et al. (1993) Anticancer Drug Des. 8:53-63). As non-limiting examples, antigene PNAs can be provided as complementary to unique sequences in the coding DNA strand of the gene, and designed to inhibit mRNA synthesis.

Antibodies for Inhibition of GH and/or Related Hormones

The invention encompasses antibodies to GH and/or a related hormone, particularly hGH or hormone encoded by the human growth hormone gene cluster, a proliferin gene product or a prolactin gene product, e.g., hPRL, or hPL. For example, antibodies that bind to at least a portion of the hormone or a modified sequence thereof. In certain aspects of the invention, antibodies may be used to inhibit one or more of these hormones. It will be understood that, for the purposes of the invention, a fragment or modification of an antibody need not act fully as an antibody. That is to say, the fragment or modification need not be capable of recruiting immune system cells to the site of binding to the hormone in vivo. It is not necessary to produce neutralising antibodies. Those of ordinary skill in the art to which the invention relates will recognise methods to generate antibody fragments. Antibody fragments of the invention can encompass a portion of one of the intact antibodies, generally the antigen binding or variable region of the antibody. By way of general example, fragments can be generated proteolytic digestion of intact antibodies may be used, or the fragments may be directly produced via recombinant nucleic acid technology. The inventors believe that the antibodies, antibody fragments, or modifications thereof, as described herein, find application in the regulation of hormone-mediated functions. Particularly, the inventors have discovered that antibodies can be used to inhibit hormone levels and/or activity and therefore may be applicable to the treatment of various disorders in a subject.

It is understood that the basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The antibodies of the invention can be characterised on the basis of isotype and epitope mapping and, in some cases, their ability to block binding of the hormone to its receptor. The inventors have identified domains of each hormone which would be useful to generate blocking antibodies. Reagents that block hormone binding or activation have application in methods for treatment of various disorders as disclosed herein. Antibodies that recognize GH and/or a related hormone, particularly hGH and/or variants thereof, hPRL, or hPL, but fail to block receptor binding are also contemplated. Such antibodies find use in detecting and purifying such hormones, as well as in methods for diagnosing and monitoring progression of the disorders in a subject as detailed herein after. Such antibodies might also find use in analysing the post-translational modifications of the hormone, or other modified sequences thereof.

Humanization of antibodies may be used to reduce the immunogenicity of antibodies generated in other animals. Production of humanised antibodies or humanization of antibodies can be achieved using techniques known in the art. The most frequently used strategies for the humanization of rodent monoclonal antibodies are CDR grafting (Reichmann, L., M. Clark, H. Waldman, and G Winter. 1998. Reshaping human antibodies for therapy. Nature 332L323-327; Jones P T, Dear P H, Foote J, Neuberger M S, Winter G 1986. Replacing the complementarity determining regions in a human antibody with those from a mouse. Nature 321:522-25) and resurfacing (Pedersen, J. T., A. H. Henry, S. J. Searle, B. C. Guild, M. Roguska, and A. R. Rees. 1994. Comparison of surface accessible residues in human and murine immunoglobulin Fv domains. Implication for humanization of murine antibodies. J. Mol. Biol. 235:959-973). Humanization of antibodies can also be achieved by epitope-guided selection (Wang et al, J. Immunological Methods 241: 171-184, 2000). The methods of Maynard, J., and Georgiou, G 2000. Antibody engineering. Annu Rev Biomed Eng 2: 339-376, provide further examples.

Humanized antibodies can be produced based on a rational design approach and iterative optimization, i.e., site-directed mutagenesis of framework residues aided by computer modelling. Other selective humanization strategies using phage display can be used. See, e.g., Baca, M., L. G. Presta, S. J. O'Connor, and J. A. Wells. 1997. Antibody humanization using monovalent phage display. J. Biol. Chem. 272:10678-10684; Hoogenboom, H. R., A. P. de Bruine, S. E. Hufton, R. M. Hoet, J. W. Arends, and R. C. Roovers. 1998. Antibody phage display technology and its applications. Immunotechnology. 4:1-20; Jespers, L. S., A. Roberts, S. M. Mahler, G. Winter, and H. R. Hoogenboom. 1994. Guiding the selection of human antibodies from phage display repertoires to a single epitope of an antigen. Biotechnology (NY) 12:899-903; Rader, C. and C. F. Barbas, III. 1997. Phage display of combinatorial antibody libraries. Curr. Opin. Biotechnol. 8:503-508; Rader, C., D. A. Cheresh, and C. F. Barbas, III. 1998. A phage display approach for rapid antibody humanization: designed combinatorial V gene libraries. Proc. Natl. Acad. Sci. U.S. A 95:8910-8915; Rosok, M. J., D. E. Yelton, L. J. Harris, J. Bajorath, K. E. Hellstrom, I. Hellstrom, G. A. Cruz, K. Kristensson, H. Lin, W. D. Huse, and S. M. Glaser. 1996. A combinatorial library strategy for the rapid humanization of anticarcinoma BR96 Fab. J. Biol. Chem. 271:22611-22618.

In other aspects, human antibodies can be produced using a transgenic animal strain reconstituted with human immunoglobulin loci, e.g., XenoMouse strains. Such strains make it possible to generate fully human antibodies in an animal host (Mendez, M. J., L. L. et al. 1997. Functional transplant of megabase human immunoglobulin loci recapitulates human antibody response in mice. Nat. Gen. 15:146-156). XenoMouse animals, in particular, comprise yeast artificial chromosomes (YACs) containing 66 human heavy-chain and 32 kappa light-chain immunoglobulin genes in their genome, where endogenous heavy-chain and kappa loci are functionally inactivated by targeted deletion (Mendez et al., supra). The mice express human mu, delta, gamma 2, and kappa chains and mouse lambda chains, with a human kappa-to-mouse lambda ratio of 75:1 (Mendez et al., supra). XenoMouse animals have been previously shown to produce human antibodies to protein antigens (Green, L. L., et al. 1994. Antigen-specific human monoclonal antibodies from mice engineered with human Ig heavy and light chain YACs. Nat. Gen. 7:13-21; Mendez et al., supra), and may also respond to T-independent antigens such as polysaccharides. As one example, two or more genetically distinct groups of XenoMouse animals can used to produce antibodies, for example, Xm2a-3 strains, reconstituted with one double YAC containing both heavy- and light-chain genes; and Xm2a-5 strains, reconstituted with two YACs, one with heavy-chain and the other with light-chain genes (Jakobovits, A. 1995. Production of fully human antibodies by transgenic mice. Curr. Opin. Biotechnol. 6:561-566; see, also, Russell N D et al., Production of protective human antipneumococcal antibodies by transgenic mice with human immunoglobulin loci. Infect Immun. 2000 April; 68(4):1820-6).

Those of skill in the art to which the invention relates will appreciate the terms “diabodies” and “triabodies”. These are molecules which comprise a heavy chain variable domain (VH) connected to a light chain variable domain (VL) by a short peptide linker that is too short to allow pairing between the two domains on the same chain. This promotes pairing with the complementary domains of one or more other chains and encourages the formation of dimeric or trimeric molecules with two or more functional antigen binding sites. The resulting antibody molecules may be monospecific or multispecific (e.g., bispecific in the case of diabodies). Such antibody molecules may be created from two or more antibodies using methodology standard in the art to which the invention relates. See, e.g., Todorovska et al. Design and application of diabodies, triabodies and tetrabodies for cancer targeting. J. Immunol. Methods. 2001 Feb. 1; 248(1-2):47-66; Holliger P, Prospero T, and Winter G, Diabodies: small bivalent and bispecific antibody fragments, Proc Natl Acad Sci USA, 90, 6444-6448, 1993; and Tomlinson I and Holliger P, Methods for generating multivalent and bispecific antibody fragments, Methods Enzymol, 326, 461-479, 2000.

The production of antibodies may be carried out according to standard methodology in the art. For example, in the case of the production of polyclonal antibodies the methodology described by Bean (Eric S. Bean (2001) Polyclonal Antibodies. In: Basic Methods in Antibody Production and Characterization antibodies. Howard, G, and Bethel D. (ed.), CRC Press, 5:21-50, 2000) may be used. Monoclonal antibodies and corresponding hybridomas may be prepared, for example, in accordance with the methodology of Stewart (Sandy J. Stewart (2001) Monoclonal Antibody Production. In: Basic Methods in Antibody Production and Characterization antibodies. Howard, G. and Bethel D. (ed.), CRC Press, 6:51-68, 2000) or in Monocolonal Antibody Production Techniques and Applications, Lawrence B Schook eds., Marcel Dekker Inc., New York, 1987. Hybridomas may be subcloned, grown and maintained using standard techniques in the art. For example, they may be grown and maintained in vitro in media such as DMEM or RPMI-1640. Alternatively, this may be done in vivo as ascites tumours in an animal of choice.

The antibodies of the invention may be isolated for example, from culture supernatants, ascites fluid, or serum using standard procedures known in the art to which the invention relates. An example of such techniques is provided herein below. However, as other examples, isolation or purification may occur via one or more of procedures such as affinity chromatography, ion exchange chromatography, interaction chromatography, gel filtration chromatography, thiophilic gel chromatography, chromatofocusing, Protein-A or G. Sepharose columns, hydroxyapatite columns, detergent extraction, electrophoresis, osmotic shock treatment, inclusion body purification, ammonium sulphate precipitation, centrifugation with liquid polymers, filtration, and dialysis. A recombinant antibody in accordance with the invention may be recovered from a transformed host cell, or culture media, or transgenic organism using a variety of techniques that are standard in the art. It will be appreciated that the amino acid sequence of an antibody of the invention may be determined using standard methodology, for example, using Edman degradation and HPLC, or mass spectroscopy analysis (M. W. Hunkapiller, R. M. Hewick, W. J. Dreyer, and L. E. Hood. 1983. High-Sequencing with a Gas-Phase Sequenator. Methods Enzymol. 91: 399).

Standard methods can be used to identify amino acid sequences useful for antibody production. Antigenic segments of a polypeptide can be predicted, for example, by Abie Pro 3.0: Peptide Antibody Design (hypertext transfer protocol://world wide web.changbioscience.com/abie/abie.html). More particularly, antigenic segments can be predicted according to the Hopp-Woods scale (Hopp T P, Woods K R. Prediction of protein antigenic determinants from amino acid sequences. Proc Natl Acad Sci USA. 1981 June; 78(6):3824-8.) and/or the Kyte and Doolittle scale (Kyte J, Doolittle R F. A simple method for displaying the hydropathic character of a protein. J Mol. Biol. 1982 May 5; 157(1):105-32.). Particular computer programs include MAPAG (Aguilar R C, Retegui L A, Roguin L P Int J Biomed Comput. 1994 November-December;37(3):225-35); PEOPLE (Alix A J.: Vaccine. 1999 September; 18(3-4):311-4); and HYDRPHIL (E A Mesri et al. J Clin Microbiol. 1990 June; 28(6): 1219-1224).

Such epitopes may be conformational specific, in that they may include non-contiguous residues, and may constitute various portions of the predicted antigenic sequences (e.g., portions of any one of SEQ ID NO: 10-20, see below), or a combination of one or more portions of the predicted antigenic sequences (e.g., combinations of one or more of SEQ ID NO: 10-20), or one or more of the full length sequences (e.g., one or more of SEQ ID NO: 10-20). Antigenic sequences can comprise any combination of amino acids or their derivatives that would form a similar 3-D structure (e.g., surface residues) as would be encountered in the native polypeptide.

Table 1, below, shows predicted antigenic determinants (ADs) for hGH. Table 2 shows predicted conformational epitopes for hGH. Table 3 shows predicted sequential epitopes for hGH. Table 4 shows predicted antigenic determinants for hPRL. Table 5 shows predicted conformational epitopes for hPRL. In these tables, lower case letters indicate amino acids less likely to be involved in generating the 3D conformation of the peptide (i.e., less likely to serve as antigenic determinants), whereas capital letters indicate amino acids more likely to be involved in generating the 3D conformation of the peptide (i.e., more likely serve as antigenic determinants).

TABLE 1

| AD | | SEQ |
|---|---|---|
| No. | Predicted AD | ID NO: |
| 1 | X_2: PTIPLSRLfQN: 12 | 10 |
| 2 | X_33: EAY: 35 | 11 |
| 3 | X_40: QKYSFIQALPQASL: 52 | 12 |
| 4 | X_56: ESiPTpSNREQaQQKSNIQ: 74 | 13 |
| 5 | X_91: GFLRSVfANSLVYGASDSDVYD: 112 | 14 |
| 6 | X_126: GRLEDGSPRTGqAFKQTYAkFDaNSHNDD: 154 | 15 |
| 7 | X_182: CRSVEGSCG: 190 | 16 |

TABLE 2

| | | | |
|---|---|---|---|
| | | Residues | |
| | | within 6Å | SEQ |
| CE No. | Predicted CE | of Ref AD | ID NO: |

| | | | |
|---|---|---|---|
| 1 | X_2: PTIPLSRLfQN: 12 | X_15: L | 10 |
| | X_182: GRSVEGSCG: 190 | X_120: G | 16 |
| 2 | X_56: ESiPTpSNREQaQQKSNIQ: 74 | X_77: R | 13 |
| | X_126: GRLEDGSPRTGqAFKQTYAkFDaNSHNDD: 154 | X_78: I | 17 |
| | X_182: CRSVEGSCG: 190 | | 16 |
| 3 | X_91: GFLRSVfANSLVYGASDSDVYD: 112 | X_19: R | 14 |
| | X_126: GRLEDGSPRTGqAFKQTYAkFDaNSHNDD: 154 | X_88: E | 15 |
| | | X_115: K | |
| | | X_116: D | |
| 4 | X_126: GRLEDGSPRTGqAFKQTYAkFDaNSHNDD: 154 | X_77: R | 15 |
| | X_91: GFLRSVfANSLVYGASDSDVYD: 112 | X_84: Q | 14 |
| | X_56: ESiPTpSNREQaQQKSNIQ: 74 | X_888: E | 13 |
| | | X_122: Q | |
| | | X_123: T | |
| | | X_157: L | |
| 5 | X_182: CRSVEGSCG: 190 | X_178: R | 16 |
| | X_2: PTIPLSRLfQN: 12 | | 10 |
| | X_56: ESiPTpSNREQaQQKSNIQ: 74 | | 13 |

TABLE 3

| | | | |
|---|---|---|---|
| | | Residues | |
| | | within 6Å | SEQ |
| SE No. | Predicted SE within 6Å of Reference AD | of Ref AD | ID NO: |

| | | | |
|---|---|---|---|
| 1 | X_40: QKYSFIQAPQASL: 52 | X_157: L | 12 |
| 2 | X_33: EAY: 35 | X_29: E | 11 |
| 3 | X_37: PKE: 39 | | 17 |
| | X_33: EAYIPKE: 39 | | 18 |
| | X_37: PKEQKYSFIQAPQASL: 52 | | 19 |
| | X_33: EAYIPKEQKYSFIQAPQASL: 52 | | 20 |

It should be noted that the protein databank for hGH NMR coordinates (pdb file id: 1HGU) had missing information for amino acids 37, 38, and 39. As these amino acids fall outside the core helices, and directly between two SEs, they can include antigenic determinant X_{—}37: PKE: 39, (SEQ ID NO: 17) or may be part of any of the two SEs X_{—}33: EAYIPKE:39 (SEQ ID NO: 18) and X_{—}37: PKEQKYSFIQAPQASL:52 (SEQ ID NO: 19), or may together with the other two SEs form a larger SE, e.g., X_{—}33: EAYIPKEQKYSFIQAPQASL:52 (SEQ ID NO: 20) (see, 3, in the table above).

TABLE 4

| | | SEQ |
|---|---|---|
| AD | Predicted AD | ID NO: |
| 1_A | A_1-LPICPGGaArCQV-13 | 21 |
| 2_A | A_44-YtHGRgFiTkAiNScHtSSLATPEDKEQ-71 | 22 |
| 3_A | A_74-QMNQ-77 | 23 |
| 4_A | A_103-RGmQeAPEAiLS-114 | 24 |
| 5_A | A_135-SQvHpETKENEiYPVWSGLPSIQmADEEsR-164 | 25 |
| 6_A | A_187-K1LKcRIIhNNNC-199 | 26 |

TABLE 5

| | | Res. within 6 | SEQ |
|---|---|---|---|
| CE No. | CE within 6Å of Reference AD | Å of Ref. AD | ID NO: |
| 1 | A_1: LPICPGGaArCQV: 13 | | 21 |
| | A_135: SQvHpETKENEiYPVWSGLPS1QmADEEsR: 164 | A_16: R | 25 |
| | A_187: K1LKcRIIhNNNC: 199 | A_20: D | 26 |
| 2 | A_44: YtHGRgfiTkAiNScHtSSLATPEDKEQ: 71 | A_37: F | 22 |
| | A_74: QMNQ: 77 | A_38: S | 23 |
| | A_103: RGmQeAPEAiLS: 114 | A_170: N | 24 |
| | A_135: SQvHpETKENEiYPVWSGLPSIQmADEEsR: 164 | A_177: R | 25 |
| | A_187: K1LKcRIIhNNNC: 199 | A_180: H | 26 |
| 3 | A_74: QMNQ: 77 | | 23 |
| | A_44: YtHGRgFiTkAiNScHtSSLATPEDKEQ: 71 | | 22 |
| | A_135: SQvHpETKENEiYPVWSGLPS1QmADEEsR: 164 | | 25 |
| | A_187: K1LKcRIIhNNNC: 199 | | 26 |
| 4 | A_103: RGmQeAPEAiLS: 114 | A_31: N | 24 |
| | A_44: YtHGRgFiTkAiNScHtSSLATPEDKEQ: 71 | A_35: E | 22 |
| | A_135: SQvHpETKENEiYPVWSGLPS1QmADEEsR: 164 | A_38: S | 25 |
| | | A_100: T | |
| | | A_117: V | |
| | | A_118: E | |
| 5 | A_135: SQvHpETKENEiYPVWSGLPS1QmADEEsR: 164 | | 25 |
| | A_1: LPICPGGaArCQV: 13 | A_89: R | 21 |
| | A_44: YtHGRgFiTkAiNScHtSSLATPEDKEQ: 71 | A_90: S | 22 |
| | A_74: QMNQ: 77 | A_93: E | 23 |
| | A_103: RGmQeAPEAiLS: 114 | A_97: H | 24 |
| 6 | A_187: K1LKcRIIhNNNC: 199 | | 26 |
| | A_1: LPICPGGaArCQV: 13 | | 21 |
| | A_44: YtHGRgFiTkAiNScHtSSLATPEDKEQ: 71 | A_16: R | 22 |
| | A_74: QMNQ: 77 | | 24 |

Antibodies of use in the invention may also be produced via standard recombinant techniques, see, e.g., Siegel (2002) Siegel D L, Recombinant monoclonal antibody technology, Transfus Clin Biol, 9(1):15-22 2002; Welschof, M., C. Christ, I. Hermes, A. Keller, C. Kleist, and M. Braunagel. 2003. Generation and screening of a modular human scFv expression library from multiple donors. Methods Mol. Biol. 207:103-121). The inventors consider recombinant techniques to be a preferable means of producing antibodies on a commercial scale. Polynucleotides encoding an antibody may be readily identified on the basis of the amino acid sequence of the antibody, the genetic code, and the understood degeneracy therein. Polynucleotides encoding antibodies may be isolated from hybridoma cells, for example, and subsequently characterised using procedures standard in the art. For example, a polynucleotide probe may be designed based on the amino acid sequence of a portion of an antibody and then used to isolate genes encoding the heavy and/or light chains of the antibody. Alternatively, polynucleotides may be generated by standard chemical synthesis methodology, for example, using phosphoramidite and solid phase chemistry.

It should be appreciated that inasmuch as the invention also extends to fragments, as well as modifications of the antibodies specifically referred to herein, nucleic acid encoding the antibodies may be appropriately modified. For example, the coding sequence for heavy- and light-chain constant domains may be replaced with an homologous human domain. Alternatively, the CDR (complementarity determining region or antigen binding site) regions may be transplanted to a homologous human beta-sheet framework. In this way, antibody modifications, such as humanised antibodies may be generated via recombinant techniques. As mentioned herein, antibodies of the invention may be produced via standard recombinant procedures. Accordingly, the present invention also extends to polynucleotides encoding an antibody, antibody fragment, or modification thereof, constructs comprising same, and host cells comprising said constructs. Polynucleotides in accordance with the invention may be DNA, RNA, or cDNA, for example, double stranded or single stranded, sense or antisense sequences, as described herein.

As will be appreciated, the antibodies, or antibody fragments, or modifications thereof in accordance with the invention may be used for the general purposes of detection and purification of a hormone. The hormone may be from a natural or artificial source, such as a cell culture. Preferably, the hormone is of human origin. Additionally, as may be useful for certain applications, antibodies may be modified by labelling with a compound which provides a detectable signal. For example, enzymes, fluorescent agents, and radioisotopes can be used. Those of general skill in the art to which the invention relates will readily identify such suitable labelling systems. Thus, in addition to therapeutic use of antibodies directed against hormones, the antibodies may find use in purification of the hormones or in diagnostic applications. For example, antibodies immobilised on a solid phase would aid in purification and/or quantitation of the level of hormone in a sample. Those of ordinary skill in the art to which the invention relates will appreciate techniques by which this may be done. However, by way of example, affinity chromatography using antibodies, antibody fragments, or modifications may be used immobilised on a chromatographic support. In the case of diagnostic and purification procedures, it is not necessary for the antibody to have inhibitory activity.

It will be appreciated that ELISA or similar assays may incorporate both direct and indirect detection means, and that an antibody of the invention, or antibody fragment, or modification thereof, may be used as either capture or detection antibodies. As will be appreciated, one or more of the antibodies of the invention may be used in a single assay. For example, where two antibodies of the invention do not recognise the same antigenic determinant on a hormone, one antibody may be used as a capture antibody and the other antibody may be used as a detection antibody. Alternatively, the antibodies of the invention can be used in combination with previously identified antibodies to the hormones. As will be appreciated by persons of ordinary skill in the art, the detection antibody used in an ELISA may be conjugated to a detectable label as herein described.

Information of use in diagnosing or generally monitoring the status of a subject may be gained by making a direct comparison of the level of hormone in a test sample, with that of a determined base level or standard. For example, the average serum level of GH and/or a related hormone, in particular, hGH or variants thereof, hPRL, or hPL, for a normal subject can be determined (i.e., a subject known not to present a medical disorder as described herein). These concentrations may be used as base levels, with a result above this range being indicative of a medical disorder. Preferably, the level necessary to be indicative of a disorder is a statistically significant increase of those ranges identified as normal. However, even where there is no statistically significant increase, results obtained may provide valuable information about the status of a subject. It should be appreciated that the normal ranges of hormone may differ in different body fluids and tissues. Similarly, normal levels of localised hormone may fall outside the range for normal levels in serum.

It should be appreciated that diagnosis or general determination of a subject's status may be made by comparing the level of hormone present in a test sample against a database of results obtained from a range of other subjects. Instead of utilising a standard or base level concentration of hormone obtained from a number of normal subjects, the base level concentration may be determined from a single subject during a period when they were known not to present a medical disorder, or during a period of an active medical disorder. This may be particularly applicable to cases of ongoing and/or intermittent disease events or disorders where constant monitoring of the subjects status is required. For example, a base level may be determined during a period of remission from the disorder and the diagnostic procedure carried out at various times thereafter to assess status. This may provide valuable information pertaining to progression of a disorder, or help in assessing whether treatment of the disorder is proving successful.

As described herein, antibodies produced in accordance with the invention may find particular use as therapeutic agents, for example, for preventing, decreasing, or inhibiting cell proliferation, cell survival, or cell motility. In one broad embodiment the invention provides a method of blocking the interaction of at least one hormone with one or more hormone receptors, or more broadly, blocking the interaction of a hormone with a binding agent, the method comprising contacting the antibody, antibody fragment, or modification thereof in accordance with the invention. This method may be conducted in vivo or in vitro. Persons of ordinary skill in the art will readily appreciate methods for determining the efficacy of an antibody in preventing, decreasing, or inhibiting cell proliferation, cell survival, or cell motility. However, by way of example, the methodology described elsewhere herein, including one or more of the assays referred to in the “Examples” section, may be used. Additionally, the antibodies of the invention may be used as carriers, for example to carry toxins, radionucleotides, isotopes, genes, or other therapeutic molecules to cells or tissues to aid in therapy.

Compositions for Inhibition of GH and/or Related Hormones

The agents of use in inhibiting GH and/or a related hormone, particularly hGH and/or variants thereof, hPRL, or hPL, may be used on their own, or in the form of compositions in combination with one or more pharmaceutically acceptable diluents, carriers, and/or excipients. Those skilled in the art to which the invention relates will readily appreciate a variety of pharmaceutically acceptable diluents, carriers, and/or excipients which may be employed in compositions of the invention. As will be appreciated, the choice of such diluents, carriers, and/or excipients will be dictated to some extent by the nature of the agent to be used, the intended dosage form of the composition, and the mode of administration. By way of example, in the case of administration of polynucleotides, such as vectors adapted to express antisense or iRNA, suitable carriers include isotonic solutions, water, aqueous saline solution, aqueous dextrose solution, and the like.

In addition to standard diluents, carriers, and/or excipients, a pharmaceutical composition of the invention may be formulated with additional constituents, or in such a manner, so as to enhance the activity of the agent or help protect the integrity of the agent. For example, the composition may further comprise adjuvants or constituents which provide protection against degradation, or decrease antigenicity of an agent, upon administration to a subject. Alternatively, the agent may be modified so as to allow for targeting to specific cells, tissues, or tumours.

Agents may be formulated to incorporate a sustained-release system. Inasmuch as this is the case, compositions may include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate (Sidman et al., 1983, Biopolymers: 22: 547-56), poly(2-hydroxyethyl methacrylate) (Langer et al., 1981, J. Biomed. Mater. Res.: 15: 267), ethylene vinyl acetate (Langer et al., 1981, J. Biomed. Mater. Res.: 15: 267), or poly-D-(−)-3-hydroxybutyric acid (EP 133,988).

Agents of the invention may also be formulated into liposomes. Liposomes comprising the compound may be prepared using techniques known in the art to which the invention relates. By way of example see: DE 3,218,121, EP 52,322, EP 36,676, EP 88,046, EP 143,949, EP 142,641, Japanese Pat. Appln. 83-118008, U.S. Pat. Nos. 4,485,045 and 4,544,545, and EP 102,324. Ordinarily, the liposomes are of the small (from or about 200 to 800 Angstroms) unilamellar type in which the lipid content is greater than about 30 mol percent cholesterol, the selected proportion being adjusted for the most efficacious therapy. Agents of use in the invention may also be pegylated to increase their lifetime.

Additionally, it is contemplated that a composition in accordance with the invention may be formulated with other ingredients which may be of benefit to a subject in particular instances. For example, there may be benefit in incorporating, where appropriate, one or more anti-neoplastic agents. Examples of such agents include: alkylating agents, for example, chlorambucil (e.g., Leukeran™), cyclophosphamide (e.g., Endoxan™, Cycloblastin™, Neosar™, Cyclophosphamide™), ifosfamide (e.g., Holoxan™, Ifex™, Mesnex™), thiotepa (e.g., Thioplex™, Thiotepa™); and antimetabolites/S-phase inhibitors, for example, methotrexate sodium (e.g., Folex™, Abitrexate™, Edertrexate™), 5-fluorouracil (e.g., Efudix™, Efudex™), hydroxyurea (e.g., Droxia™, Hydroxyurea, Hydrea™), amsacrine, gemcitabine (e.g., Gemzar™), dacarbazine, thioguanine (e.g., Lanvis™).

Also included are antimetabolites/mitotic poisons, for example, etoposide (Etopophos™, Etoposide, Toposar™), vinblastine (e.g., Velbe™, Velban™), vindestine (e.g., Eldesine™), vinorelbine (e.g., Navelbine™), paclitaxel (e.g., Taxol™); antibiotic-type agents, for example, doxorubicin (e.g., Rubex™), bleomycin (e.g., Blenoxane™), dactinomycin (e.g., Cosmegen™), daunorubicin (e.g., Cerubidin™), mitomycin (e.g., Mutamycin™); hormonal agents, for example, aminoglutethimide (e.g., Cytadren™), anastrozole (e.g., Arimidex™), estramustine (e.g., Estracyt™, Emcyt™), goserelin (e.g., Zoladex™), hexamethylmelanine (e.g., Hexamet™), letrozole (e.g., Femara™), anastrozole (e.g., Arimidex™), and tamoxifen (e.g., Estroxyn™, Genox™, Novaldex™, Soltamox™, Tamofen™).

Further included are combinations of any two or more anti-neoplastic agents (for example, Adriamycin/5-fluorouracil/cyclophosphamide (FAC); and cyclophosphamide/methotrexate/5-fluorouracil (CMF)). Particularly useful are combinations that include, for example, at least two or more agents such as cyclophosphamide (e.g., CYTOXAN), methotrexate (e.g., RHEUMATREX), 5-fluorouracil (e.g., ADRUCIL), doxorubicin (e.g., ADRIAMYCIN), and cyclophosphamide (e.g., CYTOXAN). Useful for metastatic disease are agents such as capecitabine (e.g., XELODA), doxorubicin (e.g., ADRIAMYCIN), including its liposomal formulation, gemcitabine (e.g., GEMZAR), the taxanes, including paclitaxel (e.g., TAXOL) and docetaxel (e.g., TAXOTERE), vinorelbine (e.g., NAVELBINE), and trastuzumab (e.g., HERCEPTIN). Persons of ordinary skill in the art to which the invention relates will readily appreciate examples of other agents which may be of benefit. Agents of the invention may also be formulated with compounds and agents, other than those specifically mentioned herein, in accordance with accepted pharmaceutical practice.

As will be appreciated, in the case of administration of polynucleotides, they may be packaged into viral delivery systems, which viral systems may themselves be formulated into compositions as herein described. Persons of skill in the art to which the invention relates may appreciate a variety of suitable viral vectors having regard to the nature of the invention described herein. However, by way of example, retroviral vectors, adenoviral vectors, and adeno-associated virus (AAV) can be used. Persons of skill in the art to which the invention relates will readily appreciate methods which may be employed to implement such vectors in the present invention. However, by way of example only: the use of retroviral vectors is reported in Miller et al., 1993, Meth. Enzymol. 217:581-599, and Boesen et al., 1994, Biotherapy 6:291-302; the use of adenoviral vectors is reported for example in Kozarsky and Wilson, 1993, Current Opinion in Genetics and Development 3:499-503; Rosenfeld et al., 1991, Science 252:431-434; Rosenfeld et al., 1992, Cell 68:143-155; Mastrangeli et al., 1993, J. Clin. Invest. 91:225-234; PCT Publication WO 94/12649; and Wang, et al., 1995, Gene Therapy 2:775-783; and, the use of AAV has been reported in Walsh et al., 1993, Proc. Soc. Exp. Biol. Med. 204:289-300; U.S. Pat. No. 5,436,146.

In accordance with the mode of administration to be used, and the suitable pharmaceutical excipients, diluents and/or carriers employed, compositions of the invention may be adapted into customary dosage forms such as solutions, orally administrable liquids, injectable liquids, tablets, coated tablets, capsules, pills, granules, suppositories, transdermal patches, suspensions, emulsions, sustained release formulations, gels, aerosols, liposomes, powders and immunoliposomes. The dosage form chosen will reflect the mode of administration desired to be used, the disorder to be treated, and the nature of the agent to be used. Particularly preferred dosage forms include orally administrable tablets, gels, pills, capsules, semisolids, powders, sustained release formulations, suspensions, elixirs, aerosols, ointments, or solutions for topical administration, and injectable liquids. Skilled persons will readily recognise appropriate dosage forms and formulation methods. Generally, compositions are prepared by contacting or mixing specific agents and ingredients with one another. Then, if necessary, the product is shaped into the desired formulation. By way of example, certain methods of formulating compositions may be found in references such as Gennaro A R: Remington: The Science and Practice of Pharmacy, 20th ed., Lippincott, Williams & Wilkins, 2000.

The amount of an agent of the invention in a composition can vary widely depending on the type of composition, size of a unit dosage, kind of carriers, diluents and/or excipients, and other factors well known to those of ordinary skill in the art. In general, the final composition can comprise from 0.0001 percent by weight (% w) to 100% w of the actives of this invention, preferably 0.001% w to 10% w, with the remainder being any other active agents present and/or carrier(s), diluent(s) and/or excipient(s).

### Methods of Treatment

While the inventors' primary studies have involved endometrial cancer cells, GH and related hormones, particularly hGH or variants thereof, hPRL, and hPL, are predicted to also act in the small intestine, spleen, liver, fetal liver and kidney, and also heart, prostate, uterus, colon, stomach, skin, lung, trachea, brain, cerebellum, fetal brain, spinal cord, placenta, adrenal gland, adipose, cartilage, hematopoietic and immune systems, pancreas, and also skeletal muscle, thymus, salivary gland, thyroid, umbilical cord, and ovaries Specifically, the hormones are predicted to act in breast cancer, colon cancer, lung cancer, prostate cancer, endometrial cancer, and endometriosis, among other conditions. Accordingly, the inventors contemplate the inhibition of one or more of these hormones being applicable to the treatment of a variety of disorders characterised by altered cell proliferation, cell survival, or cell motility.

In one embodiment, the invention relates to a method of preventing, reducing, or inhibiting cell proliferation, cell survival, or cell motility by inhibiting GH and/or a related hormone, particularly hGH and/or variants thereof, hPRL, or hPL. Preferably, the method is for the treatment of a disorder characterised by aberrant cell in subject. This aberrant cell proliferation, cell survival, or cell motility may occur in one or more cell type within a subject and can include metastatic disorders. Specific disorders include, for example, cancer (breast cancer, colon cancer, lung cancer, prostate cancer, or endometrial cancer, for example) and endometriosis. Examples of disorders include cancers such as adenocarcinoma, leukemia, lymphoma, melanoma, myeloma, sarcoma, teratocarcinoma, and cancers of the adrenal gland, bladder, bone, brain, breast, cervix, gall bladder, ganglia, gastrointestinal tract, heart, kidney, liver, lung, bone marrow, muscle, ovary, pancreas, parathyroid, penis, prostate, salivary glands, skin, spleen, testis, thymus, thyroid, and uterus. In various embodiments, the disorder is an epithelial tumor of the breast, colon, lung, prostate, pancreas, stomach, endometrium, or ovary, or squamous cell carcinoma, or a melanoma, or a renal cancer or tumour.

Regarding breast cancers, these can include epithelial tumours (e.g., from cells lining ducts or lobules) or nonepithelial tumours (e.g., from the supporting stroma), such as angiosarcomas, primary stromal sarcomas, and phyllodes tumor. Breast cancers can also include carcinomas, for example, carcinomas in situ, as well as invasive cancers. Carcinoma in situ includes proliferation of cancer cells within ducts or lobules and without invasion of stromal tissue. Lobular carcinoma in situ (LCIS) includes nonpalpable lesions which can indicate increased risk of subsequent invasive carcinoma in either breast. In breast cancer, invasive carcinoma generally comprises adenocarcinoma, with most comprising infiltrating ductal type carcinoma and the remainder comprising infiltrating lobular carcinoma. Rare forms of breast cancer include medullary, mucinous, and tubular carcinomas. Breast cancer disorders also include Paget's disease of the nipple, and metastatic breast cancer.

Regarding colon cancer, this can generally include cancer of the colon, rectum, and/or anus, and especially, adenocarcinomas, and also carcinomas (e.g., squamous cloacogenic carcinomas), melanomas, lymphomas, and sarcomas. Epidermoid (nonkeratinizing squamous cell or basaloid) carcinomas are also included. The colon cancer may be associated with particular types of polyps or other lesions, for example, tubular adenomas, tubulovillous adenomas (e.g., villoglandular polyps), villous (e.g., papillary) adenomas (with or without adenocarcinoma), hyperplastic polyps, hamartomas, juvenile polyps, polypoid carcinomas, pseudopolyps, lipomas, or leiomyomas. The cancer may be associated with familial polyposis and related conditions such as Gardner's syndrome or Peutz-Jeghers syndrome. The cancer may be associated, for example, with chronic fistulas, irradiated anal skin, leukoplakia, lymphogranuloma venereum, Bowen's disease (intraepithelial carcinoma), condyloma acuminatum, or human papillomavirus. In other aspects, the cancer may be associated with basal cell carcinoma, extramammary Paget's disease, cloacogenic carcinoma, or malignant melanoma.

Regarding endometrial cancers, these can include adenocarcinomas and also papillary serous, clear cell, squamous, and mucinous carcinoma. Also included are precancerous conditions such as endometrial hyperplasia. The endometrial cancer may be associated with one or more of obesity, polycystic ovarian syndrome, nulliparity, late menopause, estrogen-producing tumours, anovulation (ovulatory dysfunction), and estrogen therapy without progesterone and hereditary nonpolyposis colorectal cancer (HNPCC) syndrome.

Persons of general skill in the art to which the invention relates may readily appreciate alternative types of disorder which the invention may be applicable, especially having regard to the expression of the hormone provided herein. In addition, it will be appreciated by those of general skill in the art to which the invention relates, having regard to the nature of the invention and the results reported herein, that the present invention is applicable to a variety of different subjects. Accordingly, the diagnostics and treatments can apply to any subject of interest. In particular, the invention is applicable to mammals, more particularly humans.

Person's of ordinary skill in the art to which the invention relates will appreciate various means and agents of use to inhibit GH and/or a related hormone, particularly hGH and/or variants thereof, hPRL, or hPL. By way of example, nucleic acid technology including iRNA, antisense, and triple helix DNA may be employed to inhibit expression. Alternatively antibodies directed against a hormone or functional modifications of such antibodies may be used. Exemplary agents are described in detail herein. Those agents of use in the invention will preferably exhibit one or more of the following characteristics: 1) the ability to prevent, reduce or inhibit cell proliferation; 2) the ability to prevent, reduce or inhibit cell survival; 3) the ability to prevent, reduce or inhibit cell motility; 3) the ability to prevent, reduce or inhibit expression or activity of a hormone; 4) the ability to prevent, decrease, reduce or control metastasis of tumours. Preferably, suitable agents will exhibit two or more of these characteristics.

As shown herein, GH is encoded as a cellular factor that is expressed in certain cancer cells, and also by at least one subset of normal adult cells. Therefore, GH and related hormones, particularly hGH and variants thereof, hPRL, and hPL can be considered tumor-associated antigens. Several approaches can be used to target such hormones based on differences in expression and access in normal and cancer cells (reviewed, generally, in Paul, Fundamental Immunology, 1999, Lippincott-Raven Publishers, Philadelphia, Pa., Chapter 37). Cancer cells are likely to express these hormones at much higher levels and such differences in expression levels between normal and cancer cells can be exploited therapeutically (see, e.g., Brown J P, et al., Quantitative analysis of melanoma-associated antigen p97 in normal and neoplastic tissues. Proc Natl Acad Sci USA 1981; 78:539-543). Targeting may also be attained because of better access of hormone-specific effector cells to cancer cells than to normal cells. For example, an antigen expressed by cancer cells may be more available for binding due to incomplete glycosylation (e.g., as in the case of epithelial mucins). For cancer cells, increased expression of MHC molecules may make tumours a direct target for T cells (see, e.g., Uyttenhove C, et al. The expression of mouse gene PIA in testis does not prevent safe induction of cytolytic T cells against a P1A-encoded tumor antigen. Int J Cancer 1997; 70:349-356).

Multiple immunotherapeutic strategies involving innate or acquired immunity can be used to control cancer associated with GH and/or a related hormone, particularly hGH or variants thereof, hPRL, or hPL, including (a) local application of a live bacterial vaccine, e.g., BCG; (b) use of cytokines; (c) active immunization against one or more hormones; (d) passive therapy with antibodies to one or more hormones; and (e) adoptive transfer of effector cells (e.g., T cells). Active immunization against at least one hormone can be used to induce immune responses or passive immunization with a murine monoclonal antibody directed against GH and/or a related hormone, particularly hGH and/or variants thereof, hPRL, or hPL. For general guidance, see, e.g., Riethmüller G, et al., Monoclonal antibody therapy for resected Dukes' C colorectal cancer: seven-year outcome of a multicenter randomized trial. J Clin Oncol 1998; 16:1788-1794; Riethmüller G, et al., Randomized trial of monoclonal antibody for adjuvant therapy of resected Dukes' C colorectal carcinoma. German Cancer Aid 17-1A Study Group. Lancet 1994; 343:1177-1183; Herlyn D M, et al., Inhibition of growth of colorectal carcinoma in nude mice by monoclonal antibody. Cancer Res 1980; 40:717-721.

For immunization, pure antigens are often ineffective in inducing an acquired (i.e., antigen specific) immune response unless certain adjuvants are used to stimulate innate immunity. Therefore, numerous approaches are designed to stimulate innate immunity at the site of vaccinations by the use of chemical and/or bacterial agents. Synthetic peptides used in vaccines can be designed for particular MHC haplotypes (see, e.g., Toes R E et al. Peptide vaccination can lead to enhanced tumor growth through specific T-cell tolerance induction. Proc Natl Acad Sci USA 1996; 93:7855-7860). Antigenic peptides can be loaded onto heat-shock protein (or as recombinant virus-like particles) to increase the efficacy of immunization. Generally, effective induction of an immune response requires antigen presentation in an environment that provides appropriate help or secondary signals.

Several experimental designs use dendritic cells pulsed with virus-specific or tumor-associated peptides to induce tumor-reactive T cells and rejection of transplanted tumor cells. Dendritic cells can be loaded with synthetic antigenic peptides or recombinant proteins. Dendritic cells can also be loaded with one or more of: native peptides stripped from tumor cell surfaces; tumor-derived, peptide-loaded heat-shock proteins; tumor-derived mRNA; or fused tumor cells (for review, see Shurin M R. Dendritic cells presenting tumor antigen. Cancer Immunol Immunother 1996; 43:158-164). One advantage of these strategies is that powerful immunity can be induced to (unique) individually distinct tumor antigens, as well as tumor-associated antigens.

As further approaches, passive antibody therapy or adoptive transfer of tumor-specific T cells can be used. For example, passive immunization with an antibody for GH and/or a related hormone, particularly hGH and/or variants thereof, hPRL, or hPL can protect against challenge with tumor cells and can be therapeutic when given soon after challenge with the cancer cells (e.g., see Riethmüller G, et al. Monoclonal antibody therapy for resected Dukes' C colorectal cancer: seven-year outcome of a multicenter randomized trial. J Clin Oncol 1998; 16:1788-1794; Riethmüller G, et al. Randomized trial of monoclonal antibody for adjuvant therapy of resected Dukes' C colorectal carcinoma. German Cancer Aid 17-1A Study Group. Lancet 1994; 343:1177-1183; Herlyn D M, et al. Inhibition of growth of colorectal carcinoma in nude mice by monoclonal antibody. Cancer Res 1980; 40:717-721).

As alternate approaches, anti-idiotypic antibody treatment can be used to induce cancer cells to go into a long-lasting dormant state (see, e.g., Miller R A, Maloney D G, Warnke R, Levy R. Treatment of B-cell lymphoma with monoclonal anti-idiotype antibody. N Engl J Med 1982; 306:517-522). In addition, antibodies to tumour cells can be used as carriers for cytokines or cytotoxic agents, such as radiochemicals or natural toxins (see, e.g., Ghetie V, Vitetta E. Immunotoxins in the therapy of cancer: from bench to clinic. Pharmacol Ther 1994; 63:209-234; Reisfeld R A, Gillies S D. Recombinant antibody fusion proteins for cancer immunotherapy. Curr Top Microbiol Immunol 1996; 213:27-53). The recombinant antibody-cytokine or antibody-toxin fusion proteins may be used to concentrate these agents in the stroma surrounding the tumor cells. As alternatives, bispecific monoclonal antibodies can be engineered to bind effector cells as well as tumor antigens on the cancer cells. Monoclonal antibodies can also be humanized to reduce the stimulation of neutralizing anti-murine antibodies by patients. As still further approaches, adoptive transfer of T cells can be used with longer established tumor loads. T cells that have been isolated from patients can be expanded in vitro with IL-2 and then infused into patients who receive IL-2 as well (see, e.g., Smith C A, et al. Adoptive immunotherapy for Epstein-Barr virus-related lymphoma. Leuk Lymphoma 1996; 23:213-220).

The efficacy of an agent in inhibiting GH and/or a related hormone, particularly hGH and/or variants thereof, hPRL, or hPL, may be determined having regard to the description of the invention herein and known methodology. For example, efficacy of agents may be determined by observing their ability to prevent, reduce, or inhibit expression of a hormone, or one or more of the functional effects of the hormone. By way of particular example, the affect of the agent on one or more of cellular invasion, cellular migration, the level of gene transcription and hormone-responsive genes may be studied. Such studies may be conducted in vitro or in vivo.

The assays described herein after under the heading “Examples” may be used to determine the suitability of an agent in accordance with the invention. Specifically, RT-PCR and Northern blot analysis can be used to detect expression of GH and/or a related hormone, particularly hGH and/or variants thereof, hPRL, or hPL, at the mRNA level, and Western blotting and direct or indirect immunostaining can be used to detect the expression at the protein level. To detect hormone activity, cell-based assays for cell proliferation, cell survival, or cell motility can be used.

In respect of the effects of hormone inhibition on metastasis, an in vivo assay may be used, as described, for example, in Fidler, I. J. (1973) Nat. New Biol. 242, 148-149; and Price J. E. The biology of cancer metastasis. Prog. Clin. Biol. Res., 354A: 237-255,1990, or Kerbel R. S. What is the optimal rodent model for anti-tumor drug testing? Cancer Metastasis Rev., 17: 301-304,1998; Killion J. J., Radinsky R., Fidler I. J. Orthotopic models are necessary to predict therapy of transplantable tumours in mice. Cancer Metastasis Rev., 17: 279-284, 1998; and Price J. E. Analyzing the metastatic phenotype. J. Cell. Biochem., 56: 16-22, 1994.

### Administration Routes/Regimes

The inventors contemplate administration of any of the inhibitory agents or compositions of the invention by any means capable of delivering the desired activity (e.g., inhibition of GH and/or a related hormone, particularly hGH and/or variants thereof, hPRL, or hPL) to a target site within the body of a subject. Such agents and compositions can thereby be used to inhibit cell proliferation, cell survival, and/or cell motility at the target site. A target site may be any site within the body which may have or be susceptible to a disorder, and may include one or more cells, tissues or a specific tumor. For example, administration may include parenteral administration routes, systemic administration routes, oral and topical administration. Administration may be by way of injection, subcutaneous, intraorbital, ophthalmic, intraspinal, intracisternal, topical, infusion (using, e.g., slow release devices or minipumps such as osmotic pumps or skin patches), implant, aerosol, inhalation, scarification, intraperitoneal, intracapsular, intramuscular, intratumoral, intranasal, oral, buccal, transdermal, pulmonary, rectal or vaginal delivery. As will be appreciated, the administration route chosen may be dependent on the position of the target site within the body of a subject, as well as the nature of the agent or composition being used.

In a specific embodiment, in the case of polynucleotides, they may be administered for example by infection using defective or attenuated retroviral or other viral vectors (see e.g., U.S. Pat. No. 4,980,286), or by direct injection of naked DNA, or by use of microparticle bombardment (e.g., a gene gun; Biolistic, DuPont); by coating with lipids or cell-surface receptors or transfecting agents; encapsulation in liposomes, microparticles, or microcapsules; by linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors, and the like. In addition, nucleic acid-ligand complexes can be formed in which the ligand comprises a fusogenic viral peptide to disrupt endosomes, allowing the nucleic acid molecules to avoid lysosomal degradation.

In yet another embodiment, the polynucleotides can be targeted in vivo for cell specific uptake and expression, by targeting a specific receptor, as described for example in WO 92/06180 dated Apr. 16, 1992 (Wu et al.); WO 92/22635 dated Dec. 23, 1992 (Wilson et al.); WO 92/20316 dated Nov. 26, 1992 (Findeis et al.); WO 93/14188 dated Jul. 22, 1993 (Clarke et al.); and, WO 93/20221 dated Oct. 14, 1993 (Young). Alternatively, the polynucleotides can be introduced intracellularly and incorporated within host cell DNA for expression, by homologous recombination Koller and Smithies, 1989, Proc. Natl. Acad. Sci. USA 86:8932-8935; Zijlstra et al., 1989, Nature 342:435-438. Cells into which polynucleotides can be introduced for purposes of the present invention encompass any desired, available cell type. The appropriate cell type will depend on the nature of the disorder to be treated. However, by way of example, the polynucleotide can be introduced to a cancer cell.

As will be appreciated, the dose of an agent or composition administered, the period of administration, and the general administration regime may differ between subjects depending on such variables as the nature of the condition to be treated, severity of symptoms of a subject, the size of any tumour to be treated, the target site to be treated, the mode of administration chosen, and the age, sex and/or general health of a subject. Persons of average skill in the art to which the invention relates will readily appreciate or be able to determine appropriate administration regimes having regard to such factors. It should be appreciated that administration may include a single daily dose or administration of a number of discrete divided doses as may be appropriate. The inventors also contemplate the administration regimes which combine different modes or routes of administration. For example, intratumoural injection and systemic administration could be combined.

It should be appreciated that a method of the invention may comprise further steps such as the administration of additional agents or compositions which may be beneficial to a subject having regard to the condition to be treated. For example, other agents of use in treating proliferative disorders (such as the anti-neoplastic agents mentioned above) could be administered. It should be appreciated that such additional agents and compositions may be administered concurrently with the agents and compositions of the invention, or in a sequential manner. For example, the additional agents or compositions could be administered before or after administration of the agents or compositions of the invention. It should be appreciated in relation to sequential delivery of agents or compositions, that sequential administration of one agent or composition after the other need not occur immediately, although this may be preferable. There may be a time delay between delivery of the agents or compositions. The period of the delay will depend on factors such as the condition to be treated and the nature of the compositions or agents to be delivered. However, by way of example, the inventors contemplate periods of between hours to several days or months.

### Diagnostic Methods and Compositions

In one embodiment, the invention relates to use of one or more reagents of the invention in a method of diagnosing a disorder associated with cell proliferation, cell survival, or cell motility. Preferably, the method is for the diagnosis of a disorder characterised by aberrant cell proliferation, cell survival, or cell motility in subject. This aberrant cell proliferation, cell survival, or cell motility may occur in one or more cell type within a subject and can include metastatic disorders. Specific disorders include, for example, cancer (breast cancer, lung cancer, colon cancer, prostate cancer, or endometrial cancer, for example) and endometriosis. Examples of disorders include cancers such as adenocarcinoma, leukemia, lymphoma, melanoma, myeloma, sarcoma, teratocarcinoma, and cancers of the adrenal gland, bladder, bone, brain, breast, cervix, gall bladder, ganglia, gastrointestinal tract, heart, kidney, liver, lung, bone marrow, muscle, ovary, pancreas, parathyroid, penis, prostate, salivary glands, skin, spleen, testis, thymus, thyroid, and uterus. In various embodiments, the disorder is an epithelial tumor of the breast, lung, prostate, colon, pancreas, endometrium, stomach, or ovary, or a squamous cell carcinoma, or a melanoma, or a renal cancer or tumour. Specific breast, colon, and endometrial cancers are described in detail herein.

In accordance with the invention, antibodies which specifically bind GH and/or a related hormone, particularly hGH and/or variants thereof, hPRL, or hPL, may be used for the diagnosis of conditions or disorders characterized by expression of one or more of the hormones, or in assays to monitor patients being treated with hormone inhibitors. The antibodies useful for diagnostic purposes may be prepared in the same manner as those described above for therapeutics. Diagnostic assays for one or more hormones include methods which utilize the antibody and a label to detect GH and/or a related hormone, particularly hGH and/or variants thereof, hPRL, or hPL, in human body fluids or extracts of cells or tissues. The antibodies may be used with or without modification, and may be labeled by joining them, either covalently or non-covalently, with a reporter molecule. A wide variety of reporter molecules which are known in the art may be used, several of which are described above.

A variety of protocols, including ELISA, MA, and FACS, are known in the art and provide a basis for diagnosing altered or abnormal levels of expression of GH and/or a related hormone, particularly hGH or variants thereof, hPRL, or hPL. Normal or standard values for hormone expression are established by combining body fluids or cell extracts taken from normal mammalian subjects, preferably human, with antibody to one or more hormones under conditions suitable for complex formation. The amount of standard complex formation may be quantified by various methods, but preferably by photometric means. Quantities of hormone expressed in subject, control, and disease, samples from biopsied tissues are compared with the standard values. Deviation between standard and subject values establishes the parameters for diagnosing disorders.

In another embodiment of the invention, the polynucleotides encoding a hormone may be used for diagnostic purposes. The polynucleotides which may be used include oligonucleotide sequences, complementary RNA and DNA molecules, and PNAs. The polynucleotides may be used to detect and quantitate gene expression in biopsied tissues in which expression of one or more hormones may be correlated with disorders. The diagnostic assay may be used to distinguish between absence, presence, and excess expression of at least one hormone, and to monitor regulation of hormone levels during therapeutic intervention.

In one aspect, hybridization with PCR probes which are capable of detecting polynucleotide sequences, including genomic sequences, encoding GH and/or a related hormone, particularly hGH or variants thereof, hPRL, or hPL, may be used to identify nucleic acid sequences which encode the hormone. The specificity of the probe, whether it is made from a highly specific region, e.g., 10 unique nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding a hormone, alleles, or related sequences.

Probes may also be used for the detection of related sequences, and should preferably contain at least 50% of the nucleotides from any of the endogenous sequences. The hybridization probes of the subject invention may be DNA or RNA and derived from the nucleotide sequence of SEQ ID NO: 27-96, or fragments encompassing a nucleic acid sequence of SEQ ID NO: 27-96, or from genomic sequences including promoter, enhancer elements, and introns of the naturally occurring hormone.

Means for producing specific hybridization probes for DNAs encoding GH and/or a related hormone, particularly hGH and/or variants thereof, hPRL, or hPL, include the cloning of nucleic acid sequences encoding the hormone or modified sequences into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as ³²P or ³⁵S, or enzymatic labels, such as alkaline phosphatase coupled to the probe via avidin/biotin coupling systems, and the like.

Polynucleotide sequences encoding a hormone may be used for the diagnosis of disorders which are associated with either increased or decreased expression of the hormone. The polynucleotide sequences encoding the hormone may be used in Southern or northern analysis; dot blot or other membrane-based technologies; in PCR technologies; or in dipstick, pin, ELISA assays; or microarrays utilizing fluids or tissues from patient biopsies to detect altered hormone expression. Such qualitative or quantitative methods are well known in the art.

In a particular aspect, the nucleotide sequences encoding GH or a related hormone, particularly hGH or variants thereof, hPRL, or hPL may be useful in assays that detect activation or induction of various cancers, particularly those mentioned above. The nucleotide sequences encoding a hormone may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the nucleotide sequences have hybridized with nucleotide sequences in the sample, and the presence of altered levels of nucleotide sequences encoding the hormone in the sample indicates the presence of the associated disorder. Such assays may also be used to evaluate the efficacy of a particular therapeutic treatment regimen in animal studies, in clinical trials, or in monitoring the treatment of an individual patient.

In order to provide a basis for the diagnosis of a disorder associated with expression of GH and/or a related hormone, particularly hGH or variants thereof, hPRL, or hPL, a normal or standard profile for expression is established. This may be accomplished by combining body fluids or cell extracts taken from normal subjects, either animal or human, with a sequence, or a fragment thereof, which encodes GH, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for a disorder. Deviation between standard and subject values is used to establish the presence of the disorder.

Once the disorder is diagnosed and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months. With respect to cancer, the presence of a relatively high amount of transcript in biopsied tissue from an individual may indicate a predisposition for the development of the disorder, or may provide a means for detecting the disorder prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding GH may involve the use of PCR. Such oligomers may be chemically synthesized, generated enzymatically, or produced in vitro. Oligomers will preferably consist of two nucleotide sequences, one with sense orientation (5′.fwdarw.3′) and another with antisense (3′.fwdarw.5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of GH and/or a related hormone, particularly hGH and/or variants thereof, hPRL, or hPL, include radiolabeling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 229-236). The speed of quantitation of multiple samples may be accelerated by running the assay in an ELISA format where the oligomer of interest is presented in various dilutions and a spectrophotometric or colorimetric response gives rapid quantitation.

In further embodiments, oligonucleotides or longer fragments derived from any of the polynucleotide sequences described herein may be used as targets in a microarray. The microarray can be used to monitor the expression level of large numbers of genes simultaneously (to produce a transcript image), and to identify genetic variants, mutations and polymorphisms. This information may be used to determine gene function, to understand the genetic basis of the disorder, to diagnose the disorder, and to develop and monitor the activities of therapeutic agents. In one embodiment, the microarray is prepared and used according to the methods known in the art such as those described in PCT application WO 95/11995 (Chee et al.), Lockhart, D. J. et al. (1996; Nat. Biotech. 14: 1675-1680) and Schena, M. et al. (1996; Proc. Natl. Acad. Sci. 93: 10614-10619).

The microarray is preferably composed of a large number of unique, single stranded nucleic acid sequences, usually either synthetic antisense oligonucleotides or fragments of cDNAs, fixed to a solid support. The oligonucleotides are preferably about 6 to 60 nucleotides in length, more preferably about 15 to 30 nucleotides in length, and most preferably about 20 to 25 nucleotides in length. For a certain type of microarray, it may be preferable to use oligonucleotides which are 7 to 10 nucleotides in length. The microarray may contain oligonucleotides which cover the known 5′ or 3′ sequence, or may contain sequential oligonucleotides which cover the full length sequence; or unique oligonucleotides selected from particular areas along the length of the sequence.

Polynucleotides used in the microarray may be oligonucleotides that are specific to a gene or genes of interest in which at least a fragment of the sequence is known or that are specific to one or more unidentified cDNAs which are common to a particular cell or tissue type or to a normal, developmental, or disease state. In certain situations, it may be appropriate to use pairs of oligonucleotides on a microarray. The pairs will be identical, except for one nucleotide preferably located in the centre of the sequence. The second oligonucleotide in the pair (mismatched by one) serves as a control. The number of oligonucleotide pairs may range from 1 to 1,000,000.

In order to produce oligonucleotides to a known sequence for a microarray, the gene of interest is examined using a computer algorithm which starts at the 5′ or more preferably at the 3′ end of the nucleotide sequence. The algorithm identifies oligomers of defined length that are unique to the gene, have a GC content within a range suitable for hybridization, and lack predicted secondary structure that may interfere with hybridization. In one aspect, the oligomers are synthesized at designated areas on a substrate using a light-directed chemical process. The substrate may be paper, nylon or any other type of membrane, filter, chip, glass slide, or any other suitable solid support.

In one aspect, the oligonucleotides may be synthesized on the surface of the substrate by using a chemical coupling procedure and an ink jet application apparatus, such as that described in PCT application WO 95/251116 (Baldeschweiler et al.). In another aspect, a gridded array analogous to a dot or slot blot (HYBRIDOT apparatus, Life Technologies) may be used to arrange and link cDNA fragments or oligonucleotides to the surface of a substrate using a vacuum system, thermal, UV, mechanical or chemical bonding procedures. In yet another aspect, an array may be produced by hand or by using available devices, materials, and machines (including multichannel pipettors or robotic instruments; Brinkmann, Westbury, N.Y.) and may include about 8, 24, 96, 384, 1536 or 6144 oligonucleotides, or any other multiple from 2 to 1,000,000, which lends itself to the efficient use of commercially available instrumentation.

In order to conduct sample analysis using the microarrays, polynucleotides are extracted from a biological sample. The biological samples may be obtained from any bodily fluid (e.g., blood, urine, saliva, phlegm, gastric juices, etc.), cultured cells, biopsies, or other tissue preparations. To produce probes, the polynucleotides extracted from the sample are used to produce nucleic acid sequences which are complementary to the oligonucleotides on the microarray. If the microarray consists of cDNAs, antisense RNAs are appropriate probes. Therefore, in one aspect, mRNA is used to produce cDNA which, in turn and in the presence of fluorescent nucleotides, is used to produce fragment or oligonucleotide antisense RNA probes. These fluorescently labeled probes are incubated with the microarray so that the probe sequences hybridize to the cDNA oligonucleotides of the microarray. In another aspect, nucleic acid sequences used as probes can include polynucleotides, fragments, and complementary or antisense sequences produced using restriction enzymes, PCR technologies, and oligolabeling kits (Amersham Pharmacia Biotech), which are well known in the area of hybridization technology.

Incubation conditions are adjusted so that hybridization occurs with precise complementary matches or with various degrees of less complementarity. After removal of non-hybridized probes, a scanner is used to determine the levels and patterns of fluorescence. The scanned images are examined to determine degree of complementarity and the relative abundance of each oligonucleotide sequence on the microarray. A detection system may be used to measure the absence, presence, and amount of hybridization for all of the distinct sequences simultaneously. This data may be used for large scale correlation studies or functional analysis of the sequences, mutations, variants, or polymorphisms among samples (Heller, R. A. et al., (1997) Proc. Natl. Acad. Sci. 94:2150-55).

In another embodiment of the invention, the nucleic acid sequences which encode a hormone may be used to generate hybridization probes which are useful for mapping the naturally occurring genomic sequence. The sequences may be mapped to a particular chromosome, to a specific region of a chromosome, or to artificial chromosome constructions, such as human artificial chromosomes (HACs), yeast artificial chromosomes (YACs), bacterial artificial chromosomes (BACs), bacterial P1 constructions or single chromosome cDNA libraries (cf. Price, C. M. (1993) Blood Rev. 7:127-134; Trask, B. J. (1991) Trends Genet. 7:149-154).

Fluorescent in situ hybridization (FISH as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in various scientific journals or at the Online Mendelian Inheritance in Man (OMIM) site. Correlation between the location of the gene encoding a hormone on a physical chromosomal map and a specific disorder, or predisposition to a specific disorder, may help delimit the region of DNA associated with that disorder. The nucleotide sequences of the subject invention may be used to detect differences in gene sequences between normal, carrier, and affected individuals.

In situ hybridization of chromosomal preparations and physical mapping techniques, linkage analysis using established chromosomal markers, may be used to extend genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disorder has been crudely localized by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequence of the subject invention may also be used to detect differences in the chromosomal location due to translocation, inversion, etc., among normal, carrier, and affected individuals.

In another embodiment of the invention, GH or a related hormone, particularly hGH or variants thereof, hPRL, or hPL, or its functional or immunogenic fragments or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between the hormone and the agent being tested, may be measured. In a further embodiment, one may use competitive drug screening assays in which neutralizing antibodies specifically compete with a test compound for binding to the hormone. In this same manner, an antibody of the invention can be used to detect the presence of any amino acid sequence which shares one or more antigen binding sites with the antibody.

Another technique for drug screening which may be used provides for high throughput screening of compounds having suitable binding affinity to the protein of interest as described in WO 84/03564. In this method, as applied to one or more hormones, large numbers of different small test compounds are synthesized on a solid substrate, such as plastic pins or some other surface. The test compounds are reacted with the hormone, or fragments thereof, and washed. Bound GH is then detected by methods well known in the art. Purified hormone can also be coated directly onto plates for use in the aforementioned drug screening techniques. Alternatively, non-neutralizing antibodies can be used to capture the peptide and immobilize it on a solid support.

In additional embodiments, the nucleotide sequences which encode GH or a related hormone, particularly hGH or variants thereof, hPRL, or hPL, may be used in any molecular biology techniques that have yet to be developed, provided the new techniques rely on properties of nucleotide sequences that are currently known, including, but not limited to, such properties as the triplet genetic code and specific base pair interactions.

### Kits for Treatment or Diagnosis

The agents and compositions of the invention may be used in kits suitable for controlling or inhibiting GH and/or a related hormone, particularly hGH and/or variants thereof, hPRL, or hPL, for the treatment of a disorder as defined herein. In other aspects, the agents and compositions may be used in diagnostic kits. Kits can comprise at least one agent of the invention in a suitable container. The agents may be formulated suitable for direct administration to a subject for example, as agents or pharmaceutical compositions. Alternatively, the kit may comprise one or more agents in one container and pharmaceutical diluents, carriers and/or excipients in another; the contents of each container being mixed together prior to administration. The kit may also comprise additional agents and compositions in further separate containers as may be necessary for a particular application. Any container suitable for storing and/or administering an agent or composition may be used in a kit of the invention. Suitable containers will be appreciated by persons skilled in the art. By way of example, such containers include vials and syringes. The containers may be suitably sterilised and hermetically sealed. Further, kits of the invention can also comprise instructions for the use and administration of the components of the kit. The invention is further elucidated with reference to the examples below.

### EXAMPLES

The examples described herein are for purposes of illustrating embodiments of the invention. Other embodiments, methods, and types of analyses are within the scope of persons of ordinary skill in the molecular diagnostic arts and need not be described in detail herein. Other embodiments within the scope of the art are considered to be part of this invention.

### Example 1

### Materials and Methods

### Cell Lines and Cell Transfection

The human endometrial carcinoma cell lines, RL95-2 and AN3, were obtained from the American Type Culture Collection (ATCC). RL95-2 cell were grown in Dulbecco's modified Eagle's medium and Ham's F12 medium (1:1) with 10 mM HEPES and 2.0 g/L sodium bicarbonate supplemented with 10% fetal bovine serum, 100 IU/ml penicillin, 100 μg/ml streptomycin, 2 mM L-glutamine and 0.005 mg/ml insulin. AN3 cells were grown in minimum essential medium (Eagle) with 2 mM L-glutamine and Earle's BSS adjusted to contain 1.5 g/L sodium bicarbonate, supplemented with 10% fetal bovine serum, 100 IU/ml penicillin, 100 μg/ml streptomycin, 0.1 mM non-essential amino acids and 1.0 mM sodium pyruvate. Growth of both cell lines was maintained in at 37° C. in a humidified 5% CO₂ atmosphere.

The plasmid pcDNA3-hGH was constructed by cloning a BamHI fragment derived from the vector pMT-hGH (Palmiter R D, Norstedt G, Gelinas R E, Hammer R E, Brinster R L (1983). Metallothionein-human GH fusion genes stimulate growth of mice. Science 222: 809-14), containing the entire hGH gene, into pcDNA3. For generation of stable cell lines, RL95-2 and AN3 cells were stably transfected with plasmids pcDNA3 or pcDNA3-hGH using FuGENE® 6 (Roche Applied Science, USA). Stable transfectants were selected by incubation in 800 μg/ml G418 for 14 days as described previously (Moller C, Hansson A, Enberg B, Lobie P E, Norstedt G (1992). Growth hormone (GH) induction of tyrosine phosphorylation and activation of mitogen-activated protein kinases in cells transfected with rat GH receptor cDNA. J Biol Chem 267: 23403-8). RL95-2 and AN3 cells stably transfected with pMT-hGH were designated RL95-2-hGH and AN3-hGH, respectively, whereas cells stably transfected with pcDNA3 were designated RL95-2-VECTOR and AN3-VECTOR.

siRNA constructs sihGH5 and sihGH6 were generated by cloning. The sihGH5 construct comprised the following target sequence: AAGTATTCATTCCTGCAGAAC (SEQ ID NO: 97), directed towards human growth hormone 1 and human growth hormone 2. The sihGH6 construct comprised the following target sequence: AACAGCCTGGTGTACGGCGCC (SEQ ID NO: 98) directed towards human growth hormone 1. For generation of stable cell lines, RL95-2 cells were stably transfected with plasmids sihGH5, sihGH6 of sivector using FuGENE® 6 (Roche Applied Science, USA). Stable transfectants were selected by incubation in 800 μg/ml G418 for 14 days as described previously. RL95-2 cells stably transfected with sihGH5 were designated RL95-2-sihGH5, cells stably transfected with sihGH6 were designated RL95-2-hGH6, and cells stably transfected with vector only were designated RL95-2-sivector.

### Total Cell Number Assay

Cell lines were seeded in triplicate in six-well plates at a density of 5×10⁴ cellsper well in either medium containing 10% FBS or in serum-deficit media (0.2% FBS). Growth of cells was maintained at 37° C. in 5% CO₂. On the days indicated, cells were trypsinized with 0.5% trypsin and the cell number was determined using a hematocytometer (Zhu Z, Mukhina S, Zhu T, Mertani H C, Lee K O, Lobie P E (2005). p44/42 MAP kinase-dependent regulation of catalase by autocrine human growth hormone protects human mammary carcinoma cells from oxidative stress-induced apoptosis. Oncogene 24: 3774-85). Experiments were performed in triplicate and each assay was performed at least twice.

### 5′-Bromo-2′-Deoxyuridine Incorporation Assay

Mitogenesis was directly assayed by measuring incorporation of 5′-bromo-2′-deoxyuridine (BrdU) during DNA synthesis (Sawa T, Sasaoka T, Hirai H, Ishihara H, Ishiki M, Wada T et al (1999). Intracellular signalling pathways of okadaic acid leading to mitogenesis in Rat1 fibroblast overexpressing insulin receptors: okadaic acid regulates Shc phosphorylation by mechanisms independent of insulin. Cell Signal 11: 797-803). For incorporation of 5′-bromo-2′-deoxyuridine (BrdU), stable cells were washed twice with PBS, trypsinised and seeded to into six well plates in either medium containing 10% FBS or in serum-deficit media (0.2% FBS). Both RL95-2 and AN3 cells were pulse-labeled with 20 μm BrdU for 45 min, washed twice with PBS, and fixed in cold 4% paraformaldehyde for 30 min. BrdU detection was performed using the BrdU staining kit from (VECTASTAIN Elite ABC Kit, Invitro Tech, NZ) according to the manufacturer's instructions. A total population of over >5000 cells was analysed in several arbitrarily chosen microscopic fields to determine the BrdU labeling index (percentage of cells synthesizing DNA). Experiments were performed in triplicate and each assay was performed at least twice.

### Measurement of Apoptosis

Apoptotic cell death was measured by the terminal deoxynucleotidyl transferase biotin-dUTP nick end labeling (TUNEL) assay. Sub-confluent stable cells seeded in six well plates were incubated in serum-free medium. After a culture period of 48 hr in the serum-free medium, cells were trypsinized with 0.5% trypsin and washed twice with 1×PBS. TUNEL detection using a (APO-BrdU™ TUNEL Assay, Invitrogen, NZ) kit was performed as per manufacturer's instructions. The population of TUNEL positive cells was determined using a hemocytometer and was expressed as a percentage of the total number of cells. Experiments were performed in triplicate and each assay was performed at least twice. Caspase 3/7 activity to indicate onset of apoptosis was measured using a kit from Promega according to the manufacturer's instructions.

### Cell Behaviour and Morphogenesis Assays in Matrigel

Tissue culture dishes were coated with Matrigel (BD Bioscience, Franklin Lakes, N.J.) at 37° C. for 30 minutes before addition of stable cell lines. The behaviour of cell lines was assessed and digitally recorded at 24 hour intervals using an inverted light microscope (Olympus 1×2-ILL100, Japan).

### Anchorage-Independent Growth Assays

Anchorage-independent growth assays, including suspension culture, were performed as previously described (Zhang X, Zhu T, Chen Y, Mertani H C, Lee K O, Lobie P E (2003). Human growth hormone-regulated HOXA1 is a human mammary epithelial oncogene. J Biol Chem 278: 7580-90). Stable cells (5×10⁵) were seeded into 75 cm² flasks in monolayers in wild type medium. After 72 h, cells were trypsinized with 0.5% trypsin, and the cell number was determined using a hematocytometer. For suspension culture, cells (5×10³) were grown in Poly-HEMA (Sigma) coated six well plates. On the days indicated cells were harvested and counted using a hematocytometer. For soft agar colony formation, stable cells were cultured in six-well plates first covered with an agar layer (serum-free medium with 0.5% agar). The middle layer contained 5×10³ cells in media with 10% FBS and 0.35% agar. Serum containing medium was added as the top layer to prevent drying of the agarose gels. The plates were incubated for 18 days, after which the cultures were inspected and photographed. For foci formation, cells (5×10³) were seeded in to six well plates in monolayers in media containing 10% FBS. Cells were incubated for 20 days after which the cultures were inspected and photographed.

### In Vitro Cell Migration and Invasion Assays

Assays were performed in BD BioCoat Matrigel invasion chambers according to the manufacturer's instructions, with uncoated porous filters (8 μm pore size) for estimation of cell migration and filters precoated with Matrigel to examine cell invasion. For invasion assays, the filters were coated with 500 μl growth factor-reduced Matrigel (1:5, Matrigel and serum-free DMEM-F12 media). Prior to experimentation, cells were serum deprived for 24 hours. Cells (5×10⁴) in 50 μl of serum-free DMEM-F12 were placed on each filter, and 10% FBS containing DMEM-F12 conditioned medium was placed in the lower chamber as a chemo-attractant. After incubation for 48 h, the filters were fixed with 4% paraformaldehyde, and stained with crystal violet. Cells on the upper surface of the filters were removed with cotton swabs. Cells that had invaded to the lower surface of the filter were counted by microscopy selecting over eight random fields per filter (10× magnification). Values for cell migration or invasion were expressed as the average number of cells per microscopic field per one filter in triplicate. Experiments were repeated three times to authenticate the results. For the wound migration assay, confluent monolayers of stable transfected cell lines were scraped with pipette tips, washed with PBS, and incubated in culture medium supplemented with 10% FBS for 6 days.

### ELISA

Stable cell lines were grown to confluence in six-well plates. Cells were washed with phosphate-buffered saline (PBS), pH 7.4 and the medium was then changed to serum-free medium for 24 h. The amount of hGH produced and secreted over a 24-h period into 1 ml of serum-free medium was then estimated. An ELISA for the quantitation of hGH was performed using an hGH coated-well ELISA kit (Diagnostic Systems Laboratories Inc., USA) according to the manufacturer's instructions.

### Cell Viability

Cell viability was determined using the MTT assay as previously described (van de Loosdrecht A A, Beelen R H, Ossenkoppele G J, Broekhoven M G, Langenhuijsen M M (1994). A tetrazolium-based colorimetric MTT assay to quantitate human monocyte mediated cytotoxicity against leukemic cells from cell lines and patients with acute myeloid leukemia. Immunol Methods 174: 311-20). Briefly, cells (1×10⁴ cells per well) were seeded into 96-well microtiter plates in a final volume of 200 μl 10% FBS containing or serum-deficit media (0.2% FBS). Cells were incubated at 37° C., 5% CO₂ for the indicated time periods, following which 20 μl of MTT reagent (5 mg/ml in PBS) was added to each well and the plates were incubated for an additional 4 h at 37° C. 100 μl lysis reagent (10% SDS with 12N HCl) was added to each well and the absorbance at 595 nm read the following day. Each experiment was carried out in triplicate and repeated at least twice to confirm results.

hGH-antisera Treatments:

hGH-antisera were purchased from National Hormone & Peptide Program (California, USA). These hGH-antisera were collected form rabbit. Cells were treated with different amount of hGH-antisera. An equivalent concentration of normal rabbit serum (NRS) was added to the medium of control wells.

### Production of New Polyclonal Antibodies to Hgh

Immunization: Chickens were immunized with recombinant human growth hormone (−0.5 mg/chicken) with complete Freund's adjuvant on. Day 0 and boosted again on day 9, 29, and 39 (−0.17 mg/chicken). Eggs were collected pre- and post-immunizations.

IgY extraction from egg yolk: The eggs were collected and egg yolk separated and pooled. The purification was performed at 4° C. Egg yolk was diluted and centrifuged. IgY was precipitated from the clear supernatant. The precipitate was separated by centrifugation and dissolved in PBS. The pre- and post-immunization chicken IgY samples were filter sterilised to yield a final concentration of ˜5 mg/ml with purity equal or greater than 90% and stored at 4° C.

Preparation of affinity column: Affigel-15 (Bio-Rad 153-6052) bead is a succinimide linker bound matrix with a free carboxylic group ready to bind primary amines of amino acids present in the proteins. The beads were washed in cold water, resuspended in binding buffer (10 mM MOPS, pH 7.5). Human growth hormone (antigen) was dissolved in water and dialysed overnight against binding buffer. Antigen and Affigel-15 beads were mixed gently for 4 hr. After this, 1 M ethanolamine-HCl, pH 8.0 was added to facilitate the protein binding and agitated for 1 hr. Antigen bound beads were then transferred to an EconoColumn (Bio-Rad), and subjected to a series of washes. First, beads were washed using binding buffer, followed by PBS, then by glycine-HCl pH 2.4/150 mM NaCl, and then again by PBS.

Anti-hGH-IgY purification: IgY was applied to the prepared hGH affinity column, twice. The column was washed with PBS then anti-hGH-IgY was eluted using 100 mM glycine-HCl pH 2.4/150 mM NaCl. The eluted fractions were immediately neutralized with 1 M Tris-HCl, pH 8.0. The anti-hGH-IgY was then dialysed against PBS, pH 7.4 for 48 hr at 4° C. and concentrated to equal or more than 4 mg/ml.

### Real-Time PCR and Reverse Transcription-PCR

Extraction of total RNA and RT-PCR assay were done as previously described (Mertani H C, Zhu T, Goh E L, Lee K O, Morel G, Lobie P E (2001). Autocrine human growth hormone (hGH) regulation of human mammary carcinoma cell gene expression. Identification of CHOP as a mediator of hGH-stimulated human mammary carcinoma cell survival. J Biol Chem 276: 21464-75). Sequences of the oligonucleotide primer pairs used for RT-PCR were as follows: For hGH: 5′-CCG-ACA-CCC-TCC-AAC-AGG-GA-3′ (SEQ ID. NO: 61) and 5′-CCT-TGT-CCA-TGT-CCT-TCC-TG-3′ (SEQ. ID NO: 62); for hGHR: 5′-CTC-AAC-TGG-ACT-TTA-CTG-AAC-G-3′ (SEQ ID NO: 63) and 5′-AAT-CTT-TGG-AAC-TGG-AAC-TGG-G-3′ (SEQ. ID NO: 64); and for 13-Actin: 5′-ATG ATA TCG CCG CGC TCG-3′ (SEQ ID NO: 65) and 5′-CGC TCG GTGAGG ATC TTC A-3′ (SEQ ID NO: 66).

For real-time PCR, total RNA was converted to cDNA using SuperScript™ III First-Strand Synthesis SuperMix for qRT-PCR (Invitrogen, CA) as per manufacture's instructions. The ABI 7700® real-time PCR system (Applied Biosystems, USA) was used for analysis. Multiple gene markers distributed around the genome and three housekeeping genes were used for real-time PCR analysis using the SYBR® GreenER™ qPCR SuperMix for ABI PRISM® (Invitrogen, CA). The sequence information of all the primers was listed in Table 6 (below).

TABLE 6
Primer pairs for real-time PCR

| | | | |
|---|---|---|---|
| | | Amplicon | SEQ |
| Gene | Primer Sequence | size | ID NO: |

| | | | | |
|---|---|---|---|---|
| Beta-actin | Forward: | TTCCTGGGCATGGAGTC | 84 bp | 67 |
| | Reverse: | CAGGTCTTTGCGGATGTC | | 68 |
| HPRT | Forward: | TGACACTGGCAAAACAATGCA | 94 bp | 69 |
| | Reverse: | GGTCCTTTTCACCAGCAAGCT | | 70 |
| GAPDH | Forward: | TGCACCACCAACTGCTTAGC | 87 bp | 71 |
| | Reverse: | GGCATGGACTGTGGTCATGAG | | 72 |
| Fibronectin 1(FN1) | Forward: | CCCATCAGCAGGAACACCTT | 84 bp | 73 |
| | Reverse: | GGCTCACTGCAAAGACTTTGAA | | 74 |
| Alpha-catenin | Forward: | CCATGCAGGCAACATAAACTTC | 105 bp | 75 |
| | Reverse: | AGGGTTGTAACCTGTGTAACAAG | | 76 |
| Beta-catenin | Forward: | CCTATGCAGGGGTGGTCAAC | 95 bp | 77 |
| | Reverse: | CGACCTGGAAAACGCCATCA | | 78 |
| E-cadherin | Forward: | CCCACCACGTACAAGGGTC | 94 bp | 79 |
| | Reverse: | CTGGGGTATTGGGGGCATC | | 80 |
| GH 1 | Forward: | CAGGGAGGAAACACAACAGAAA | 155 bp | 81 |
| | Reverse: | TTAGGAGGTCATAGACGTTGCT | | 82 |
| GH receptor | Forward: | GCTAACTAGCAATGGTGGTACAG | 103 bp | 83 |
| | Reverse: | GACGTTCAGTAAAGTCCAGTTGA | | 84 |
| HOX-A1 Homeobox | Forward: | CGTGAGAAGGAGGGTCTCTTG | 147 bp | 85 |
| | Reverse: | GTGGGAGGTAGTCAGAGTGTC | | 86 |
| CCND1 (Cyclin D1) | Forward: | ACGAAGGTCTGCGCGTGTT | 323 bp | 87 |
| | Reverse: | CCGCTGGCCATGAACTACCT | | 88 |
| TP53 | Forward: | TGCAGCTGTGGGTTGATTCC | 396 bp | 89 |
| | Reverse: | AAACACGCACCTCAAAGCTGTTC | | 90 |
| BCL2 | Forward: | TCCGCATCAGGAAGGCTAGA | 113 bp | 91 |
| | Reverse: | AGGACCAGGCCTCCAAGCT | | 92 |
| BAD | Forward: | CCCAGAGTTTGAGCCGAGTG | 249 bp | 93 |
| | Reverse: | CCCATCCCTTCGTCGTCCT | | 94 |
| BAK1 | Forward: | GAACAGGAGGCTGAAGGGGT | 307 bp | 95 |
| | Reverse: | TCAGGCCATGCTGGTAGACG | | 96 |

For the reaction, 5 ng total cDNA from both test genes and endogenous control genes was added to a 20 μl mixture containing SYBR® GreenER™ qPCR SuperMix for ABI PRISM®, and 200 nM each ofprimers. Triplicate reactions were performed for each marker in a 384-well plate using a two-step amplification program of initial denaturation at 95° C. for 10 min, followed by 40 cycles of 95° C. for 20 s and 60° C. for 30 s. A melting curve analysis step was carried out at the end of the amplification, consisting of denaturation at 95° C. for 1 min and re-annealing at 55° C. for 1 min. Standard curves were generated from each experimental plate using serial 5-fold dilutions of untreated cDNA.

The Ct for each reaction was calculated using the ABI software. Amplification efficiencies were calculated according to the equation E=10^{(-1/slope)} (Heid C A, Stevens J, Livak K J, Williams P M (1996). Real time quantitative PCR. Genome Res 6: 986-94) and ranged from 90-104% for all gene markers; no unspecific amplification or primer dimmer was observed in any of the reactions as confirmed by the melt curve analysis. To compensate for potential differences in E between markers, the relative expressions was computed, based on the efficiency (E), normalized by a panel of housekeeping genes, β-actin, HPRT, and GAPDH and the Ct difference (Δ) of sample versus control (ΔCt_{target-control}). Relative expression=2^{-(Ct,Target-Ct,HKG)-(Ct, Control-Ct,HKG)}; Relative expression=2^{-ddCt}

### Statistics

All data are expressed as means±SD of triplicates. All experiments were repeated at least two times. Data were analysed using the two-tailed t-test or ANOVA.

### Example 2

### Results

### Forced Expression of hGH in Endometrial Cells Results in Increased Total Cell Number

Autocrine production of hGH has previously been demonstrated to affect cellular function and increase oncogenicity in a mammary carcinoma cell model (Kaulsay K K, Zhu T, Bennett W, Lee K O, Lobie P E (2001). The effects of autocrine human growth hormone (hGH) on human mammary carcinoma cell behaviour are mediated via the hGH receptor. Endocrinology 142: 767-77, Mukhina S, Mertani H C, Guo K, Lee K O, Gluckman P D, Lobie P E (2004). Phenotypic conversion of human mammary carcinoma cells by autocrine human growth hormone. Proc Natl Acad Sci USA 101: 15166-71, Zhu T, Starling-Emerald B, Zhang X, Lee K O, Gluckman P D, Mertani H C et al (2005). Oncogenic transformation of human mammary epithelial cells by autocrine human growth hormone. Cancer Res 65: 317-24). To determine whether autocrine hGH affected cellular function in an endometrial carcinoma model, RL95-2 cells were stably transfected with an expression plasmid encoding the hGH gene (RL95-2-hGH). As a control, a second cell line stably transfected with vector (RL95-2-vector) was established in parallel. Pooled stable transfectants were used in order to minimize any effect of potential clonal selection.

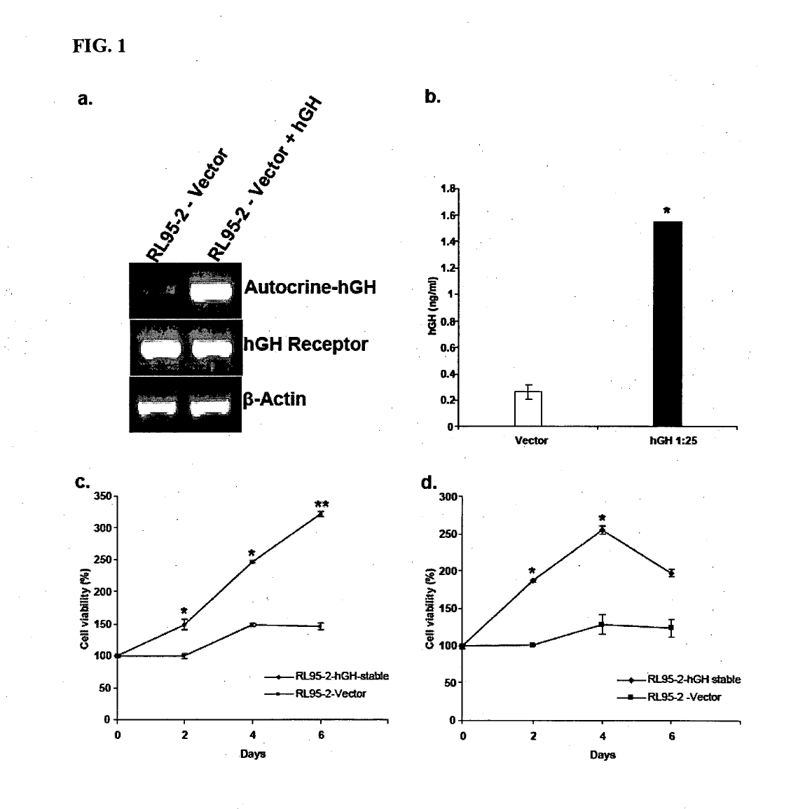

RL95-2-hGH stable cells synthesized hGH mRNA as demonstrated by RT-PCR (FIG. 1a) and secreted hGH protein into the extracellular medium as demonstrated by ELISA analysis (FIG. 1b). This analysis also indicated that RL95-2 cells endogenously express hGH transcripts and hGH protein at low levels (FIGS. 1a and b). Real-time PCR relative quantification analysis was also conducted to determine the levels of hGH and the hGH receptor in stably transfected cell lines. This analysis demonstrated a 3×10² fold increase of autocrine-hGH, and no change of hGH-receptor transcripts in the RL95-2-hGH cell line when compared with RL95-2-vector cells.

The continued expression of the hGH-receptor mRNA in stably transfected cell lines was also demonstrated by RT-PCR (FIG. 1a). No change in receptor expression was observed in hGH-expressing stable cells (RL95-2-hGH) when compared with the control cell line (RL95-2-vector). Neither the RL95-2-vector nor RL95-2-hGH cells showed detectable levels of IGF-1 mRNA by real-time PCR, indicating that the autocrine hGH-mediated responses reported below are not due to IGF-1 effects on cell function. However, real-time PCR demonstrated that IGF-2 mRNA was increased in RL95-2-hGH cells when compared with the RL95-2-vector cell line.

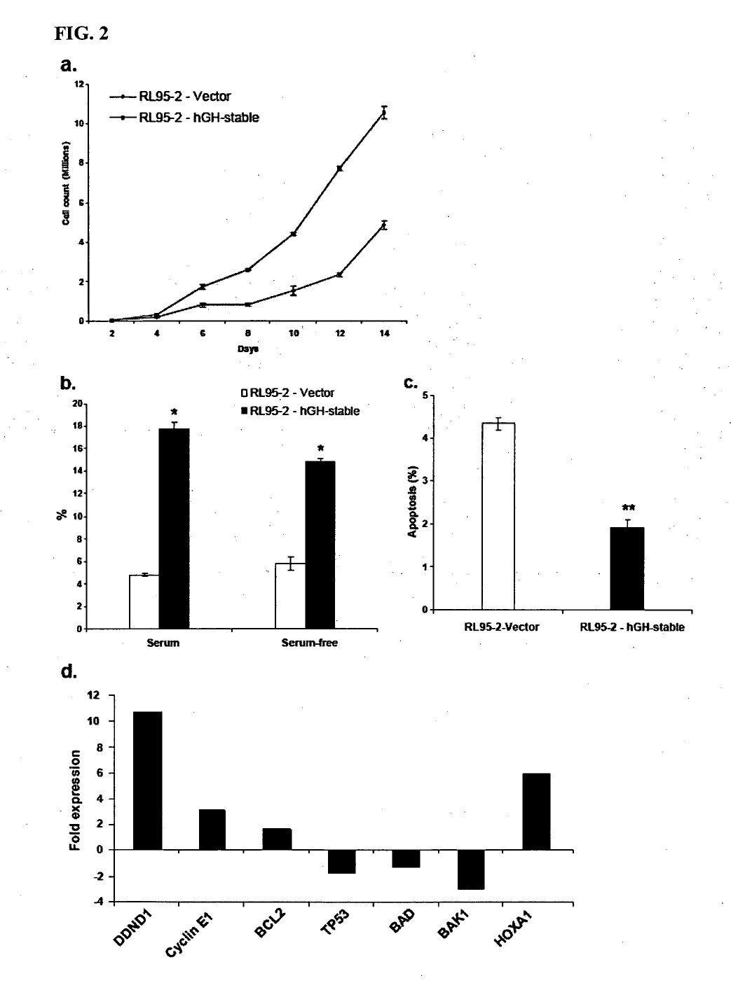

To determine the effect of autocrine of hGH on RL95-2 cellular function, the proliferation of the stably transfected RL95-2 cell lines in serum and serum-deficit medium was evaluated. Assessment of cell viability and growth using an MTT assay demonstrated that autocrine hGH expression in RL95-2-hGH cells resulted in increased viability when compared with RL95-2-vector in both serum containing and serum-deficient medium (FIGS. 1c and d). Total cell number assays conducted in 10% serum medium demonstrated that RL95-2-hGH cell number increased dramatically faster than RL95-2-vector cell number over a period of 6 days despite an identical original plating density (FIG. 2a). Increased cell growth may result from the net effect of increased proliferation and/or a decrease in apoptosis. Autocrine expression of hGH significantly increased cell cycle progression as determined by BrdU incorporation in RL95-2-hGH cells in serum and serum free conditions (FIG. 2b). In addition, autocrine hGH reduced apoptotic cell death consequent to serum deprivation (FIG. 2c) when compared to the control cell line, RL95-2-vector.

Transcriptional regulation of genes required for cell cycle progression and cell survival are directly associated with activation of signalling pathways involved in oncogenic transformation of cells (Evan G, Littlewood T (1998). A matter of life and cell death. Science 281: 1317-22, Nagasawa H, Noguchi Y, Mori T, Niki K, Namiki H (1985). Suppression of normal and preneoplastic mammary growth and uterine adenomyosis with reduced growth hormone level in SHN mice given monosodium glutamate neonatally. Eur J Cancer Clin Oncol 21: 1547-51). Autocrine hGH has previously been demonstrated to differentially regulate gene expression in MCF-7 cells (Xu X Q, Emerald B S, Goh E L, Kannan N, Miller L D, Gluckman P D et al (2005). Gene expression profiling to identify oncogenic determinants of autocrine human growth hormone in human mammary carcinoma. J Biol Chem 280: 23987-4003). Real-Time PCR analysis was utilized to determine the mRNA levels of several key genes involved in cell proliferation and apoptosis. Forced expression of hGH in RL95-2 cells up-regulated the mRNA levels of DDND1 (Cyclin D1) and Cyclin E1, required for cell cycle progression and Bcl-2, an anti-apoptotic gene required for cell survival. mRNA levels of genes encoding pro-apoptotic proteins p53 (TP53), BAD, and BAK1 were observed to be down-regulated. In addition, mRNA levels of HOXA1, a potent oncogene which has previously been implicated in autocrine hGH-mediated oncogenesis, was up-regulated 6-fold with over-expression of autocrine-hGH (FIG. 2d).

### Autocrine-hGH Expression by Endometrial Carcinoma Cells Enhances Anchorage-Independent Growth

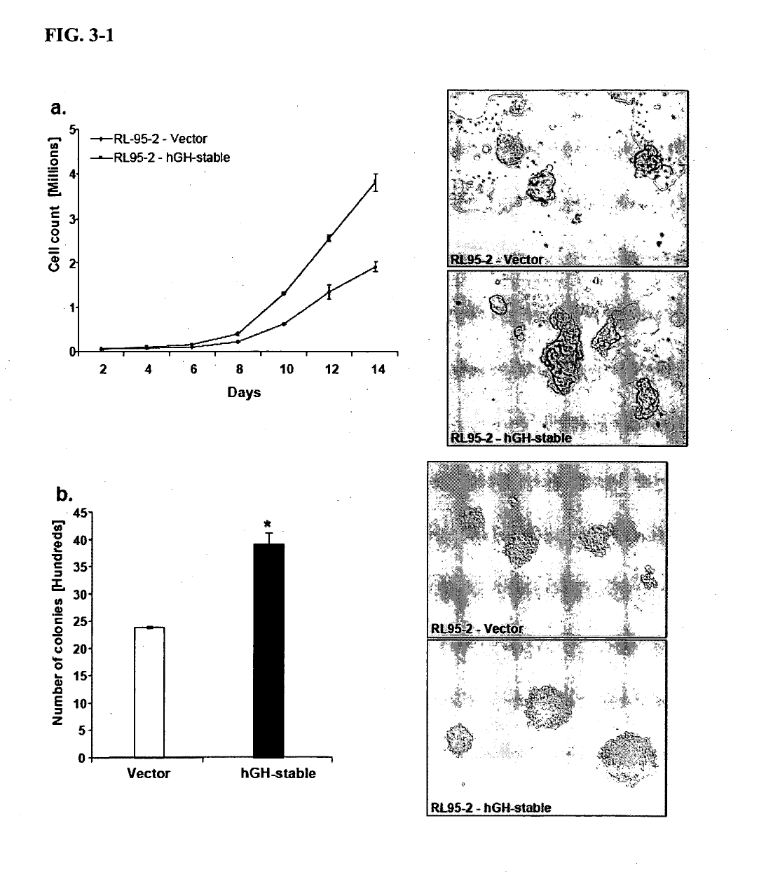

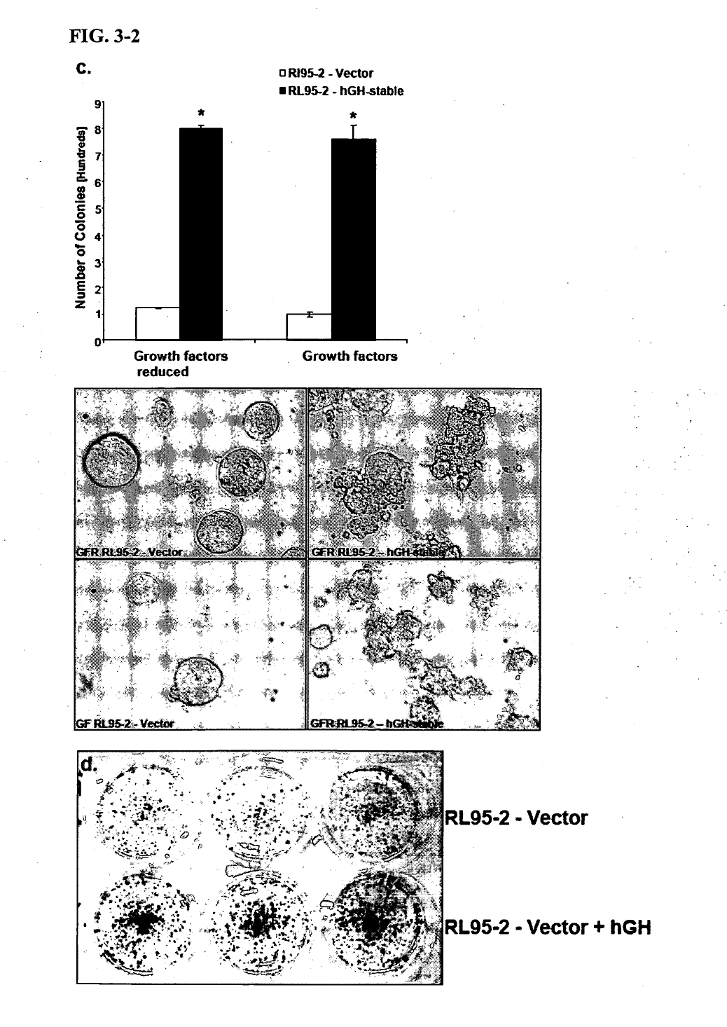

A characteristic of oncogenically transformed cells is anchorage-independent cell growth (Evan G, Littlewood T (1998). A matter of life and cell death. Science 281: 1317-22; Zhu Z, Mukhina S, Zhu T, Mertani H C, Lee K O, Lobie P E (2005). p44/42 MAP kinase-dependent regulation of catalase by autocrine human growth hormone protects human mammary carcinoma cells from oxidative stress-induced apoptosis. Oncogene 24: 3774-85; Hanahan D, Weinberg R A (2000). The hallmarks of cancer. Cell 100: 57-70). Autocrine expression of hGH in RL95-2-hGH cells enhanced anchorage-independent growth as indicated by colony formation in soft agar (FIG. 3a) and growth in suspension culture (FIG. 3b). Furthermore, the individual colony size in soft agar was markedly increased by autocrine-hGH (FIG. 3a). Higher expression of autocrine-hGH in RL95-2-hGH stable cells also increased foci formation when compared with the RL95-2-vector control cell line (FIG. 3d).

Oncogenically transformed epithelial cells form large, non-polarized, undifferentiated colonies without lumina when grown in Matrigel (Petersen O W, Ronnov-Jessen L, Howlett A R, Bissell M J (1992). Interaction with basement membrane serves to rapidly distinguish growth and differentiation pattern of normal and malignant human breast epithelial cells. Proc Natl Acad Sci USA 89: 9064-8). This ex-vivo model was used to examine the effects of autocrine hGH on the luminal architecture in Matrigel (Muthuswamy S K, Li D, Lelievre S, Bissell M J, Brugge J S (2001). ErbB2, but not ErbB1, reinitiates proliferation and induces luminal repopulation in epithelial acini. Nat Cell Biol 3: 785-92). RL95-2-vector cells and RL95-2-hGH cells were plated in growth factor-containing and growth factor-reduced Matrigel. Regular three-dimensional structures were generated by RL95-2-vector cells. In contrast, RL95-2-hGH cells formed disorganised structures of irregular morphology (FIG. 3c). In addition, RL95-2-hGH cells grown in growth factor-reduced Matrigel were more aggressive and displayed disrupted cellular polarization (FIG. 3c).

### Autocrine Expression of hGH in Stable Cells Promotes a Mesenchymal Phenotype

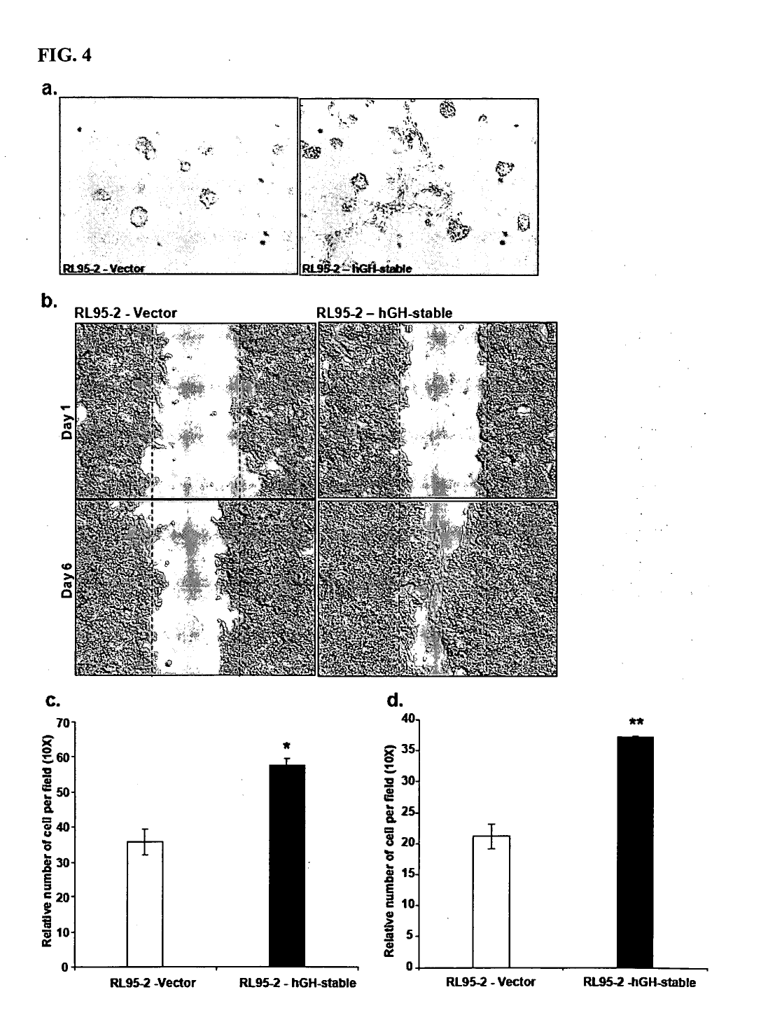

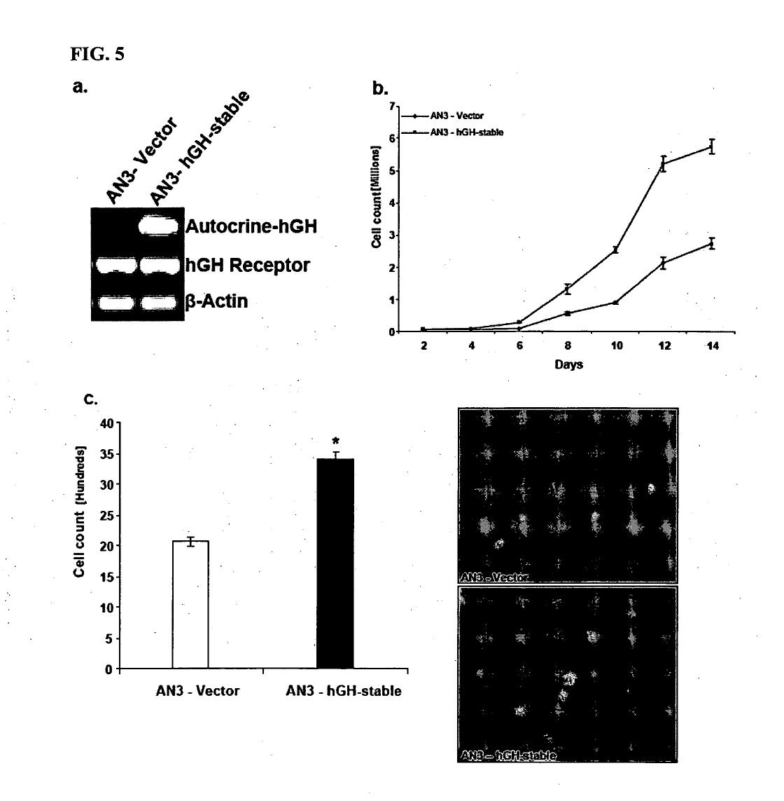

During the progression of carcinoma toward a less differentiated and more malignant state, cells lose their epithelial characteristics and acquire a mesenchymal morphology, together with concomitant changes in gene expression (Sommers C L, Byers S W, Thompson E W, Torri J A, Gelmann E P (1994). Differentiation state and invasiveness of human breast cancer cell lines. Breast Cancer Res Treat 31: 325-35; Thiery J P (2002). Epithelial-mesenchymal transitions in tumour progression. Nat Rev Cancer 2: 442-54). Such a phenotypic conversion is referred to as epithelial-mesenchymal transition. Autocrine-hGH in human mammary carcinoma cells has been demonstrated to alter cellular morphology and has been shown sufficient for generation of an invasive phenotype (Mukhina S, Mertani H C, Guo K, Lee K O, Gluckman P D, Lobie P E (2004). Phenotypic conversion of human mammary carcinoma cells by autocrine human growth hormone. Proc Natl Acad Sci USA 101: 15166-71). Similarly, it was found that expression of autocrine-hGH in RL95-2-hGH cells was associated with altered cell morphology. RL95-2-hGH cells also exhibited a mesenchymal phenotype when compared with control cells (FIG. 4a). Expression of fibronectin (FN1), an extracellular matrix protein and; alpha-catenin, beta-catenin, and delta-catenin, (catenin family) were also up-regulated with over-expression of autocrine-hGH.

The observed mesenchymal morphology of autocrine-hGH expressing cells was suggestive of an increased potential for migration and invasion. When cell motility was examined using a wound-healing assay, it was observed that autocrine-hGH did indeed stimulate cell migration with a more rapid closing of the wound than observed in vector transfected cells (FIG. 4b). Increased migration of autocrine-hGH expressing cells was also confirmed using a Transwell assay which compared the control cell line (FIG. 4c) with the RL95-2 parental cell line. Autocrine-hGH expressing RL95-2 cells also exhibited a significantly increased ability to pass through Matrigel in a standard invasion assay (FIG. 4d).

### Effect of Autocrine hGH Expression on AN3 Cell Line

In order to demonstrate that the effect of autocrine hGH on RL95-2 cells was not a cell type-specific phenomenon, the effects of autocrine expression of hGH in a second endometrial carcinoma cell line (AN3) were examined. AN3 cells overexpressing hGH, and a corresponding control cell line, were generated by stable transfection of AN3-hGH and AN3-vector as described for RL95-2 cells. RT-PCR and ELISA analysis demonstrated that AN3-hGH cells had increased hGH mRNA and protein levels when compared with the control cell line (FIG. 5a) and that both cell lines had equivalent levels of hGH-receptor transcript (FIG. 5a). Cell growth of the two stable cell lines was assessed in 10% serum medium. The total cell number of AN3-hGH cells was significantly increased over control cells during a 14 day time period, despite an originally identical plating density (FIG. 5b). Autocrine expression of hGH in stable cells also enhanced anchorage-independent cell growth and colony size was markedly increased as indicated by colony formation in soft agar (FIG. 5c).

hGH-antisera reduce the effects of endogenously expressed autocrine-hGH

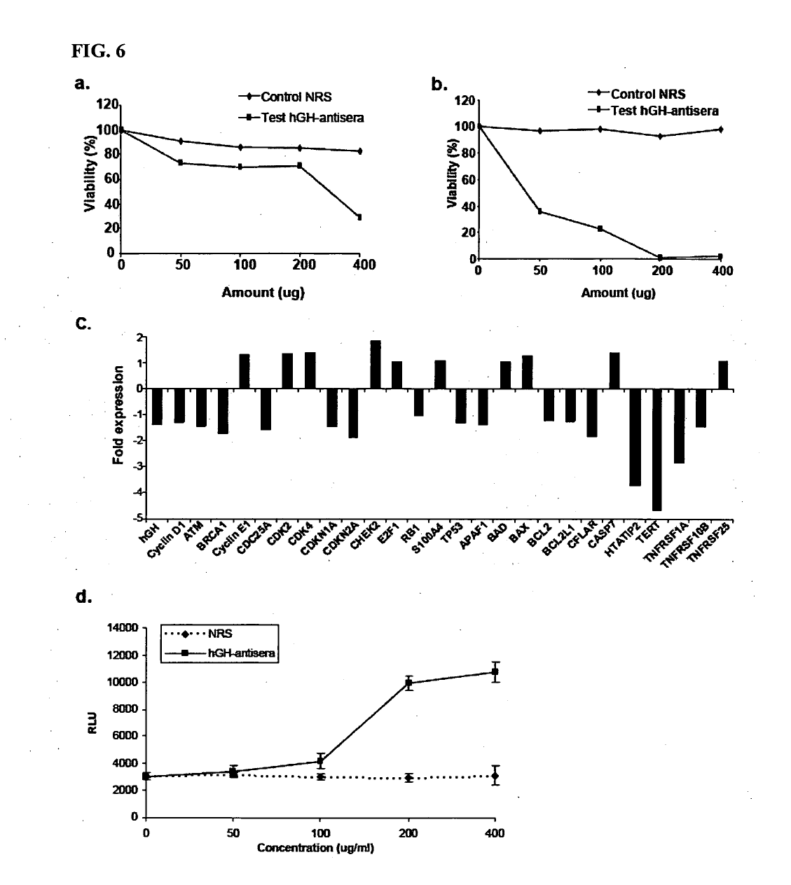

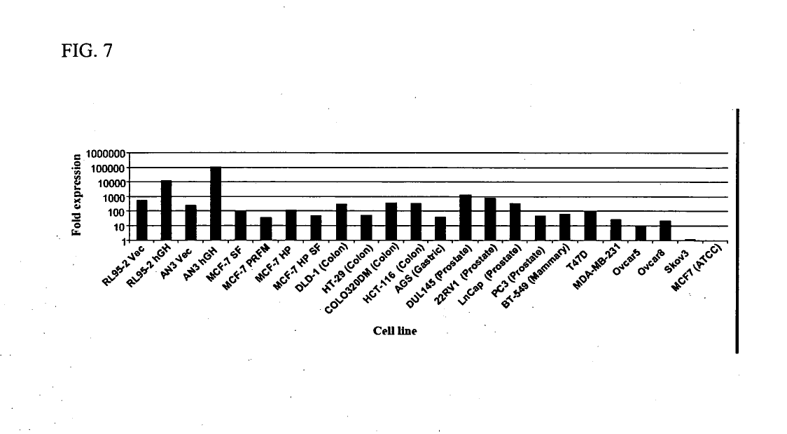

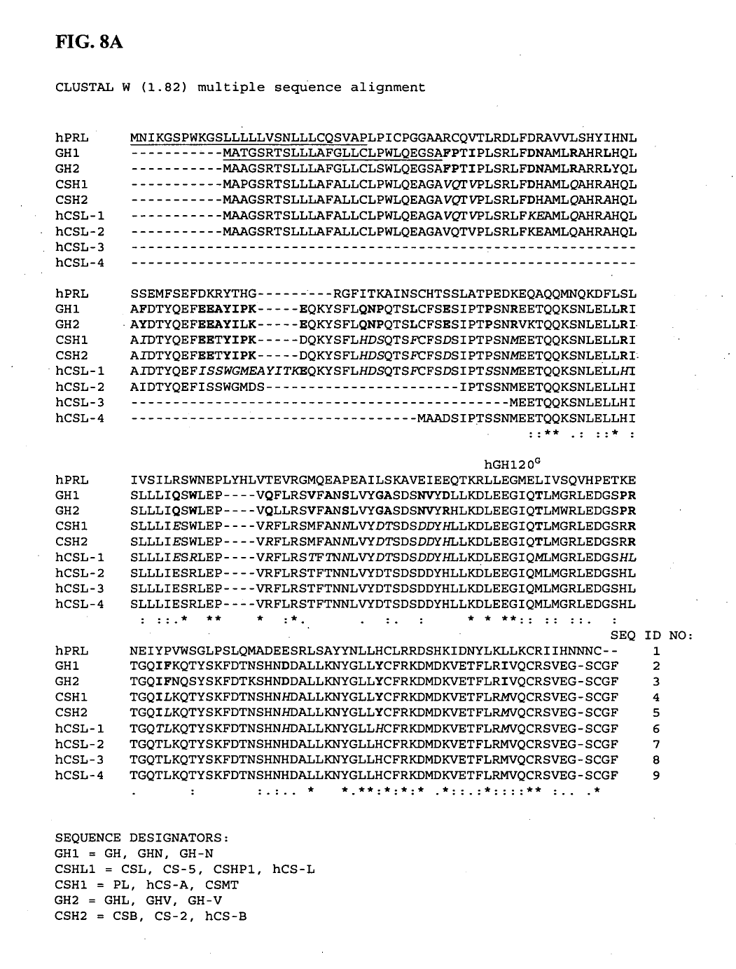

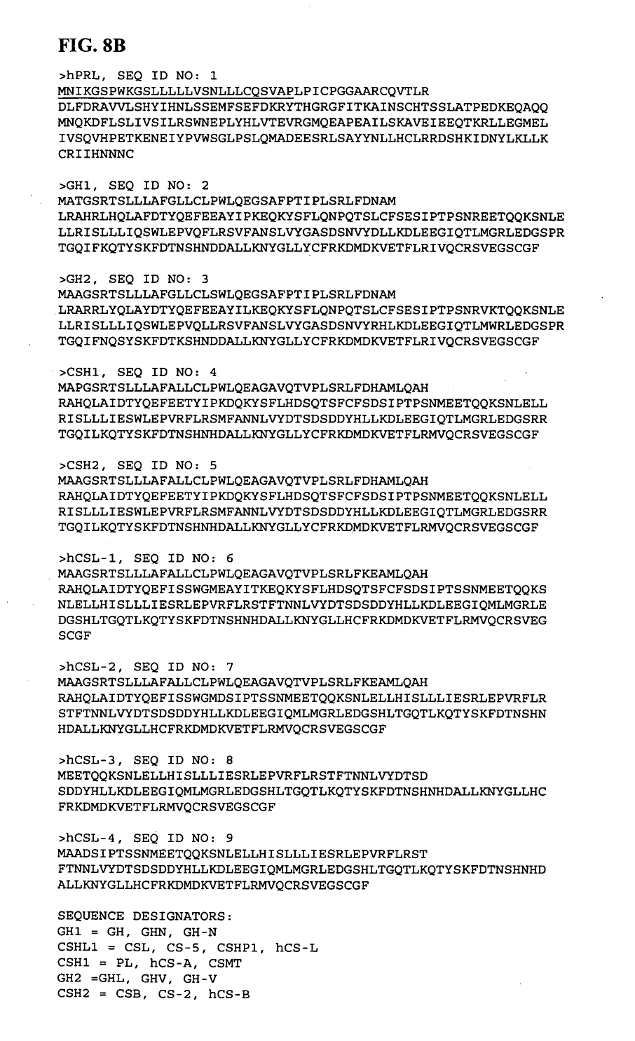

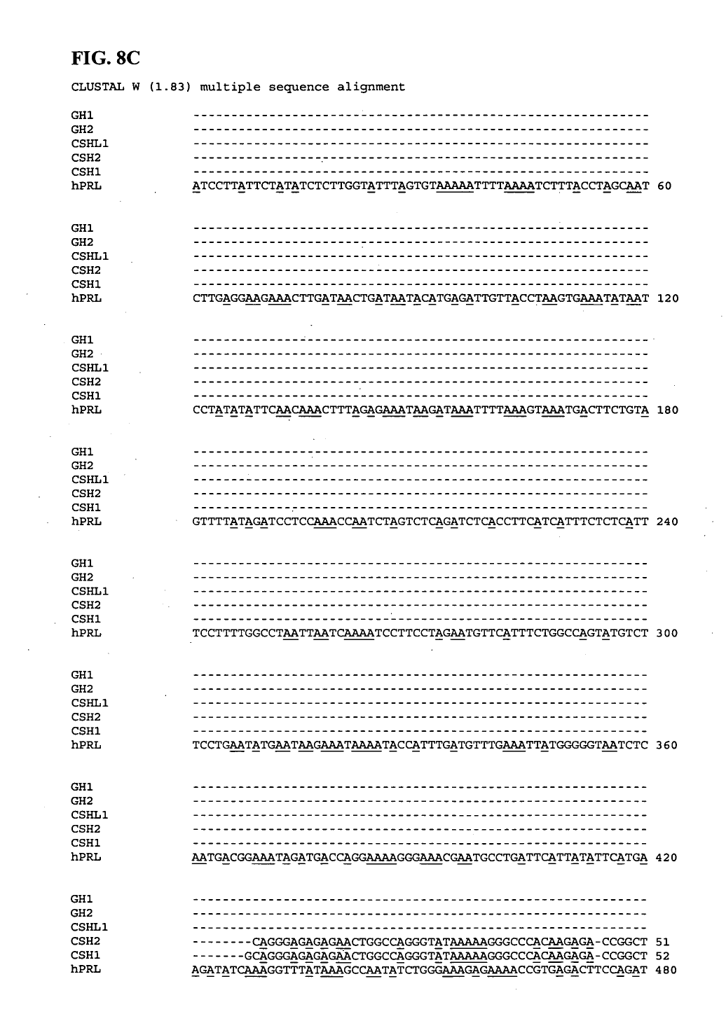

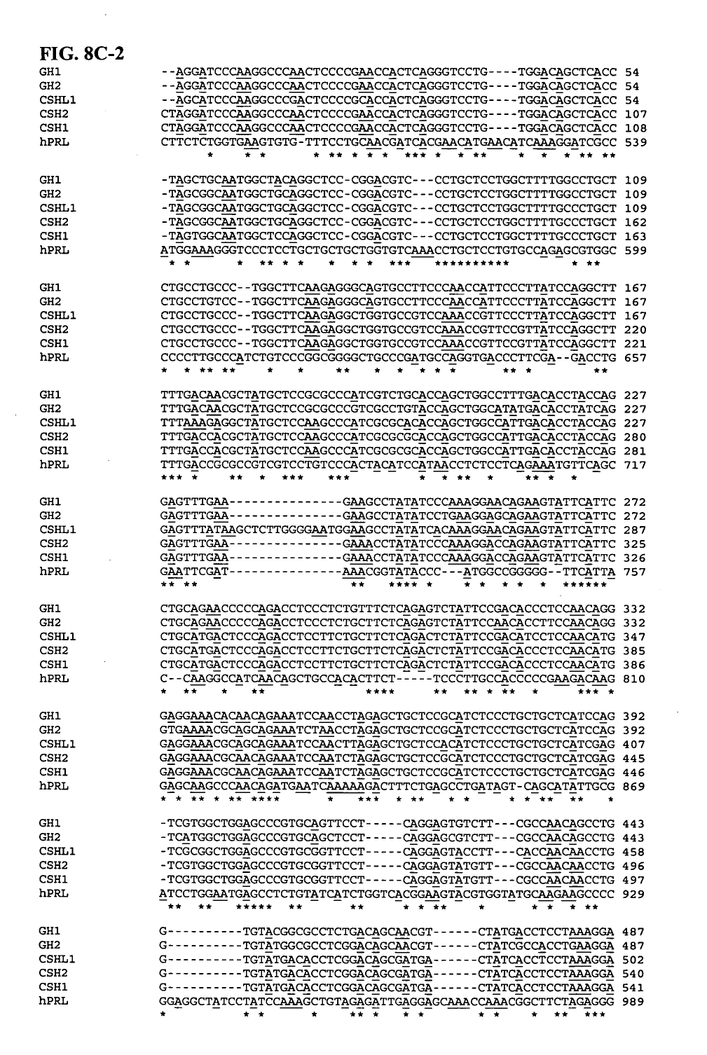

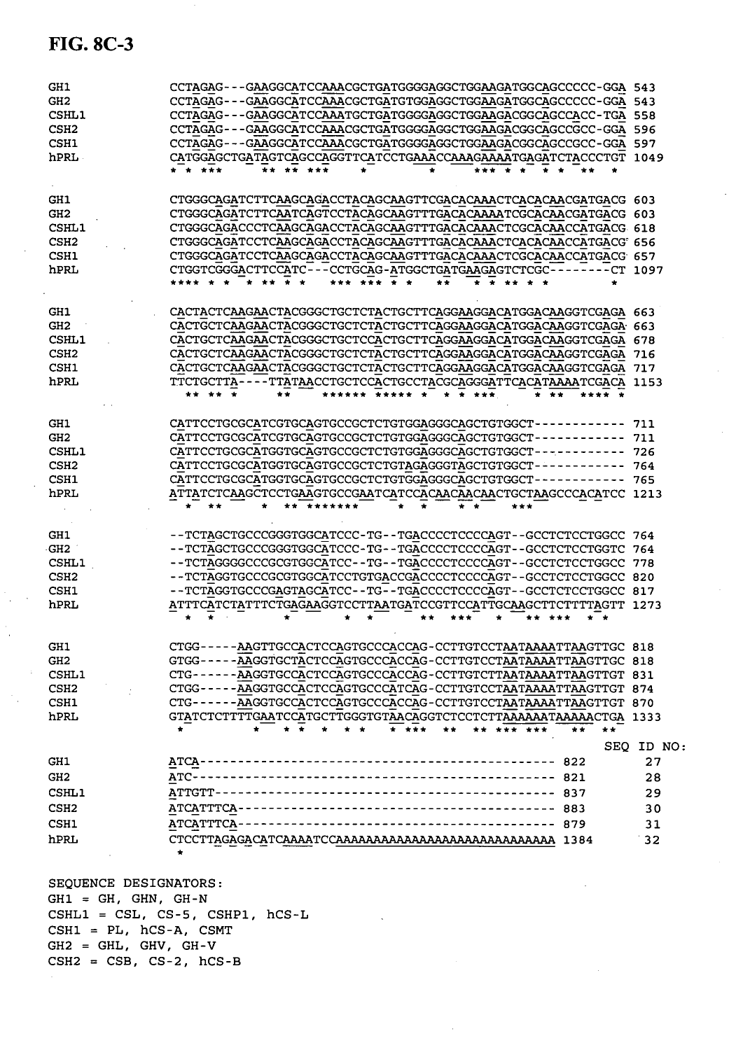

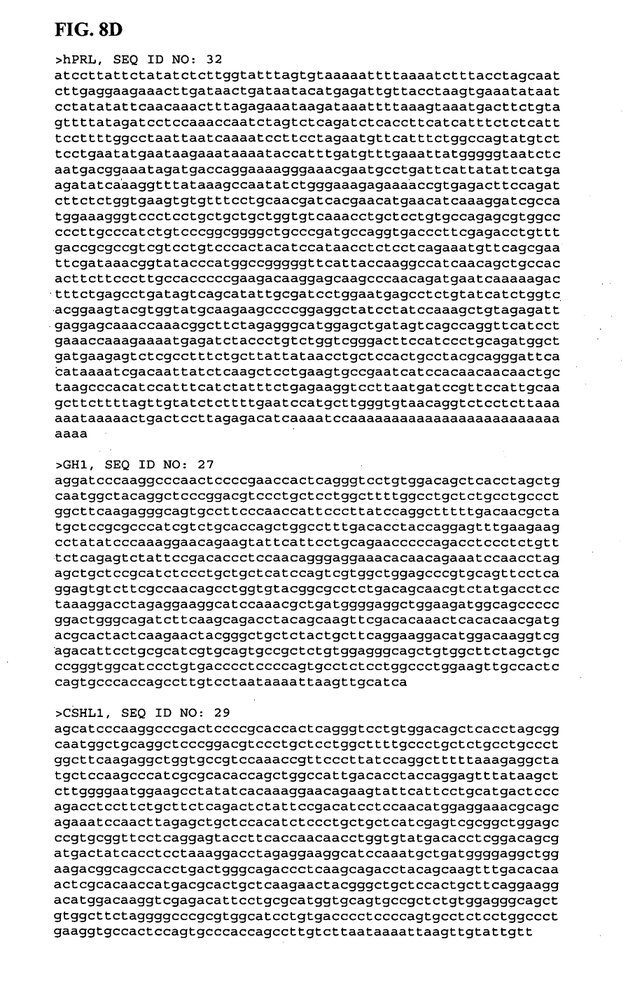

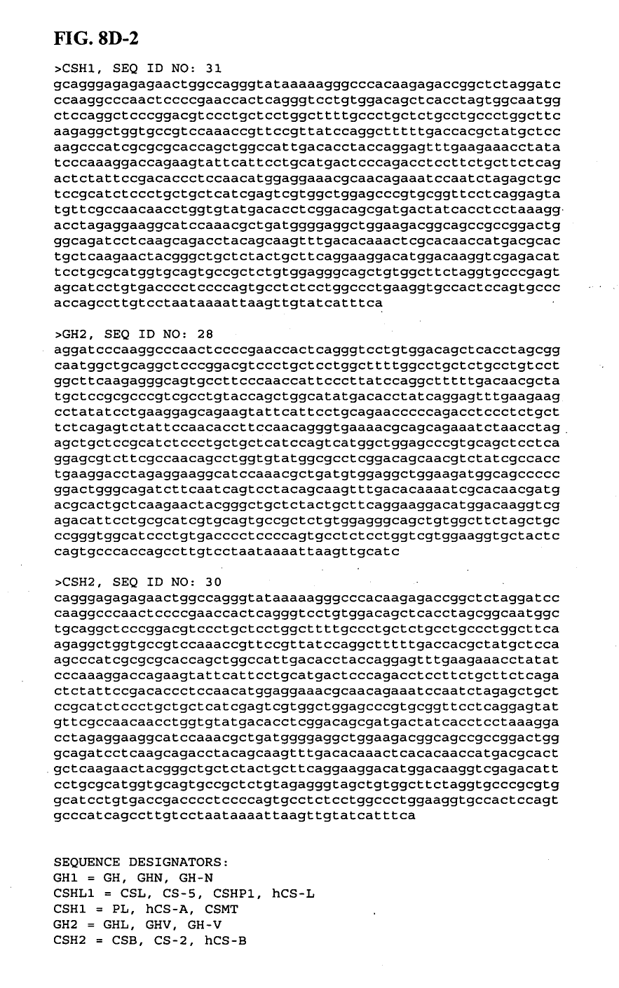

Wild-type RL95-2 cells produce a small amount of hGH protein. Therefore, the effects of antibodies to hGH on RL95-2 cellular function was investigated. hGH-antibodies, raised against hGH in rabbit were used. Treatment of RL95-2 cells with differing amounts of hGH-antibodies reduced cell viability over a 4 day time period both in 10% serum and serum-deficient medium as demonstrated using a MTT assay (FIG. 6 a & b). Similar results were also obtained for polyclonal chicken IgY raised against hGH. Expression analysis for various gene markers for cell cycle and apoptosis also demonstrated that treatment of cells with hGH antibodies resulted in an expression profile consistent with apoptosis. (FIG. 6c). The onset of apoptosis by antibodies to hGH was further confirmed by increased caspase 3/7 activity with increasing conentration of antibodies to hGH (FIG. 6d).

Depletion of hGH mRNA in Endometrial Carcinoma Cells Increases Apoptosis

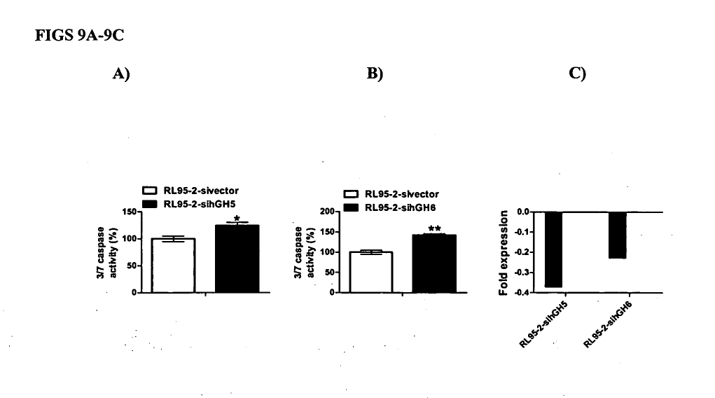

The effect of depletion of hGH mRNA on RL95-2 cellular function was examined using siRNA. Stable expression of two siRNA constructs (sihGH5 (SEQ ID NO: 97) and sihGH6 (SEQ ID NO: 98)) increased apoptotoic activity in RL95-2 cells, as measured by assaying 3/7 caspase activity (FIGS. 9A and 9B). Depletion of hGH mRNA in RL-95 cells by si hGH5 and sihGH6 was confirmed by q-PCR (FIG. 9C).

The invention has been described herein, with reference to certain preferred embodiments, in order to assist the reader in practising the invention without undue experimentation. However, a person having ordinary skill in the art will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all patent applications, patents, and publications, cited above and below, if any, are hereby incorporated by reference in their entirety.

Throughout this specification, and any claims which follow, unless the context requires otherwise, the words “comprise,” “comprising” and the like, are to be construed in an inclusive sense as opposed to an exclusive sense, that is to say, in the sense of “including, but not limited to”.

1. A method of inhibiting proliferation or survival of a tumor cell, comprising contacting said cell with an inhibitory agent of:
a) a gene member of the human growth hormone gene cluster, a proliferin gene or a prolactin gene; or
b) a peptide product of a gene member of the human growth hormone gene cluster, a proliferin gene or a prolactin gene.

2. A method of treating or preventing cancer, a cell proliferation disorder or a cell survival disorder in a subject in need thereof, comprising contacting said cell with an inhibitory agent of:
a) a gene member of the human growth hormone gene cluster, a proliferin gene or a prolactin gene; or
b) a peptide product of a gene member of the human growth hormone gene cluster, a proliferin gene or a prolactin gene.

3. The method of claim 1, wherein the gene member of the human growth hormone gene cluster is selected from the group consisting of human growth hormone 1 (hGH1) gene, human growth hormone 2 (hGH2) gene, chorionic somatomammotropin hormone 1 (CSH1) gene, chorionic somatomammotropin hormone 2 (CSH2) gene, chorionic somatomammotropin-like hormone (CSL) gene, chorionic somatomammotropin-like 2 hormone (CSL-2) gene, chorionic somatomammotropin-like 3 hormone (CSL-3) gene, and chorionic somatomammotropin-like 4 hormone (CSL-4) gene.

4. The method of claim 1, wherein the proliferin gene is a gene for proliferin or proliferin-related protein.

5. The method of claim 1, wherein the prolactin gene is a gene for prolactin or prolactin-related protein.

6. The method of claim 1, wherein said inhibitory agent is an isolated siRNA capable of inhibiting expression of a peptide product of a gene member of the human growth hormone gene cluster, a proliferin gene or a prolactin gene.

7. The method of claim 1, where said inhibitory agent is an isolated siRNA capable of inhibiting expression of a peptide product of a gene member of the human growth hormone gene cluster.

8. The method of claim 1, wherein said peptide product of a gene member of the human growth hormone gene cluster is selected from the group consisting of: prolactin (PRL), prolactin-related protein, growth hormone 1 (GH1), growth hormone 2 (GH2), chorionic somatomammotropin hormone 1 (CSH1), chorionic somatomammotropin hormone 2 (CSH2), chorionic somatomammotropin-like hormone (CSL), chorionic somatomammotropin-like 2 hormone (CSL-2), chorionic somatomammotropin-like 3 hormone (CSL-3), chorionic somatomammotropin-like 4 hormone (CSL-4), proliferin and proliferin-related protein.

9. The method of claim 1, wherein said peptide product of a proliferin gene is proliferin or proliferin-related protein.

10. The method of claim 1, wherein said peptide product of a prolactin gene is prolactin or prolactin-related protein.

11. The method of claim 7, wherein said isolated siRNA comprises a nucleotide sequence selected from the group consisting of SEQ ID NOS: 33-60 and 97-98.

12. The method of claim 1 wherein said inhibitory agent is an antibody that binds to a linear or conformational epitope on a peptide product of a gene member of the human growth hormone gene cluster, a proliferin gene or a prolactin gene.

13. The method of claim 12, wherein said inhibitory agent is an antibody that binds to a human growth hormone polypeptide or a human prolactin polypeptide.

14. The method of claim 13, wherein said human growth hormone polypeptide is selected from the group consisting of SEQ ID NOS: 10-20.

15. The method of claim 13 wherein said human prolactin polypeptide is selected from the group consisting of SEQ ID NOS: 21-26.

16. The method of claim 1, wherein said tumor cell is an epithelial tumor cell.

17. The method of claim 16, wherein said epithelial tumor cell is from a tumor selected from lung cancer, colon cancer, breast cancer, prostate cancer, and endometrial carcinoma.

18. The method of claim 2, wherein said cell proliferation disorder is endometriosis.

19. The method of claim 1, further comprising the administration of a second compound wherein said second compound is a chemotherapeutic or anti-neoplastic agent.

20. An antibody that specifically binds a human growth hormone polypeptide, wherein said antibody (a) binds to a conformational epitope on said human growth hormone polypeptide; (b) binds to a sequential epitope on said human growth hormone polypeptide; or (c) comprises an antigenic determinant selected from the antigenic determinants shown in Table 1.

21. The antibody of claim 20, wherein said conformational epitope is selected from a conformational epitope shown in Table 2.

22. (canceled)

23. The antibody of claim 20, wherein said sequential epitope is selected from a sequential epitope shown in Table 3.

24. (canceled)

25. An antibody that specifically binds a human prolactin polypeptide, wherein said antibody (a) binds to a conformational epitope on said human prolactin polypeptide; or (b) comprises an antigenic determinant selected from the antigenic determinants shown in Table 4.

26. The antibody of claim 25, wherein said conformational epitope is selected from a conformational epitope shown in Table 5.

27. (canceled)

28. A composition comprising an antibody according to claim 20, wherein said composition further comprises a pharmaceutically acceptable carrier.

29. An isolated siRNA comprising (a) a nucleotide sequence selected from the group consisting of SEQ ID NO: 33-39 and 97-98, wherein said isolated siRNA is capable of inhibiting expression of a human growth hormone polypeptide; or (b) a sense RNA strand and an antisense RNA strand, wherein the sense and antisense RNA strands form an RNA duplex, wherein said isolated siRNA comprises a nucleotide sequence selected from the group consisting of SEQ ID NOS: 40-60, and wherein said siRNA is capable of inhibiting expression of a human growth hormone polypeptide.

30. (canceled)

31. The siRNA of claim 29, wherein said human growth hormone polypeptide is selected from the group consisting of: prolactin (PRL), prolactin-related protein, growth hormone 1 (GH1), growth hormone 2 (GH2), chorionic somatomammotropin hormone 1 (CSH1), chorionic somatomammotropin hormone 2 (CSH2), chorionic somatomammotropin-like hormone (CSL), chorionic somatomammotropin-like 2 hormone (CSL-2), chorionic somatomammotropin-like 3 hormone (CSL-3), (chorionic somatomammotropin-like 4 hormone (CSL-4), proliferin and proliferin-related protein.

32. A composition comprising an siRNA according to claim 29, wherein said composition further comprises a pharmaceutically acceptable carrier.

33. A method of diagnosing a cancer, a cell proliferation disorder or a cell survival disorder in a subject, comprising contacting a test sample from said subject with an antibody according to claim 20 and detecting the level of antibody that binds to said sample, wherein an increase in the level of antibody binding in said sample compared to the level of binding in a control sample indicates the presence of a cancer, a cell proliferation disorder or a cell survival disorder.

34. The method of claim 33, wherein said cancer is an epithelial cancer.

35. The method of claim 34, wherein said epithelial cancer is selected from lung cancer, colon cancer, breast cancer, prostate cancer, endometrial carcinoma.

36. The method of claim 33, wherein said cell proliferation disorder is endometriosis.

37. The method of claim 1, wherein said subject is a human subject.


SEQUENCE LISTING (98 sequences)

SEQ ID NO: 1 - protein, 227 aa, Homo sapiens
MNIKGSPWKGSLLLLLVSNLLLCQSVAPLPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 2 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 3 - protein, 217 aa, Homo sapiens
MAAGSRTSLLLAFGLLCLSWLQEGSAFPTIPLSRLFDNAMLRARRLYQLAYDTYQEFEEAYILKEQKYSFLQNPQTSLCFSESIPTPSNRVKTQQKSNLELLRISLLLIQSWLEPVQLLRSVFANSLVYGASDSNVYRHLKDLEEGIQTLMWRLEDGSPRTGQIFNQSYSKFDTKSHNDDALLKNYGLLYCFRKDMDKVETFLRIVQCRSVEGSCGF

SEQ ID NO: 4 - protein, 217 aa, Homo sapiens
MAPGSRTSLLLAFALLCLPWLQEAGAVQTVPLSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEEGIQTLMGRLEDGSRRTGQILKQTYSKFDTNSHNHDALLKNYGLLYCFRKDMDKVETFLRMVQCRSVEGSCGF

SEQ ID NO: 5 - protein, 217 aa, Homo sapiens
MAAGSRTSLLLAFALLCLPWLQEAGAVQTVPLSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEEGIQTLMGRLEDGSRRTGQILKQTYSKFDTNSHNHDALLKNYGLLYCFRKDMDKVETFLRMVQCRSVEGSCGF

SEQ ID NO: 6 - protein, 222 aa, Homo sapiens
MAAGSRTSLLLAFALLCLPWLQEAGAVQTVPLSRLFKEAMLQAHRAHQLAIDTYQEFISSWGMEAYITKEQKYSFLHDSQTSFCFSDSIPTSSNMEETQQKSNLELLHISLLLIESRLEPVRFLRSTFTNNLVYDTSDSDDYHLLKDLEEGIQMLMGRLEDGSHLTGQTLKQTYSKFDTNSHNHDALLKNYGLLHCFRKDMDKVETFLRMVQCRSVEGSCGF

SEQ ID NO: 7 - protein, 199 aa, Homo sapiens
MAAGSRTSLLLAFALLCLPWLQEAGAVQTVPLSRLFKEAMLQAHRAHQLAIDTYQEFISSWGMDSIPTSSNMEETQQKSNLELLHISLLLIESRLEPVRFLRSTFTNNLVYDTSDSDDYHLLKDLEEGIQMLMGRLEDGSHLTGQTLKQTYSKFDTNSHNHDALLKNYGLLHCFRKDMDKVETFLRMVQCRSVEGSCGF

SEQ ID NO: 8 - protein, 128 aa, Homo sapiens
MEETQQKSNLELLHISLLLIESRLEPVRFLRSTFTNNLVYDTSDSDDYHLLKDLEEGIQMLMGRLEDGSHLTGQTLKQTYSKFDTNSHNHDALLKNYGLLHCFRKDMDKVETFLRMVQCRSVEGSCGF

SEQ ID NO: 9 - protein, 139 aa, Homo sapiens
MAADSIPTSSNMEETQQKSNLELLHISLLLIESRLEPVRFLRSTFTNNLVYDTSDSDDYHLLKDLEEGIQMLMGRLEDGSHLTGQTLKQTYSKFDTNSHNHDALLKNYGLLHCFRKDMDKVETFLRMVQCRSVEGSCGF

SEQ ID NO: 10 - protein, 11 aa, Artificial Sequence
  predicted hGH antigenic determinant
PTIPLSRLFQN

SEQ ID NO: 11 - protein, 3 aa, Artificial Sequence
  predicted hGH antigenic determinant
EAY

SEQ ID NO: 12 - protein, 13 aa, Artificial Sequence
  predicted hGH antigenic determinant
QKYSFIQAPQASL

SEQ ID NO: 13 - protein, 19 aa, Artificial Sequence
  predicted hGH antigenic determinant
ESIPTPSNREQAQQKSNIQ

SEQ ID NO: 14 - protein, 22 aa, Artificial Sequence
  predicted hGH antigenic determinant
GFLRSVFANSLVYGASDSDVYD

SEQ ID NO: 15 - protein, 29 aa, Artificial Sequence
  predicted hGH antigenic determinant
GRLEDGSPRTGQAFKQTYAKFDANSHNDD

SEQ ID NO: 16 - protein, 9 aa, Artificial Sequence
  predicted hGH antigenic determinant
CRSVEGSCG

SEQ ID NO: 17 - protein, 3 aa, Artificial Sequence
  predicted hGH conformational epitope
PKE

SEQ ID NO: 18 - protein, 7 aa, Artificial Sequence
  predicted hGH sequential epitope
EAYIPKE

SEQ ID NO: 19 - protein, 16 aa, Artificial Sequence
  predicted hGH sequential epitope
PKEQKYSFIQAPQASL

SEQ ID NO: 20 - protein, 20 aa, Artificial Sequence
  predicted hGH sequential epitope
EAYIPKEQKYSFIQAPQASL

SEQ ID NO: 21 - protein, 13 aa, Artificial Sequence
  predicted hPRL antigenic determinant
LPICPGGAARCQV

SEQ ID NO: 22 - protein, 28 aa, Artificial Sequence
  predicted hPRL antigenic determinant
YTHGRGFITKAINSCHTSSLATPEDKEQ

SEQ ID NO: 23 - protein, 4 aa, Artificial Sequence
  predicted hPRL antigenic determinant
QMNQ

SEQ ID NO: 24 - protein, 12 aa, Artificial Sequence
  predicted hPRL antigenic determinant
RGMQEAPEAILS

SEQ ID NO: 25 - protein, 30 aa, Artificial Sequence
  predicted hPRL antigenic determinant
SQVHPETKENEIYPVWSGLPSIQMADEESR

SEQ ID NO: 26 - protein, 13 aa, Artificial Sequence
  predicted hPRL antigenic determinant
KLLKCRIIHNNNC

SEQ ID NO: 27 - DNA, 822 bp, homon sapiens
aggatcccaaggcccaactccccgaaccactcagggtcctgtggacagctcacctagctgcaatggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatca

SEQ ID NO: 28 - DNA, 821 bp, homo sapiens
aggatcccaaggcccaactccccgaaccactcagggtcctgtggacagctcacctagcggcaatggctgcaggctcccggacgtccctgctcctggcttttggcctgctctgcctgtcctggcttcaagagggcagtgccttcccaaccattcccttatccaggctttttgacaacgctatgctccgcgcccgtcgcctgtaccagctggcatatgacacctatcaggagtttgaagaagcctatatcctgaaggagcagaagtattcattcctgcagaacccccagacctccctctgcttctcagagtctattccaacaccttccaacagggtgaaaacgcagcagaaatctaacctagagctgctccgcatctccctgctgctcatccagtcatggctggagcccgtgcagctcctcaggagcgtcttcgccaacagcctggtgtatggcgcctcggacagcaacgtctatcgccacctgaaggacctagaggaaggcatccaaacgctgatgtggaggctggaagatggcagcccccggactgggcagatcttcaatcagtcctacagcaagtttgacacaaaatcgcacaacgatgacgcactgctcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggtcgtggaaggtgctactccagtgcccaccagccttgtcctaataaaattaagttgcatc

SEQ ID NO: 29 - DNA, 837 bp, homo sapiens
agcatcccaaggcccgactccccgcaccactcagggtcctgtggacagctcacctagcggcaatggctgcaggctcccggacgtccctgctcctggcttttgccctgctctgcctgccctggcttcaagaggctggtgccgtccaaaccgttcccttatccaggctttttaaagaggctatgctccaagcccatcgcgcacaccagctggccattgacacctaccaggagtttataagctcttggggaatggaagcctatatcacaaaggaacagaagtattcattcctgcatgactcccagacctccttctgcttctcagactctattccgacatcctccaacatggaggaaacgcagcagaaatccaacttagagctgctccacatctccctgctgctcatcgagtcgcggctggagcccgtgcggttcctcaggagtaccttcaccaacaacctggtgtatgacacctcggacagcgatgactatcacctcctaaaggacctagaggaaggcatccaaatgctgatggggaggctggaagacggcagccacctgactgggcagaccctcaagcagacctacagcaagtttgacacaaactcgcacaaccatgacgcactgctcaagaactacgggctgctccactgcttcaggaaggacatggacaaggtcgagacattcctgcgcatggtgcagtgccgctctgtggagggcagctgtggcttctaggggcccgcgtggcatcctgtgacccctccccagtgcctctcctggccctgaaggtgccactccagtgcccaccagccttgtcttaataaaattaagttgtattgtt

SEQ ID NO: 30 - DNA, 883 bp, homo sapiens
cagggagagagaactggccagggtataaaaagggcccacaagagaccggctctaggatcccaaggcccaactccccgaaccactcagggtcctgtggacagctcacctagcggcaatggctgcaggctcccggacgtccctgctcctggcttttgccctgctctgcctgccctggcttcaagaggctggtgccgtccaaaccgttccgttatccaggctttttgaccacgctatgctccaagcccatcgcgcgcaccagctggccattgacacctaccaggagtttgaagaaacctatatcccaaaggaccagaagtattcattcctgcatgactcccagacctccttctgcttctcagactctattccgacaccctccaacatggaggaaacgcaacagaaatccaatctagagctgctccgcatctccctgctgctcatcgagtcgtggctggagcccgtgcggttcctcaggagtatgttcgccaacaacctggtgtatgacacctcggacagcgatgactatcacctcctaaaggacctagaggaaggcatccaaacgctgatggggaggctggaagacggcagccgccggactgggcagatcctcaagcagacctacagcaagtttgacacaaactcacacaaccatgacgcactgctcaagaactacgggctgctctactgcttcaggaaggacatggacaaggtcgagacattcctgcgcatggtgcagtgccgctctgtagagggtagctgtggcttctaggtgcccgcgtggcatcctgtgaccgacccctccccagtgcctctcctggccctggaaggtgccactccagtgcccatcagccttgtcctaataaaattaagttgtatcatttca

SEQ ID NO: 31 - DNA, 879 bp, homo sapiens
gcagggagagagaactggccagggtataaaaagggcccacaagagaccggctctaggatcccaaggcccaactccccgaaccactcagggtcctgtggacagctcacctagtggcaatggctccaggctcccggacgtccctgctcctggcttttgccctgctctgcctgccctggcttcaagaggctggtgccgtccaaaccgttccgttatccaggctttttgaccacgctatgctccaagcccatcgcgcgcaccagctggccattgacacctaccaggagtttgaagaaacctatatcccaaaggaccagaagtattcattcctgcatgactcccagacctccttctgcttctcagactctattccgacaccctccaacatggaggaaacgcaacagaaatccaatctagagctgctccgcatctccctgctgctcatcgagtcgtggctggagcccgtgcggttcctcaggagtatgttcgccaacaacctggtgtatgacacctcggacagcgatgactatcacctcctaaaggacctagaggaaggcatccaaacgctgatggggaggctggaagacggcagccgccggactgggcagatcctcaagcagacctacagcaagtttgacacaaactcgcacaaccatgacgcactgctcaagaactacgggctgctctactgcttcaggaaggacatggacaaggtcgagacattcctgcgcatggtgcagtgccgctctgtggagggcagctgtggcttctaggtgcccgagtagcatcctgtgacccctccccagtgcctctcctggccctgaaggtgccactccagtgcccaccagccttgtcctaataaaattaagttgtatcatttca

SEQ ID NO: 32 - DNA, 1384 bp, homo sapiens
atccttattctatatctcttggtatttagtgtaaaaattttaaaatctttacctagcaatcttgaggaagaaacttgataactgataatacatgagattgttacctaagtgaaatataatcctatatattcaacaaactttagagaaataagataaattttaaagtaaatgacttctgtagttttatagatcctccaaaccaatctagtctcagatctcaccttcatcatttctctcatttccttttggcctaattaatcaaaatccttcctagaatgttcatttctggccagtatgtcttcctgaatatgaataagaaataaaataccatttgatgtttgaaattatgggggtaatctcaatgacggaaatagatgaccaggaaaagggaaacgaatgcctgattcattatattcatgaagatatcaaaggtttataaagccaatatctgggaaagagaaaaccgtgagacttccagatcttctctggtgaagtgtgtttcctgcaacgatcacgaacatgaacatcaaaggatcgccatggaaagggtccctcctgctgctgctggtgtcaaacctgctcctgtgccagagcgtggcccccttgcccatctgtcccggcggggctgcccgatgccaggtgacccttcgagacctgtttgaccgcgccgtcgtcctgtcccactacatccataacctctcctcagaaatgttcagcgaattcgataaacggtatacccatggccgggggttcattaccaaggccatcaacagctgccacacttcttcccttgccacccccgaagacaaggagcaagcccaacagatgaatcaaaaagactttctgagcctgatagtcagcatattgcgatcctggaatgagcctctgtatcatctggtcacggaagtacgtggtatgcaagaagccccggaggctatcctatccaaagctgtagagattgaggagcaaaccaaacggcttctagagggcatggagctgatagtcagccaggttcatcctgaaaccaaagaaaatgagatctaccctgtctggtcgggacttccatccctgcagatggctgatgaagagtctcgcctttctgcttattataacctgctccactgcctacgcagggattcacataaaatcgacaattatctcaagctcctgaagtgccgaatcatccacaacaacaactgctaagcccacatccatttcatctatttctgagaaggtccttaatgatccgttccattgcaagcttcttttagttgtatctcttttgaatccatgcttgggtgtaacaggtctcctcttaaaaaataaaaactgactccttagagacatcaaaatccaaaaaaaaaaaaaaaaaaaaaaaaaaaaa

SEQ ID NO: 33 - DNA, 19 bp, Artificial Sequence
  synthetic siRNA
gagggcagtgccttcccaa

SEQ ID NO: 34 - DNA, 19 bp, Artificial Sequence
  synthetic siRNA
gcctatatcccaaaggaac

SEQ ID NO: 35 - DNA, 19 bp, Artificial Sequence
  synthetic siRNA
cctggtgtacggcgcctct

SEQ ID NO: 36 - DNA, 19 bp, Artificial Sequence
  synthetic siRNA
ctgacagcaacgtctatga

SEQ ID NO: 37 - DNA, 19 bp, Artificial Sequence
  synthetic siRNA
gtattcattcctgcagaac

SEQ ID NO: 38 - DNA, 19 bp, Artificial Sequence
  synthetic siRNA
cagcctggtgtacggcgcc

SEQ ID NO: 39 - DNA, 19 bp, Artificial Sequence
  synthetic siRNA
cgatgacgcactactcaag

SEQ ID NO: 40 - DNA, 47 bp, Artificial Sequence
  synthetic siRNA
gagggcagtgccttcccaattcaagagattgggaaggcactgccctc

SEQ ID NO: 41 - DNA, 47 bp, Artificial Sequence
  synthetic siRNA
gcctatatcccaaaggaacttcaagagagttcctttgggatataggc

SEQ ID NO: 42 - DNA, 46 bp, Artificial Sequence
  synthetic siRNA
cctggtgtacggcgcctctttcaagagaagaggcgccgtacaccag

SEQ ID NO: 43 - DNA, 47 bp, Artificial Sequence
  synthetic siRNA
ctgacagcaacgtctatgattcaagagatcatagacgttgctgtcag

SEQ ID NO: 44 - DNA, 47 bp, Artificial Sequence
  synthetic siRNA
gtattcattcctgcagaacttcaagagagttctgcaggaatgaatac

SEQ ID NO: 45 - DNA, 47 bp, Artificial Sequence
  synthetic siRNA
cagcctggtgtacggcgccttcaagagaggcgccgtacaccaggctg

SEQ ID NO: 46 - DNA, 47 bp, Artificial Sequence
  synthetic siRNA
cgatgacgcactactcaagttcaagagacttgagtagtgcgtcatcg

SEQ ID NO: 47 - DNA, 55 bp, Artificial Sequence
  synthetic siRNA
nnnngagggcagtgccttcccaattcaagagattgggaaggcactgccctcnnnn

SEQ ID NO: 48 - DNA, 55 bp, Artificial Sequence
  synthetic siRNA
nnnngcctatatcccaaaggaacttcaagagagttcctttgggatataggcnnnn

SEQ ID NO: 49 - DNA, 55 bp, Artificial Sequence
  synthetic siRNA
nnnngcctggtgtacggcgcctctttcaagagaagaggcgccgtacaccagnnnn

SEQ ID NO: 50 - DNA, 56 bp, Artificial Sequence
  synthetic siRNA
nnnngctgacagcaacgtctatgattcaagagatcatagacgttgctgtcagnnnn

SEQ ID NO: 51 - DNA, 55 bp, Artificial Sequence
  synthetic siRNA
nnnngtattcattcctgcagaacttcaagagagttctgcaggaatgaatacnnnn

SEQ ID NO: 52 - DNA, 56 bp, Artificial Sequence
  synthetic siRNA
nnnngcagcctggtgtacggcgccttcaagagaggcgccgtacaccaggctgnnnn

SEQ ID NO: 53 - DNA, 56 bp, Artificial Sequence
  synthetic siRNA
nnnngcgatgacgcactactcaagttcaagagacttgagtagtgcgtcatcgnnnn

SEQ ID NO: 54 - DNA, 69 bp, Artificial Sequence
  synthetic siRNA
ggatccgagggcagtgccttcccaattcaagagattgggaaggcactgccctcttttttggaaaagtcc

SEQ ID NO: 55 - DNA, 69 bp, Artificial Sequence
  synthetic siRNA
ggatccgcctatatcccaaaggaacttcaagagagttcctttgggatataggcttttttggaaaagtcc

SEQ ID NO: 56 - DNA, 71 bp, Artificial Sequence
  synthetic siRNA
ggatccgcctggtgtacggcgcctctttcaagagaagaggcgccgtacaccaggcttttttggaaaagtcc

SEQ ID NO: 57 - DNA, 72 bp, Artificial Sequence
  synthetic siRNA
ggatccgctgacagcaacgtctatgattcaagagatcatagacgttgctgtcagagttttttggaaaagtcc

SEQ ID NO: 58 - DNA, 69 bp, Artificial Sequence
  synthetic siRNA
ggatccgtattcattcctgcagaacttcaagagagttctgcaggaatgaatacttttttggaaaagtcc

SEQ ID NO: 59 - DNA, 70 bp, Artificial Sequence
  synthetic siRNA
ggatccgcagcctggtgtacggcgccttcaagagaggcgccgtacaccaggctgttttttggaaaagtcc

SEQ ID NO: 60 - DNA, 70 bp, Artificial Sequence
  synthetic siRNA
ggatccgcgatgacgcactactcaagttcaagagacttgagtagtgcgtcatcgttttttggaaaagtcc

SEQ ID NO: 61 - DNA, 20 bp, Artificial Sequence
  synthetic primer
ccgacaccctccaacaggga

SEQ ID NO: 62 - DNA, 20 bp, Artificial Sequence
  synthetic primer
ccttgtccatgtccttcctg

SEQ ID NO: 63 - DNA, 22 bp, Artificial Sequence
  synthetic primer
ctcaactggactttactgaacg

SEQ ID NO: 64 - DNA, 22 bp, Artificial Sequence
  synthetic primer
aatctttggaactggaactggg

SEQ ID NO: 65 - DNA, 18 bp, Artificial Sequence
  synthetic primer
atgatatcgccgcgctcg

SEQ ID NO: 66 - DNA, 19 bp, Artificial Sequence
  synthetic primer
cgctcggtgaggatcttca

SEQ ID NO: 67 - DNA, 17 bp, Artificial Sequence
  synthetic primer
ttcctgggcatggagtc

SEQ ID NO: 68 - DNA, 18 bp, Artificial Sequence
  synthetic primer
caggtctttgcggatgtc

SEQ ID NO: 69 - DNA, 21 bp, Artificial Sequence
  synthetic primer
tgacactggcaaaacaatgca

SEQ ID NO: 70 - DNA, 21 bp, Artificial Sequence
  synthetic primer
ggtccttttcaccagcaagct

SEQ ID NO: 71 - DNA, 20 bp, Artificial Sequence
  synthetic primer
tgcaccaccaactgcttagc

SEQ ID NO: 72 - DNA, 21 bp, Artificial Sequence
  synthetic primer
ggcatggactgtggtcatgag

SEQ ID NO: 73 - DNA, 20 bp, Artificial Sequence
  synthetic primer
cccatcagcaggaacacctt

SEQ ID NO: 74 - DNA, 22 bp, Artificial Sequence
  synthetic primer
ggctcactgcaaagactttgaa

SEQ ID NO: 75 - DNA, 22 bp, Artificial Sequence
  synthetic primer
ccatgcaggcaacataaacttc

SEQ ID NO: 76 - DNA, 23 bp, Artificial Sequence
  synthetic primer
agggttgtaacctgtgtaacaag

SEQ ID NO: 77 - DNA, 20 bp, Artificial Sequence
  synthetic primer
cctatgcaggggtggtcaac

SEQ ID NO: 78 - DNA, 20 bp, Artificial Sequence
  synthetic primer
cgacctggaaaacgccatca

SEQ ID NO: 79 - DNA, 19 bp, Artificial Sequence
  synthetic primer
cccaccacgtacaagggtc

SEQ ID NO: 80 - DNA, 19 bp, Artificial Sequence
  synthetic primer
ctggggtattgggggcatc

SEQ ID NO: 81 - DNA, 22 bp, Artificial Sequence
  synthetic primer
cagggaggaaacacaacagaaa

SEQ ID NO: 82 - DNA, 22 bp, Artificial Sequence
  synthetic primer
ttaggaggtcatagacgttgct

SEQ ID NO: 83 - DNA, 23 bp, Artificial Sequence
  synthetic primer
gctaactagcaatggtggtacag

SEQ ID NO: 84 - DNA, 23 bp, Artificial Sequence
  synthetic primer
gacgttcagtaaagtccagttga

SEQ ID NO: 85 - DNA, 21 bp, Artificial Sequence
  synthetic primer
cgtgagaaggagggtctcttg

SEQ ID NO: 86 - DNA, 21 bp, Artificial Sequence
  synthetic primer
gtgggaggtagtcagagtgtc

SEQ ID NO: 87 - DNA, 19 bp, Artificial Sequence
  synthetic primer
acgaaggtctgcgcgtgtt

SEQ ID NO: 88 - DNA, 20 bp, Artificial Sequence
  synthetic primer
ccgctggccatgaactacct

SEQ ID NO: 89 - DNA, 20 bp, Artificial Sequence
  synthetic primer
tgcagctgtgggttgattcc

SEQ ID NO: 90 - DNA, 23 bp, Artificial Sequence
  synthetic primer
aaacacgcacctcaaagctgttc

SEQ ID NO: 91 - DNA, 20 bp, Artificial Sequence
  synthetic primer
tccgcatcaggaaggctaga

SEQ ID NO: 92 - DNA, 19 bp, Artificial Sequence
  synthetic primer
aggaccaggcctccaagct

SEQ ID NO: 93 - DNA, 20 bp, Artificial
  synthetic primer
cccagagtttgagccgagtg

SEQ ID NO: 94 - DNA, 19 bp, Artificial Sequence
  synthetic primer
cccatcccttcgtcgtcct

SEQ ID NO: 95 - DNA, 20 bp, Artificial Sequence
  synthetic primer
gaacaggaggctgaaggggt

SEQ ID NO: 96 - DNA, 20 bp, Artificial Sequence
  synthetic primer
tcaggccatgctggtagacg

SEQ ID NO: 97 - DNA, 21 bp, Artificial Sequence
  synthetic siRNA
aagtattcattcctgcagaac

SEQ ID NO: 98 - DNA, 21 bp, Artificial Sequence
  synthetic siRNA
aacagcctggtgtacggcgcc